(19) United States
(12) Patent Application Publication
(10) Pub. No.: US 2019/0033306 A1
(43) Pub. Date: Jan. 31, 2019

(54) RECURRENT FUSION GENES IN HUMAN CANCERS

(71) Applicant: THE UNIVERSITY OF CHICAGO, Chicago, IL (US)

(72) Inventors: Kevin P. White, Chicago, IL (US); Chaitanya Bandlamudi, Chicago, IL (US)

(21) Appl. No.: 15/310,753

(22) PCT Filed: May 13, 2015
(86) PCT No.: PCT/US15/30677

§ 371 Date: Nov. 12, 2016

Related U.S. Application Data
(60) Provisional application No. 61/992,791, filed on May 13, 2014.

(51) Int. Cl. G01N 33/543; C07K 14/47; C07K 16/30

(52) U.S. Cl. CPC G01N 33/54353; C07K 14/47; C07K 16/30; C07K 2319/00; G01N 2800/56; G01N 2800/52; G01N 2800/7028

(57) ABSTRACT

Fusion transcripts are provided herein. In exemplary embodiments, the fusion transcript is encoded by a nucleic acid molecule comprising a general structure A-B, wherein structure A is a portion of a gene listed in Column A of Table 1 and structure B is a portion of a gene listed in Column B of Table 1, wherein the gene listed in Column A and the gene listed in Column B are listed in the same row of Table 1, wherein structure B is located immediately 3′ to structure A. Polypeptides encoded by the fusion transcript, nucleic acid molecules encoding the fusion transcript, and nucleic acid molecules comprising the reverse complement sequence of the fusion transcript, are additionally provided. Related expression vectors, host cells, binding agents, kits, and methods of using the same are further provided herein.

### CROSS-REFERENCE TO RELATED APPLICATIONS

This application claims the priority benefit of Provisional U.S. Patent Application No. 61/992,791, filed on May 13, 2014, which is incorporated by reference in its entirety.

### INCORPORATION BY REFERENCE OF MATERIAL SUBMITTED ELECTRONICALLY

Incorporated by reference in its entirety is a computer-readable nucleotide/amino acid sequence listing submitted concurrently herewith and identified as follows: 5,766,272 ASCII (Text) file named “48684A_SeqListing.txt,” created on May 13, 2015.

### BACKGROUND

Fusion genes are generated by genomic rearrangements that fuse domains from two distinct genes. Many fusions have been identified as driver mutations [Rowley et al., Nature 243(5405): 290-293 (1973); Soda et al., Nature 448(7153): 561-566 (2007)] and serve as effective therapeutic targets [Druker et al., N Engl J Med 344(14): 1031-1037 (2001); Kwak et al., N Engl J Med 363(18): 1693-1703 (2010)] in various cancers. Apart from a few highly recurrent fusion genes [Rowley et al., 1973, supra, Tomlins et al., Science 310(5748): 644-648 (2005)], a vast majority occur at low frequency [Perner et al., Neoplasia 10(3): 298-302 (2008), Wu et al., Cancer Discov 3(6): 636-647 (2013)], thereby rendering it difficult to identify and further analyze as a potential target for cancer therapy. While large sample sizes and fusion discovery methods aid in the process of low frequency fusion discovery, many methods suffer from a lack of sufficient sensitivity and/or specificity, and often times lead to the identification of false positives. Thus, highly sensitive methods of identifying fusions that occur at low frequency in cancer, and the identification of the fusions, are needed for advancing cancer diagnostics and therapy.

### SUMMARY

Provided herein are isolated fusion transcripts. Without being bound to any particular theory, the fusion transcripts provided herein are recurrent across multiple cancers and thus are useful in detecting cancer or a tumor in a subject. The fusion transcripts in some aspects encode a fusion polypeptide or a truncated polypeptide. The polypeptides encoded by the fusion transcripts also are believed to be useful in detecting and/or diagnosing cancer or a tumor in a subject and may serve as targets for anti-cancer or anti-tumor therapeutic agents.

In exemplary embodiments, the fusion transcript of the invention is encoded by a nucleic acid molecule comprising a general structure A-B, wherein structure A is a portion of a gene listed in Column A of Table 1 and structure B is a portion of a gene listed in Column B of Table 1, wherein the gene listed in Column A and the gene listed in Column B are listed in the same row of Table 1, wherein structure B is located immediately 3′ to structure A.

In exemplary aspects, the fusion transcript of the invention is encoded by a nucleic acid molecule comprising a structure A-B, wherein structure A is a portion of a gene listed in Column A of Table 1 and structure B is a portion of a gene listed in Column B of Table 1, wherein the gene listed in Column A and the gene listed in Column B are listed in the same row of Table 1 and the row is marked with an asterisk in the 2^{nd} column from the left, wherein structure B is located immediately 3′ to structure A.

In exemplary aspects, the fusion transcript of the invention is encoded by a nucleic acid molecule comprising a structure A-B, wherein structure A is a portion of a gene listed in Column A of Table 1 and structure B is a portion of a gene listed in Column B of Table 1, wherein the gene listed in Column A and the gene listed in Column B are listed in the same row of Table 1 and the row is not marked with a “#” in the 3^{rd} column from the left of Table 1, wherein structure B is located immediately 3′ to structure A.

In exemplary aspects, the fusion transcript of the invention is encoded by a nucleic acid molecule comprising a structure A-B, wherein structure A is a portion of a gene listed in Column A of Table 1 and structure B is a portion of a gene listed in Column B of Table 1, wherein the gene listed in Column A and the gene listed in Column B are listed in the same row of Table 1 and the the row is not marked with a “̂” in the 4^{th} column from the left, wherein structure B is located immediately 3′ to structure A.

Further embodiments and aspects of the fusion transcripts of the invention are provided herein.

Additionally provided herein are isolated polypeptides encoded by a fusion transcript of the invention. In exemplary aspects, the isolated polypeptide is a fusion polypeptide. In alternative aspects, the isolated polypeptide is a truncated polypeptide.

Isolated nucleic acid molecules are also provided herein. In exemplary embodiments, the isolated nucleic acid molecules encode a fusion transcript of the invention. In exemplary aspects, the isolated nucleic acid molecules comprise the reverse complement sequence of a fusion transcript. In exemplary aspects, the isolated nucleic acid molecules comprise sequence corresponding to an untranslated region of a gene.

Expression vectors are further provided herein. In exemplary embodiments, the expression vector comprises a fusion transcript of the invention. In exemplary embodiments, the expression vector comprises a nucleic acid molecule encoding a fusion transcript of the invention. In exemplary aspects, the expression vector comprises a nucleic acid molecule comprising the reverse complement sequence of a fusion transcript described herein. Provided herein are host cells comprising the expression vectors.

Also provided herein are binding agents. In exemplary embodiments, the binding agent specifically binds to a polypeptide encoded by a fusion transcript described herein. In exemplary embodiments, the binding agent specifically binds to a fusion transcript of the invention or to a nucleic acid molecule comprising the reverse complement sequence of a fusion transcript. In exemplary aspects, the binding agents specifically bind to a junction region of the fusion transcript, or of the polypeptide encoded thereby.

Kits comprising a binding agent of the invention is provided. In exemplary embodiments, the kit comprises a binding agent that specifically binds to a fusion polypeptide encoded by a fusion transcript encoded by a nucleic acid molecule comprising a structure A-B, wherein structure A is a portion of a gene listed in Column A of Table 1 and structure B is a portion of a gene listed in Column B of Table 1, wherein the gene listed in Column A and the gene listed in Column B are listed in the same row of Table 1, wherein structure B is located immediately 3′ to structure A. In exemplary aspects, the kit comprises a plurality of different binding agents, wherein each binding agent specifically binds to a different fusion polypeptide listed in one of Tables 1 to 4. In exemplary aspects, the kit comprises at least one binding agent that specifically binds to a fusion transcript encoded by a nucleic acid molecule comprising a structure A-B, wherein structure A is a portion of a gene listed in Column A of Table 1 and structure B is a portion of a gene listed in Column B of Table 1, wherein the gene listed in Column A and the gene listed in Column B are listed in the same row of Table 1 and the row is marked with an asterisk in the 2^{nd} column from the left, wherein structure B is located immediately 3′ to structure A. In exemplary aspects, the row is not marked with a “#” in the 3^{rd} column from the left of Table 1. In exemplary aspects, the row is not marked with a “̂” in the 4^{th} column from the left of Table 1. In exemplary aspects, the plurality collectively binds to each and every one of the fusion polypeptides listed in one of Tables 1 to 4.

Methods of detecting and/or diagnosing a cancer or a tumor in a subject are provided herein. In exemplary embodiments, the method comprises (i) contacting a binding agent that specifically binds to a polypeptide encoded by a fusion transcript of the invention with a sample obtained from the subject and (ii) determining the presence or absence of an immunoconjugate comprising the binding agent and the polypeptide, wherein a cancer or tumor is detected in the subject, when the immunoconjugate is determined as present. In exemplary embodiments, the method comprises (i) contacting one or more binding agents that specifically binds to a fusion transcript of the invention with a sample obtained from the subject, and (ii) determining (a) the structure of the molecule bound to the binding agent or (b) the presence or absence of a double stranded nucleic acid molecule comprising the binding agent and the fusion transcript, when the binding agent(s) bind(s) to either (a) a junction region of the fusion transcript comprising a portion of the 3′ end of structure A and a portion of the 5′ end of structure B, or (b) a portion of the structure A and portion of Structure B, wherein a cancer or tumor is detected in the subject, when the structure of the molecule is the structure of the fusion transcript or when the double stranded nucleic acid molecule is determined as present. In exemplary embodiments, the method comprises (i) generating a population of cDNAs from total RNA isolated from with a sample obtained from the subject, (ii) contacting one or more binding agent(s) which specifically bind(s) to a nucleic acid molecule comprising the reverse complement sequence of a fusion transcript, with a sample obtained from the subject, and (ii) determining (a) the structure of the molecule bound to the binding agent or (b) the presence or absence of a double stranded nucleic acid molecule comprising the binding agent(s) and the nucleic acid, when the binding agent binds to a sequence which is the reverse complement of a junction region of the fusion transcript comprising a portion of the 3′ end of structure A and a portion of the 5′ end of structure B, wherein a cancer or tumor is detected in the subject, when the structure of the molecule is the structure of the nucleic acid or when the double stranded nucleic acid molecule is determined as present.

In exemplary embodiments, the method of detecting and/or diagnosing a cancer or a tumor in a subject comprises (i) assaying a sample obtained from the subject for expression of a fusion transcript of the invention, expression of a polypeptide encoded by a fusion transcript of the invention, or presence of a nucleic acid molecule encoding a fusion transcript of the invention, when the sample is determined as positive for expression of the fusion transcript or expression of the polypeptide or presence of the nucleic acid molecule.

Methods of treating a cancer or a tumor in a subject are also provided herein. In exemplary embodiments, the method comprises (i) assaying a sample obtained from the subject for expression of a fusion transcript of the invention, a polypeptide encoded by a fusion transcript of the invention, or a nucleic acid molecule encoding a fusion transcript of the invention, and (ii) administering to the subject an anti-cancer therapeutic agent in an amount effective for treating a cancer or tumor, when the sample is determined as positive for expression of the fusion transcript or expression of the polypeptide or presence of the nucleic acid molecule.

Methods of determining a subject's need for an anti-cancer therapeutic agent is provided herein. In exemplary embodiments, the method comprises assaying a sample obtained from the subject for expression of a fusion transcript of the invention, a polypeptide encoded by a fusion transcript of the invention, or a nucleic acid molecule encoding a fusion transcript of the invention, wherein the subject needs an anti-cancer therapeutic agent, when the sample is determined as positive for expression of the fusion transcript, fusion polypeptide or nucleic acid molecule.

### BRIEF DESCRIPTION OF THE DRAWINGS

FIG. 1 represents a graph of the fold-change in proliferation (relative to control) for seven fusion gene cell lines.

FIG. 2 represents a graph of tumor growth over time post implantation of fusion cell lines.

FIG. 3 is an illustration of fusion genes and fusion gene transcripts.

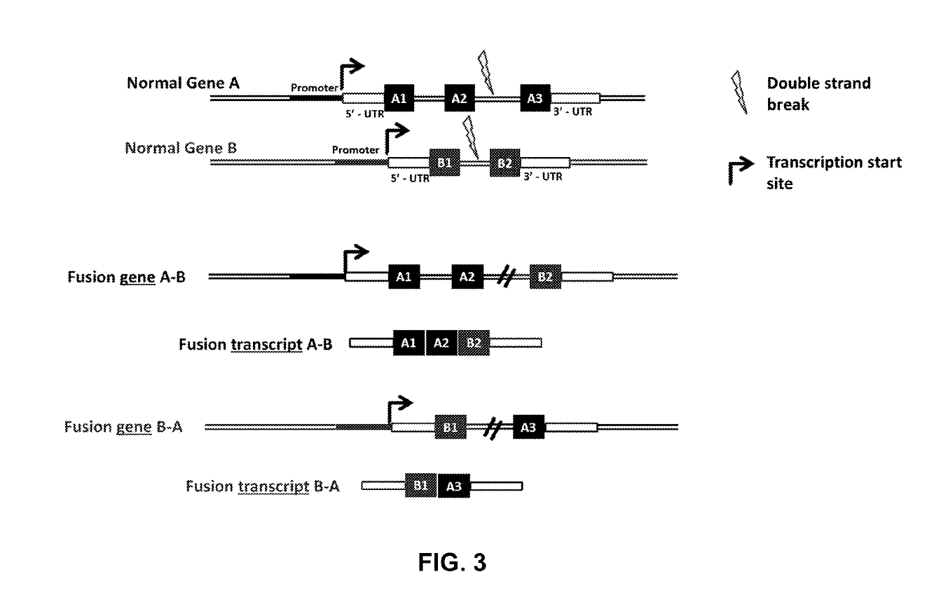

### DETAILED DESCRIPTION

The invention provides isolated nucleic acid molecules comprising a nucleotide sequence of novel fusion genes generated by genomic rearrangements that fuse domains from two distinct genes, and portions thereof, optionally, wherein the portion comprises the junction between the two genes. In exemplary aspects, the nucleic acid molecule comprises the nucleotide sequence (e.g., DNA sequence) of the full length fusion gene, including coding and non-coding sequence. In exemplary aspects, the nucleic acid molecule comprises the nucleotide sequence of only the coding sequence of the fusion gene. In exemplary aspects, the coding sequence encodes a transcript, e.g. an RNA transcript. In exemplary aspects, the transcript comprises fused domains encoded by two distinct genes and, in such aspects, the transcript is referenced herein as a “fusion transcript” or a “fusion gene transcript”. The invention provides isolated fusion transcripts as described herein. Further descriptions of the nucleic acid molecules and the fusion transcripts provided herein are provided below.

Fusion Transcripts

The invention provides novel fusion transcripts which are expressed in cancer cells or tumor cells. In exemplary aspects, the fusion transcript is encoded by a nucleic acid molecule comprising a general structure A-B, wherein structure A is a portion of a gene listed in Column A of Table 1 and structure B is a portion of a gene listed in Column B of Table 1, wherein the gene listed in Column A and the gene listed in Column B are listed in the same row of Table 1, wherein structure B is located immediately 3′ to structure A.

TABLE 1

| | | | | | | | | | | Reverse |
|---|---|---|---|---|---|---|---|---|---|---|
| | | | | | | Entrez | Entrez | Fusion CDS | | complement |
| | | | | | | Gene ID | Gene ID | cDNA | FL cDNA | of FL cDNA |
| Fusion Gene | * | # | \hat{ } | Column A | Column B | (Col. A) | (Col. B) | (SEQ ID NO:) | (SEQ ID NO:) | (SEQ ID NO:) |
| ACTN4_EIF3K | * | # | | ACTN4 | EIF3K | 81 | 27335 | 396-404 | 1396-1404 | 2396-2404 |
| ADAP1_GET4 | * | # | | ADAP1 | GET4 | 11033 | 51608 | 185-187 | 1185-1187 | 2185-2187 |
| ADRBK2_IGLL3P | * | # | | ADRBK2 | IGLL3P | 157 | 91353 | | | |
| AK125727_ANGEL1 | * | # | | AK125727 | ANGEL1 | | 23357 | | | |
| ARL15_NDUFS4 | * | | | ARL15 | NDUFS4 | 54622 | 4724 | 796-799 | 1796-1799 | 2796-2799 |
| ASCC1_MICU1 | * | | | ASCC1 | MICU1 | 51008 | 10367 | 299-310 | 1299-1310 | 2299-2310 |
| ASH1L_GON4L | * | | | ASH1L | GON4L | 55870 | 54856 | 42-60 | 1042-1060 | 2042-2060 |
| ATXN7_THOC7 | * | # | | ATXN7 | THOC7 | 6314 | 80145 | 108 | 1108 | 2108 |
| BC030525_LOC553103 | * | # | | BC030525 | LOC553103 | | 553103 | | | |
| BMPR1B_PDLIM5 | * | | | BMPR1B | PDLIM5 | 658 | 10611 | 453-475 | 1453-1475 | 2453-2475 |
| BRE_MRPL33 | * | # | | BRE | MRPL33 | 9577 | 9553 | 311-318 | 1311-1318 | 2311-2318 |
| C1orf63_TMEM50A | * | # | | C1orf63 | TMEM50A | 57035 | 23585 | | | |
| C7orf50_MAD1L1 | * | | | C7orf50 | MAD1L1 | 84310 | 8379 | 352-355 | 1352-1355 | 2352-2355 |
| CAPZA2_MET | * | | | CAPZA2 | MET | 830 | 4233 | 671-684 | 1671-1684 | 2671-2684 |
| CCAT1_LOC727677 | * | # | | CCAT1 | LOC727677 | | 727677 | | | |
| CCDC6_ANK3 | | | | CCDC6 | ANK3 | 8030 | 288 | 476-501 | 1476-1501 | 2476-2501 |
| CD44_PDHX | * | | | CD44 | PDHX | 960 | 8050 | 697-705 | 1697-1705 | 2697-2705 |
| CMTM7_CMTM8 | * | | | CMTM7 | CMTM8 | 112616 | 152189 | 348-351 | 1348-1351 | 2348-2351 |
| COL14A1_DEPTOR | * | | | COL14A1 | DEPTOR | 7373 | 64798 | 266-275 | 1266-1275 | 2266-2275 |
| CTSB_FDFT1 | * | # | | CTSB | FDFT1 | 1508 | 2222 | 576-590 | 1576-1590 | 2576-2590 |
| CUL4A_PCID2 | * | # | | CUL4A | PCID2 | 8451 | 55795 | 411-412 | 1411-1412 | 2411-2412 |
| DYNLRB1_ITCH | * | # | | DYNLRB1 | ITCH | 83658 | 83737 | 662 | 1662 | 2662 |
| EIF2C2_PTK2 | * | | | EIF2C2 | PTK2 | 27161 | 5747 | 502-509 | 1502-1509 | 2502-2509 |
| EIF3B_MAD1L1 | * | | | EIF3B | MAD1L1 | 8662 | 8379 | 116-132 | 1166-1132 | 2116-2132 |
| ESR1_CCDC170 | | | | ESR1 | CCDC170 | 2099 | 80129 | 720-725 | 1720-1725 | 2720-2725 |
| EXOC4_CHCHD3 | * | | | EXOC4 | CHCHD3 | 60412 | 54927 | 136-160 | 1136-1160 | 2136-2160 |
| EXT1_SAMD12 | * | | \hat{ } | EXT1 | SAMD12 | 2131 | 401474 | 800-801 | 1800-1801 | 2800-2801 |
| FAM162A_CCDC58 | * | # | | FAM162A | CCDC58 | 26355 | 131076 | | | |
| FAM190A_MMRN1 | * | | | FAM190A | MMRN1 | 401145 | 22915 | 685-687 | 1685-1687 | 2685-2687 |
| FAM3B_BACE2 | * | | | FAM3B | BACE2 | 54097 | 25825 | 340-347 | 1340-1347 | 2340-2347 |
| FANCL_VRK2 | * | # | | FANCL | VRK2 | 55120 | 7444 | 591-632 | 1591-1632 | 2591-2632 |
| FLJ22447_PRKCH | * | | \hat{ } | FLJ22447 | PRKCH | 400221 | 5583 | 133-134, | 1133-1134, | 2133-2134, |
| | | | | | | | | 802-803 | 1802-1803 | 2802-2803 |
| FRMD6_LOC283553 | * | | \hat{ } | FRMD6 | LOC283553 | 122786 | 283553 | 804-805 | 1804-1805 | 2804-2805 |
| FRS2_LYZ | * | | \hat{ } | FRS2 | LYZ | 10818 | 4069 | 806-807 | 1806-1807 | 2806-2807 |
| GTF2I_GTF2IRD1 | | | | GTF2I | GTF2IRD1 | 2969 | 9569 | 538-569 | 1538-1569 | 2538-2569 |
| HIAT1_SLC35A3 | * | # | | HIAT1 | SLC35A3 | 64645 | 23443 | 706-708 | 1706-1708 | 2706-2708 |
| HIF1A_PRKCH | * | # | | HIF1A | PRKCH | 3091 | 5583 | 170-179 | 1170-1179 | 2170-2179 |
| HP1BP3_EIF4G3 | * | | | HP1BP3 | EIF4G3 | 50809 | 8672 | 715-719 | 1715-1719 | 2715-2719 |
| IFT43_TTLL5 | * | | | IFT43 | TTLL5 | 112752 | 23093 | 291-293 | 1291-1293 | 2291-2293 |
| KAT6B_ADK | * | | | KAT6B | AD K | 23522 | 132 | 641-642 | 1641-1642 | 2641-2642 |
| KIF26B_SMYD3 | * | | | KIF26B | SMYD3 | 55083 | 64754 | 244-260 | 1244-1260 | 2244-2260 |
| LMO7_UCHL3 | * | | | LMO7 | UCH L3 | 4008 | 7347 | 663-670 | 1663-1670 | 2663-2670 |
| LOC100128675_LGI4 | * | # | | LOC100128675 | LGI4 | 100128675 | 163175 | 726-727 | 1726-1727 | 2726-2727 |
| LOC100133445_TNFRSF14 | * | # | | LOC100133445 | TNFRSF14 | 100133445 | 8764 | 661 | 1661 | 2661 |
| LOC100499467_SLC39A11 | * | | \hat{ } | LOC100499467 | SLC39A11 | 100499467 | 201266 | 808-809 | 1808-1809 | 2808-2809 |
| LRBA_SH3D19 | | | | LRBA | SH3D19 | 987 | 152503 | 534-537 | 1534-1537 | 2534-2537 |
| LYPD6_LYPD6B | * | | | LYPD6 | LYPD6B | 130574 | 130576 | 61-63 | 1061-1063 | 2061-2063 |
| MATR3_CTNNA1 | * | | | MATR3 | CTNNA1 | 9782 | 1495 | 103-106 | 1103-1106 | 2103-2106 |
| MBD3_UQCR11 | * | # | | MBD3 | UQCR11 | 53615 | 10975 | 107 | 1107 | 2107 |
| MLL5_LHFPL3 | * | | | MLL5 | LHFPL3 | 55904 | 375612 | 633-638 | 1633-1638 | 2633-2638 |
| MTAP_FLJ35282 | * | # | | MTAP | FLJ35282 | 4507 | 441389 | | | |
| MYH9_TXN2 | * | | | MYH9 | TXN2 | 4627 | 25828 | 521-524 | 1521-1524 | 2521-2524 |
| MYO6_SENP6 | | | | MYO6 | SENP6 | 4646 | 26054 | 394-395 | 1394-1395 | 2394-2395 |
| NCOA3_EYA2 | * | | | NCOA3 | EYA2 | 8202 | 2139 | 391-395 | 1391-1395 | 2391-2395 |
| NCOR2_SCARB1 | * | | | NCOR2 | SCARB1 | 9612 | 949 | 216-243 | 1216-1243 | 2216-2243 |
| NDRG1_B2M | * | # | | NDRG1 | B2M | 10397 | 567 | | | |
| NOC4L_FBRSL1 | * | # | | NOC4L | FBRSL1 | 79050 | 57666 | 709-710 | 1709-1710 | 2709-2710 |
| NSD1_ZNF346 | * | | | NSD1 | ZNF346 | 64324 | 23567 | 6-41 | | |
| NTN1_STX8 | * | # | | NTN1 | STX8 | 9423 | 9482 | 688-696 | 1688-1696 | 2688-2696 |
| PABPC1_YWHAZ | * | # | | PABPC1 | YWHAZ | 26986 | 7534 | 320-333 | 1320-1333 | 2320-2333 |
| PDE4D_DEPDC1B | * | | | PDE4D | DEPDC1B | 5144 | 55789 | 294-298 | 1294-1298 | 2294-2298 |
| PPFIBP1_C12orf70 | * | | \hat{ } | PPFIBP1 | C12orf70 | 8496 | 341346 | 810 | 1810 | 2810 |
| PPP1CB_PLB1 | * | | | PPP1CB | PLB1 | 5500 | 151056 | 188-202 | 1188-1202 | 2188-2202 |
| PTPRK_RSPO3 | | | | PTPRK | RSPO3 | 5796 | 84870 | 510-520 | 1510-1520 | 2510-2520 |
| QKI_PACRG | * | | | QKI | PACRG | 9444 | 135138 | 276-279 | 1276-1279 | 2276-2279 |
| RAB40C_TMEM8A | * | # | | RAB40C | TMEM8A | 57799 | 58986 | 204 | 1204 | 2204 |
| RB1_ITM2B | | | | RB1 | ITM2B | 5925 | 9445 | 659-660 | 1659-1660 | 2659-2660 |
| REV3L_FYN | * | # | | REV3L | FYN | 5980 | 2534 | 109-115 | 1109-1115 | 2109-2115 |
| RMST_C9orf3 | * | # | | RMST | C9orf3 | 196475 | 84909 | | | |
| RPL39L_ST6GAL1 | * | # | | RPL39L | ST6GAL1 | 116832 | 6480 | 639-640 | 1639-1640 | 2639-2640 |
| RPS15A_ARL6IP1 | * | # | | RPS15A | ARL6IP1 | 6210 | 23204 | 261-265 | 1261-1265 | 2261-2265 |
| RPS6KB1_VMP1 | | | | RPS6KB1 | VMP1 | 6197 | 81671 | 413-452 | 1413-1452 | 2413-2452 |
| SGK1_AJ606331 | * | # | | SGK1 | AJ606331 | 6446 | | | | |
| SH3PXD2A_OBFC1 | * | | | SH3PXD2A | OBFC1 | 9644 | 79991 | 100-102 | 1100-1102 | 2100-2102 |
| SKP1_CDKL3 | | | | SKP1 | CDKL3 | 6500 | 51625 | 406-410 | 1406-1410 | 2406-2410 |
| SLPI_WFDC2 | * | | | SLPI | WFDC2 | 6590 | 10406 | 532-533 | 1532-1533 | 2532-2533 |
| SMARCC1_MAP4 | * | | | SMARCC1 | MAP4 | 6599 | 4134 | 64-99 | 1064-1099 | 2064-2099 |
| SNX29P1_CRYM-AS1 | * | # | | SNX29P1 | CRYM-AS1 | 400509 | 400508 | | | |
| SOLH_TMEM8A | * | # | | SOLH | TMEM8A | 6650 | 58986 | 405 | 1405 | 2405 |
| SORL1_TECTA | * | | | SORL1 | TECTA | 6653 | 7007 | 1-5 | | |
| SRPK2_PUS7 | * | | | SRPK2 | PUS7 | 6733 | 54517 | 182-184 | 1182-1184 | 2182-2184 |
| ST6GAL1_RPL39L | * | # | | ST6GAL1 | RPL39L | 6480 | 116832 | 135 | 1135 | 2135 |
| STX5_WDR74 | * | | | STX5 | WDR74 | 6811 | 54663 | 525-531 | 1525-1531 | 2525-2531 |
| TANC1_PKP4 | * | | | TANC1 | PKP4 | 85461 | 8502 | 356-367 | 1356-1367 | 2356-2367 |
| TFDP1_TMCO3 | * | | | TFDP1 | TMCO3 | 7027 | 55002 | 280-290 | 1280-1290 | 2280-2290 |
| THSD4_LRRC49 | * | | | THSD4 | LRRC49 | 79875 | 54839 | 207-215 | 1207-1215 | 2207-2215 |
| TLK2_METTL2B | * | | | TLK2 | METTL2B | 11011 | 55798 | | | |
| TNRC18_RNF216 | * | | \hat{ } | TNRC18 | RNF216 | 84629 | 54476 | 575, 811 | 1575, 1811 | 2575, 2811 |
| TRPS1_EIF3H | * | # | | TRPS1 | EIF3H | 7227 | 8667 | 368-385 | 1368-1385 | 2368-2385 |
| TTC6_MIPOL1 | * | | | TTC6 | MIPOL1 | 319089 | 145282 | | | |
| TTYH3_MAD1L1 | * | | | TTYH3 | MAD1L1 | 80727 | 8379 | 643-658 | 1643-1658 | 2643-2658 |
| UBE2E1_UBE2E2 | * | # | | UBE2E1 | UBE2E2 | 7324 | 7325 | 711-714 | 1711-1714 | 2711-2714 |
| UBE2Z_SNF8 | * | # | | UBE2Z | SNF8 | 65264 | 11267 | 334-339 | 1334-1339 | 2334-2339 |
| USP22_MYH10 | * | | | USP22 | MYH10 | 23326 | 4628 | 161-169 | 1161-1169 | 2161-2169 |
| VAPB_GNAS | * | # | | VAPB | GNAS | 9217 | 2778 | 386-390 | 1386-1390 | 2386-2390 |
| VRK2_FANCL | * | # | | VRK2 | FANCL | 7444 | 55120 | 728-795 | 1728-1795 | 2728-2795 |
| WASF2_AHDC1 | * | | | WASF2 | AHDC1 | 10163 | 27245 | 205-206 | 1205-1206 | 2205-2206 |
| XKR9_LACTB2 | * | # | | XKR9 | LACTB2 | 389668 | 51110 | | | |
| XPR1_BC036830 | * | # | | XPR1 | BC036830 | 9213 | | | | |
| YWHAE_CRK | * | # | | YWHAE | CRK | 7531 | 1398 | 180-181 | 1180-1181 | 2180-2181 |
| YWHAE_GNAS | * | # | | YWHAE | GNAS | 7531 | 2778 | 570-574 | 1570-1574 | 2570-2574 |
| ZBTB20_LSAMP | * | | \hat{ } | ZBTB20 | LSAMP | 26137 | 4045 | 812 | 1812 | 2812 |
| ZC3H7A_BCAR4 | * | | | ZC3H7A | BCAR4 | 29066 | 400500 | 319 | 1319 | 2319 |
| ZFYVE21_KLC1 | * | # | | ZFYVE21 | KLC1 | 79038 | 3831 | 203 | 1203 | 2203 |
| DNAJC24_IMMP1L | * | | | DNAJC24 | IMMP1L | 120526 | 196294 | 813 | 1813 | 2813 |
| GRB7_ERBB2 | * | | | GRB7 | ERBB2 | 2886 | 2064 | 814-824 | 1814-1824 | 2814-2824 |
| LITAF_BCAR4 | * | | | LITAF | BCAR4 | 9516 | 400500 | 825-828 | 1825-1828 | 2825-2828 |
| REXO1_KLF16 | * | | | REXO1 | KLF16 | 57455 | 83855 | 836 | 1836 | 2836 |
| RGNEF_BTF3 | * | | | RGNEF | BTF3 | 64283 | 689 | 837-840 | 1837-1840 | 2837-2840 |
| TYMS_SEPT9 | * | | | TYMS | SEPT9 | 7298 | 10801 | 843 | 1843 | 2843 |
| WASF2_IFI6 | * | | | WASF2 | IF16 | 10163 | 2537 | 844 | | |

“*” Novel fusion transcript
“#” fusions that were detected at <5× enrichment in primary tumors, relative to the 3,600 cell line and tissue transcriptomes from healthy individuals.
“\hat{ }” out of frame
CDS = coding sequence
FL = full length

In exemplary aspects, the fusion transcript is encoded by a nucleic acid molecule comprising a general structure A-B, wherein structure A is a portion of a gene listed in Column A of Table 1 and structure B is a portion of a gene listed in Column B of Table 1, wherein the gene listed in Column A and the gene listed in Column B are listed in the same row of Table 1 and the row is marked with an asterisk in the 2^{nd} column from the left, wherein structure B is located immediately 3′ to structure A. These fusion transcripts are believed to be novel.

In exemplary aspects, the fusion transcript is encoded by a nucleic acid molecule comprising a general structure A-B, wherein structure A is a portion of a gene listed in Column A of Table 1 and structure B is a portion of a gene listed in Column B of Table 1, wherein the gene listed in Column A and the gene listed in Column B are listed in the same row of Table 1 and the row is not marked with a “#” in the 3^{rd} column from the left, wherein structure B is located immediately 3′ to structure A. These fusion transcripts not having a “#” in the 3rd column are believed to be present in primary tumors at a level which is at least 5× that found in healthy individuals.

In exemplary aspects, the fusion transcript is encoded by a nucleic acid molecule comprising a general structure A-B, wherein structure A is a portion of a gene listed in Column A of Table 1 and structure B is a portion of a gene listed in Column B of Table 1 and the row is not marked with a “̂” in the 4^{th} column from the left, wherein the gene listed in Column A and the gene listed in Column B are listed in the same row of Table 1, wherein structure B is located immediately 3′ to structure A. These fusion transcripts not having a “̂” in the 4^{th} column are believed to be in frame.

In exemplary aspects, the fusion transcript of the invention is encoded by a nucleic acid molecule comprising a structure A-B, wherein structure A is a portion of a gene listed in Column A of Table 1 and structure B is a portion of a gene listed in Column B of Table 1, wherein the gene listed in Column A and the gene listed in Column B are listed in the same row of Table 1 and the row is (a) marked with an asterisk in the 2^{nd} column from the left, (b) not marked with a “#” in the 3^{rd} column from the left, (c) not marked with a “̂” in the 4^{th} column from the left, or (d) a combination thereof, wherein structure B is located immediately 3′ to structure A. In exemplary aspects, the row is marked with an asterisk in the 2^{nd} column from the left, not marked with a “#” in the 3^{rd} column from the left, and not marked with a “̂” in the 4^{th} column from the left. In exemplary aspects, the row is marked with an asterisk in the 2^{nd} column from the left, not marked with a “#” in the 3^{rd} column from the left, but is marked with a “̂” in the 4^{th} column from the left. In exemplary aspects, the row is marked with an asterisk in the 2^{nd} column from the left, marked with a “#” in the 3^{rd} column from the left, and is not marked with a “̂” in the 4^{th} column from the left. In exemplary aspects, the row is not marked with an asterisk in the 2^{nd} column from the left, not marked with a “#” in the 3^{rd} column from the left, and not marked with a “̂” in the 4^{th} column from the left.

In exemplary aspects, the fusion transcript is encoded by a nucleic acid molecule comprising a general structure A-B, wherein structure A is a portion of a gene listed in Column A of Table 2 and structure B is a portion of a gene listed in Column B of Table 2, wherein the gene listed in Column A and the gene listed in Column B are listed in the same row of Table 2, wherein structure B is located immediately 3′ to structure A. Table 2 lists a subset of the fusion transcripts listed in Table 1 which have been validated or are in the process of being validated.

TABLE 2

| | | | | | Fusion | |
|---|---|---|---|---|---|---|
| | | | Entrez | Entrez | Polypeptide | Col. A Gene Name/Entrez Gene |
| | | | Gene ID | Gene ID | (SEQ ID | ID/Col. B Gene Name/Entrez |
| Fusion Gene | Column A | Column B | (Col. A) | (Col. B) | NOs:) | Gene ID |
| ARL15_NDUFS4 | ARL15 | NDUFS4 | 54622 | 4724 | 796-799 | ARL15\|54622_NDUFS4\|4724 |
| BMPR1B_PDLIM5 | BMPR1B | PDLIM5 | 658 | 10611 | 453-475 | BMPR1B\|658_PDLIM5\|10611 |
| CAPZA2_MET | CAPZA2 | MET | 830 | 4233 | 671-684 | CAPZA2\|830_MET\|4233 |
| CD44_PDHX | CD44 | PDHX | 960 | 8050 | 697-705 | CD44\|960_PDHX\|8050 |
| LMO7_UCHL3 | LMO7 | UCHL3 | 4008 | 7347 | 663-670 | LMO7\|4008_UCHL3\|7347 |
| MATR3_CTNNA1 | MATR3 | CTNNA1 | 9782 | 1495 | 103-106 | MATR3\|9782_CTNNA1\|1495 |
| PPP1CB_PLB1 | PPP1CB | PLB1 | 5500 | 151056 | 188-202 | PPP1CB\|5500_PLB1\|151056 |
| SORL1_TECTA | SORL1 | TECTA | 6653 | 7007 | 1-5 | SORL1\|6653_TECTA\|7007 |
| TTYH3_MAD1L1 | TTYH3 | MAD1L1 | 80727 | 8379 | 643-658 | TTYH3\|80727_MAD1L1\|8379 |
| USP22_MYH10 | USP22 | MYH10 | 23326 | 4628 | 161-169 | USP22\|23326_MYH10\|4628 |
| ZC3H7A_BCAR4 | ZC3H7A | BCAR4 | 29066 | 400500 | 319 | ZC3H7A\|29066_BCAR4\|400500 |

In exemplary aspects, the fusion transcript encoded by a nucleic acid molecule comprising a general structure A-B, wherein structure A is a portion of a gene listed in Column A of Table 3 and structure B is a portion of a gene listed in Column B of Table 3, wherein the gene listed in Column A and the gene listed in Column B are listed in the same row of Table 3, wherein structure B is located immediately 3′ to structure A. Table 3 lists a subset of fusion transcripts listed in Table 1 which have been subjected to in vitro growth assays.

TABLE 3

| | | | | | Fusion | |
|---|---|---|---|---|---|---|
| | | | Entrez | Entrez | Polypeptide | Col. A Gene Name/Entrez Gene |
| | | | Gene ID | Gene ID | (SEQ ID | ID/Col. B Gene Name/Entrez |
| Fusion Gene | Column A | Column B | (Col. A) | (Col. B) | NOs:) | Gene ID |
| ARL15_NDUFS4 | ARL15 | NDUFS4 | 54622 | 4724 | 796-799 | ARL15\|54622_NDUFS4\|4724 |
| BMPR1B_PDLIM5 | BMPR1B | PDLIM5 | 658 | 10611 | 453-475 | BMPR1B\|658_PDLIM5\|10611 |
| CAPZA2_M ET | CAPZA2 | MET | 830 | 4233 | 671-684 | CAPZA2\|830_MET\|4233 |
| CD44_PDHX | CD44 | PDHX | 960 | 8050 | 697-705 | CD44\|960_PDHX\|8050 |
| LMO7_UCHL3 | LMO7 | UCHL3 | 4008 | 7347 | 663-670 | LMO7\|4008_UCHL3\|7347 |
| ZC3H7A_BCAR4 | ZC3H7A | BCAR4 | 29066 | 400500 | 319 | ZC3H7A\|29066_BCAR4\|400500 |

In exemplary aspects, the fusion transcript encoded by a nucleic acid molecule comprising a general structure A-B, wherein structure A is a portion of a gene listed in Column A of Table 4 and structure B is a portion of a gene listed in Column B of Table 4, wherein the gene listed in Column A and the gene listed in Column B are listed in the same row of Table 4, wherein structure B is located immediately 3′ to structure A. Table 4 lists a subset of fusion transcripts listed in Table 1 which have been subjected to tumor growth assays.

TABLE 4

| | | | | | Fusion | |
|---|---|---|---|---|---|---|
| | | | Entrez | Entrez | Polypeptide | Col. A Gene Name/Entrez Gene |
| | | | Gene ID | Gene ID | (SEQ ID | ID/Col. B Gene Name/Entrez |
| Fusion Gene | Column A | Column B | (Col. A) | (Col. B) | NOs:) | Gene ID |
| BMPR1B_PDLIM5 | BMPR1B | PDLIM5 | 658 | 10611 | 453-475 | BMPR1B\|658_PDLIM5\|10611 |
| LMO7_UCHL3 | LMO7 | UCHL3 | 4008 | 7347 | 663-670 | LMO7\|4008_UCHL3\|7347 |
| ZC3H7A_BCAR4 | ZC3H7A | BCAR4 | 29066 | 400500 | 319 | ZC3H7A\|29066_BCAR4\|400500 |

In accordance with the above descriptions, the fusion transcript provided herein is encoded by a nucleic acid molecule comprising a general structure A-B, wherein each of structure A and structure B is a portion of a gene and wherein structure A is a portion of a gene which is different from the gene of structure B. In exemplary aspects, structure A is a portion of at least 50 nucleotides of the gene listed in Column A and structure B is a portion of at least 50 nucleotides of the gene listed in Column B. In exemplary aspects, structure A is a portion of at least 60 nucleotides of the gene listed in Column A and structure B is a portion of at least 100 nucleotides of the gene listed in Column B. In exemplary aspects, structure A is a portion of at least 65 nucleotides of the gene listed in Column A and structure B is a portion of at least 200 nucleotides of the gene listed in Column B. In exemplary aspects, structure A is a portion of at least 65 nucleotides of the gene listed in Column A and structure B is a portion of at least 250 nucleotides of the gene listed in Column B. In exemplary aspects, structure A is a portion of at least 65 nucleotides of the gene listed in Column A and structure B is a portion of at least 275 nucleotides of the gene listed in Column B.

In accordance with the above descriptions, the fusion transcript provided herein is encoded by a nucleic acid molecule comprising a general structure A-B, wherein each of structure A and structure B is a portion of a gene, wherein structure A is a portion of a gene which is different from the gene of structure B, and the point at which structure A ends and structure B begins is recognized as a junction.

In exemplary aspects, the fusion transcript is encoded by a nucleic acid molecule comprising a general structure A-B, wherein each of structure A and structure B is a portion of a gene comprising exons. In exemplary aspects, the exons of the gene of structure A is in frame with the exons of the gene of structure B. In exemplary aspects, the fusion transcript encodes a fusion polypeptide comprising a portion encoded by the gene listed in Column A and a portion encoded by the gene listed in Column B. In exemplary aspects, the exons of the gene of structure A is out of frame with the exons of the gene of structure B. In such aspects, the fusion transcript may not encode a fusion polypeptide comprising a portion encoded by the gene listed in Column A and a portion encoded by the gene listed in Column B. Rather, the fusion transcript may encode a fusion polypeptide comprising a portion encoded by the gene listed in Column A and not in Column B, or the fusion transcript may not encode a polypeptide.

In alternative exemplary aspects, the fusion transcript is encoded by a nucleic acid molecule comprising a general structure A-B, wherein only one of structure A and structure B is a portion of a gene comprising exons. In exemplary aspects, the fusion transcript encodes a polypeptide comprising at least a portion encoded by only one of the genes listed in Column A and the genes listed in Column B.

In yet other exemplary aspects, the fusion transcript is encoded by a nucleic acid molecule comprising a general structure A-B, wherein neither structure A nor structure B is a portion of a gene comprising exons. In exemplary aspects, the fusion transcript does not encode a polypeptide.

In exemplary aspects, the fusion transcripts described herein are isolated. As used herein, the term “isolated” refers to a product having been removed from its natural environment. In the instant case, the fusion transcripts of the invention are removed from intracellular components of a cancer or tumor cell. In exemplary aspects, the fusion transcript of the invention exists in a composition and the composition has a given % purity with regard to the fusion transcript. For example, the purity of the compositions may be in exemplary aspects at least about 50%, can be greater than 60%, 70% or 80%, or can be 100%.

In exemplary aspects, the fusion transcripts described herein comprise ribonucleotides. In exemplary aspects, the ribonucleotides comprise a nucleobase, selected from the group consisting of uracil, adenine, guanine, cytosine. In exemplary aspects, the ribonucleotides are linked via phosphodiester bonds. Also, in exemplary aspects, the fusion transcripts of the invention are single stranded. In exemplary aspects, the fusion transcripts provided herein are not cyclic, although the fusion transcripts may comprise secondary or tertiary structural features, including, e.g., stem loop structures, and the like.

The sequence listing provides nucleotide sequences of complementary DNA (cDNA) of fusion transcripts of the invention. The nucleotide sequences of SEQ ID NOs: 1-844 represent the coding sequence portion of the cDNA of the fusion transcripts of the invention, while the nucleotide sequences of SEQ ID NOs: 1001-1844 represent the full length cDNA of the fusion transcripts of the invention. The latter group of sequences in some aspects contain both coding and non-coding sequences.

In exemplary embodiments of the invention, the fusion transcript comprises a nucleotide sequence which is the reverse complement of any one of SEQ ID NOs: 1 to 799. The reverse complement in some aspects is the reverse complement RNA sequence. For a sequence AGTC, which by convention is understood to be written in the 5′→3′ direction, the complement sequence is TCAG, the reverse complement sequence is GACT, and the reverse complement RNA sequence is GACU. In exemplary embodiments, the fusion transcript comprises a nucleotide sequence which is the reverse complement (e.g., the reverse complement RNA) of any one of SEQ ID NOs: 800 to 844. In exemplary embodiments, the fusion transcript comprises a nucleotide sequence which is the reverse complement (e.g., the reverse complement RNA) of any one of SEQ ID NOs: 1-844. In exemplary aspects, the fusion transcript comprises a nucleotide sequence which is the reverse complement (e.g., the reverse complement RNA) of any one of the SEQ ID NOs: listed in the 9^{th} column from the left of Table 1. In exemplary aspects, the fusion transcript comprises a nucleotide sequence which is the reverse complement (e.g., the reverse complement RNA) of any one of the SEQ ID NOs: listed in the 9^{th} column from the left of Table 1 in a row having a “*” in the 2^{nd} column to the left of Table 1. In exemplary aspects, the fusion transcript comprises a nucleotide sequence which is the reverse complement (e.g., the reverse complement RNA) of any one of the SEQ ID NOs: listed in the 9^{th} column from the left of Table 1 in a row not marked with a “#” in the 3rd column to the left of Table 1. In exemplary aspects, the fusion transcript comprises a nucleotide sequence which is the reverse complement (e.g., the reverse complement RNA) of any one of the SEQ ID NOs: listed in the 9^{th} column from the left of Table 1 in a row not marked with a “̂” in the 4th column to the left of Table 1. In exemplary aspects, the fusion transcript comprises a nucleotide sequence which is the reverse complement (e.g., the reverse complement RNA) of any one of the SEQ ID NOs: listed in the 9^{th} column from the left of Table 1 in a row (a) with a “*” in the 2^{nd} column to the left of Table 1, (b) not marked with a “#” in the 3rd column to the left of Table 1, (c) not marked with a “̂” in the 4th column to the left of Table 1, or (d) a combination thereof.

In exemplary embodiments, the fusion transcript comprises a nucleotide sequence which is the reverse complement (e.g., the reverse complement RNA) of any one of SEQ ID NOs: 1001 to 1799. In exemplary embodiments, the fusion transcript comprises a nucleotide sequence which is the reverse complement (e.g., the reverse complement RNA) of any one of SEQ ID NOs: 1800 to 1844. In exemplary embodiments, the fusion transcript comprises a nucleotide sequence which is the reverse complement (e.g., the reverse complement RNA) of any one of SEQ ID NOs: 1001-1844. In exemplary aspects, the fusion transcript comprises a nucleotide sequence which is the reverse complement (e.g., the reverse complement RNA) of any one of the SEQ ID NOs: listed in the 2nd column from the right of Table 1. In exemplary aspects, the fusion transcript comprises a nucleotide sequence which is the reverse complement (e.g., the reverse complement RNA) of any one of the SEQ ID NOs: listed in the 2nd column from the right of Table 1 in a row having a “*” in the 2^{nd} column to the left of Table 1. In exemplary aspects, the fusion transcript comprises a nucleotide sequence which is the reverse complement (e.g., the reverse complement RNA) of any one of the SEQ ID NOs: listed in the 2nd column from the right of Table 1 in a row not marked with a “#” in the 3rd column to the left of Table 1. In exemplary aspects, the fusion transcript comprises a nucleotide sequence which is the reverse complement (e.g., the reverse complement RNA) of any one of the SEQ ID NOs: listed in the 2nd column from the right of Table 1 in a row not marked with a “̂” in the 4th column to the left of Table 1. In exemplary aspects, the fusion transcript comprises a nucleotide sequence which is the reverse complement (e.g., the reverse complement RNA) of any one of the SEQ ID NOs: listed in the 2nd column from the right of Table 1 in a row (a) marked with a “*” in the 2^{nd} column to the left of Table 1, (b) not marked with a “#” in the 3rd column to the left of Table 1, (c) not marked with a “̂” in the 4th column to the left of Table 1, or (d) a combination thereof.

In exemplary embodiments, the fusion transcript comprises a nucleotide sequence of any one of SEQ ID NOs: 2001 to 2844. In exemplary aspects, the fusion transcript comprises a nucleotide sequence of any one of the SEQ ID NOs: listed in the right most column of Table 1. In exemplary aspects, the fusion transcript comprises a nucleotide sequence of any one of the SEQ ID NOs: listed in the right most column of Table 1 in a row having a “*” in the 2^{nd} column to the left of Table 1. In exemplary aspects, the fusion transcript comprises a nucleotide sequence of any one of the SEQ ID NOs: listed in the right most column of Table 1 in a row not marked with a “#” in the 3rd column to the left of Table 1. In exemplary aspects, the fusion transcript comprises a nucleotide sequence of any one of the SEQ ID NOs: listed in the right most column of Table 1 in a row not marked with a “̂” in the 4th column to the left of Table 1. In exemplary aspects, the the fusion transcript comprises a nucleotide sequence of any one of the SEQ ID NOs: listed in the right most column of Table 1 in a row (a) marked with a “*” in the 2^{nd} column to the left of Table 1, (b) not marked with a “#” in the 3rd column to the left of Table 1, (c) not marked with a “̂” in the 4th column to the left of Table 1, or (d) a combination thereof.

With regard to the fusion transcripts listed in Table 1, the location of the junction between structure A and structure B for each of SEQ ID NOs: 1-844, if present, and the location of the junction between structure A and structure B for each of SEQ ID NOs: 1001-1844, if present, is described in Table 5, found after the EXAMPLES section. In exemplary aspects, some of the sequences of SEQ ID NOs: 1-844 do not have a junction and therefore do not encode a fusion polypeptide.

Polypeptides Encoded by Fusion Transcripts

The invention provides isolated polypeptides. In exemplary embodiments, the polypeptide of the invention is encoded by a fusion transcript described herein. In exemplary aspects, the polypeptide of the invention comprises a general structure A-B and is encoded by a nucleotide sequence comprising (i) at least a portion of the gene listed in Column A of Table 1 as structure A and (ii) at least a portion of the gene listed in Column B of Table 1 as structure B.

In exemplary embodiments, the polypeptide of the invention is encoded by a fusion transcript encoded by a nucleic acid molecule comprising a general structure A-B, wherein structure A is a portion of a gene listed in Column A of Table 1 and structure B is a portion of a gene listed in Column B of Table 1, wherein the gene listed in Column A and the gene listed in Column B are listed in the same row of Table 1, wherein structure B is located immediately 3′ to structure A.

In exemplary embodiments, the polypeptide of the invention is encoded by a fusion transcript encoded by a nucleic acid molecule comprising a general structure A-B, wherein structure A is a portion of a gene listed in Column A of Table 1 and structure B is a portion of a gene listed in Column B of Table 1, wherein the gene listed in Column A and the gene listed in Column B are listed in the same row of Table 1 and the row is marked with an asterisk in the 2^{nd} column from the left, wherein structure B is located immediately 3′ to structure A.

In exemplary embodiments, the polypeptide of the invention is encoded by a fusion transcript encoded by a nucleic acid molecule comprising a general structure A-B, wherein structure A is a portion of a gene listed in Column A of Table 1 and structure B is a portion of a gene listed in Column B of Table 1, wherein the gene listed in Column A and the gene listed in Column B are listed in the same row of Table 1 and the row is not marked with a “#” in the 3^{rd} column from the left, wherein structure B is located immediately 3′ to structure A.

In exemplary embodiments, the polypeptide is encoded by a fusion transcript encoded by a nucleic acid molecule comprising a general structure A-B, wherein structure A is a portion of a gene listed in Column A of Table 1 and structure B is a portion of a gene listed in Column B of Table 1, wherein the gene listed in Column A and the gene listed in Column B are listed in the same row of Table 1 and the row is (a) marked with an asterisk in the 2^{nd} column from the left, (b) not marked with a “#” in the 3^{rd} column from the left, (c) not marked with a “̂” in the 4^{th} column from the left, or (d) a combination thereof, wherein structure B is located immediately 3′ to structure A.

In exemplary embodiments, the polypeptide of the invention is encoded by a fusion transcript encoded by a nucleic acid molecule comprising a general structure A-B, wherein structure A is a portion of a gene listed in Column A of Table 2 and structure B is a portion of a gene listed in Column B of Table 2, wherein the gene listed in Column A and the gene listed in Column B are listed in the same row of Table 2, wherein structure B is located immediately 3′ to structure A.

In exemplary embodiments, the polypeptide of the invention is encoded by a fusion transcript encoded by a nucleic acid molecule comprising a general structure A-B, wherein structure A is a portion of a gene listed in Column A of Table 3 and structure B is a portion of a gene listed in Column B of Table 3, wherein the gene listed in Column A and the gene listed in Column B are listed in the same row of Table 3, wherein structure B is located immediately 3′ to structure A.

In exemplary embodiments, the polypeptide of the invention is encoded by a fusion transcript encoded by a nucleic acid molecule comprising a general structure A-B, wherein structure A is a portion of a gene listed in Column A of Table 4 and structure B is a portion of a gene listed in Column B of Table 4, wherein the gene listed in Column A and the gene listed in Column B are listed in the same row of Table 4, wherein structure B is located immediately 3′ to structure A.

In exemplary aspects, the polypeptide of the invention is encoded by a fusion transcript comprising a nucleotide sequence which is the reverse complement (e.g., the reverse complement RNA) of any one of SEQ ID NOs: 1 to 799. In exemplary aspects, the polypeptide of the invention is encoded by a fusion transcript comprising a nucleotide sequence which is the reverse complement (e.g., the reverse complement RNA) of any one of SEQ ID NOs: 800 to 844. In exemplary aspects, the polypeptide of the invention is encoded by a fusion transcript comprising a nucleotide sequence which is the reverse complement (e.g., the reverse complement RNA) of any one of SEQ ID NOs: 1001 to 1799. In exemplary aspects, the polypeptide of the invention is encoded by a fusion transcript comprising a nucleotide sequence which is the reverse complement (e.g., the reverse complement RNA) of any one of SEQ ID NOs: 1800 to 1844. In exemplary aspects, the polypeptide of the invention is encoded by a fusion transcript comprising a nucleotide sequence of any one of SEQ ID NOs: 2001 to 2844. In exemplary aspects, the fusion polypeptide is encoded by the reverse complement (e.g., the reverse complement RNA) of any one of SEQ ID NOs: 1-8, 10-35, 37-39, 41, 44, 45, 46, 48-51, 53-55, 58, 60, 64-102, 116, 117, 119, 121-124, 126-129, 130-132, 136, 137, 139, 140, 142-156, 158, 159, 161-169, 183, 184, 188-202, 207-240, 242, 243, 245-256, 258-260, 266-281, 283-297, 299-310, 340-355, 453, 454, 456-458, 461, 462, 464-466, 469, 471, 475, 502-504, 506-508, 521, 525, 527, 528, 530, 532-537, 575, 633-638, 641-658, 663-680, 682-684, 697-705, 718, 796-814, 816, 817, 819, 836-838, and 840-843. In exemplary aspects, the fusion polypeptide is encoded by the reverse complement (e.g., the reverse complement RNA) of any one of SEQ ID NOs: 1001-1008, 1010-1035, 1037-1039, 1041, 1044, 1045, 1046, 1048-1051, 1053-1055, 1058, 1060, 1064-1102, 1116, 1117, 1119, 1121-1124, 1126-1129, 1130-1132, 1136, 1137, 1139, 1140, 1142-1156, 1158, 1159, 1161-1169, 1183, 1184, 1188-1202, 1207-1240, 1242, 1243, 1245-1256, 1258-1260, 1266-1281, 1283-1297, 1299-1310, 1340-1355, 1453, 1454, 1456-1458, 1461, 1462, 1464-1466, 1469, 1471, 1475, 1502-1504, 1506-1508, 1521, 1525, 1527, 1528, 1530, 1532-1537, 1575, 1633-1638, 1641-1658, 1663-1680, 1682-1684, 1697-1705, 1718, 1796-1814, 1816, 1817, 1819, 1836-1838, 1840-1843. In exemplary aspects, the fusion polypeptide is encoded by the reverse complement (e.g., the reverse complement RNA) of any one of the SEQ ID NOs: listed in Table 5.

In exemplary aspects, the polypeptide of the invention is encoded by a fusion transcript comprising a nucleotide sequence of any one of SEQ ID NOs: 2001-2008, 2010-2035, 2037-2039, 2041, 2044, 2045, 2046, 2048-2051, 2053-2055, 2058, 2060, 2064-2102, 2116, 2117, 2119, 2121-2124, 2126-2129, 2130-2132, 2136, 2137, 2139, 2140, 2142-2156, 2158, 2159, 2161-2169, 2183, 2184, 2188-2202, 2207-2240, 2242, 2243, 2245-2256, 2258-2260, 2266-2281, 2283-2297, 2299-2310, 2340-2355, 2453, 2454, 2456-2458, 2461, 2462, 2464-2466, 2469, 2471, 2475, 2502-2504, 2506-2508, 2521, 2525, 2527, 2528, 2530, 2532-2537, 2575, 2633-2638, 2641-2658, 2663-2680, 2682-2684, 2697-2705, 2718, 2796-2814, 2816, 2817, 2819, 2836-2838, and 2840-2843.

In exemplary aspects, the polypeptide of the invention is further modified to include additional or alternative chemical moieties. For example, the polypeptide of the invention may be glycosylated, amidated, carboxylated, phosphorylated, esterified, N-acylated, cyclized via, e.g., a disulfide bridge, or converted into an acid addition salt and/or optionally dimerized or polymerized, or conjugated.

The polypeptides of the invention (e.g., the fusion polypeptides) can be obtained by methods known in the art. Suitable methods of de novo synthesizing peptides are described in, for example, Chan et al., Fmoc Solid Phase Peptide Synthesis, Oxford University Press, Oxford, United Kingdom, 2005; Peptide and Protein Drug Analysis, ed. Reid, R., Marcel Dekker, Inc., 2000; Epitope Mapping, ed. Westwood et al., Oxford University Press, Oxford, United Kingdom, 2000; and U.S. Pat. No. 5,449,752.

In some embodiments, the polypeptides described herein are commercially synthesized by companies, such as Synpep (Dublin, Calif.), Peptide Technologies Corp. (Gaithersburg, Md.), and Multiple Peptide Systems (San Diego, Calif.). In this respect, the peptides can be synthetic, recombinant, isolated, and/or purified.

Also, in the instances in which the polypeptides do not comprise any non-coded or non-natural amino acids, the polypeptides can be recombinantly produced using a nucleic acid encoding the amino acid sequence of the polypeptides using standard recombinant methods. See, for instance, Sambrook et al., Molecular Cloning: A Laboratory Manual. 3rd ed., Cold Spring Harbor Press, Cold Spring Harbor, N.Y. 2001; and Ausubel et al., Current Protocols in Molecular Biology, Greene Publishing Associates and John Wiley & Sons, N Y, 1994.

In some embodiments, the polypeptides are isolated. The term “isolated” as used herein means having been removed from its natural environment. In exemplary embodiments, the polypeptide is made through recombinant methods and the polypeptide is isolated from the host cell.

In some embodiments, the polypeptides are present in a composition and the composition comprises a purified polypeptide of the invention. The term “purified,” as used herein relates to the isolation of a molecule or compound in a form that is substantially free of contaminants which in some aspects are normally associated with the molecule or compound in a native or natural environment and means having been increased in purity as a result of being separated from other components of the original composition. The purified polypeptides include, for example, peptides substantially free of nucleic acid molecules, lipids, and carbohydrates, or other starting materials or intermediates which are used or formed during chemical synthesis of the peptides. It is recognized that “purity” is a relative term, and not to be necessarily construed as absolute purity or absolute enrichment or absolute selection. In some aspects, the purity is at least or about 50%, is at least or about 60%, at least or about 70%, at least or about 80%, or at least or about 90% (e.g., at least or about 91%, at least or about 92%, at least or about 93%, at least or about 94%, at least or about 95%, at least or about 96%, at least or about 97%, at least or about 98%, at least or about 99% or is approximately 100%.

Nucleic Acid Molecules Encoding Fusion Transcripts

The invention provides isolated nucleic acid molecules comprising a nucleotide sequence of novel fusion genes generated by genomic rearrangements that fuse domains from two distinct genes, and portions thereof, optionally, wherein the portion comprises the junction between the two genes. In exemplary aspects, the nucleic acid molecule comprises the nucleotide sequence (e.g., DNA sequence) of the full length fusion gene, including coding and non-coding sequence. In exemplary aspects, the nucleic acid molecule comprises untranslated regions of a gene, e.g., 5′ untranslated regions (5′ UTR), 3′ untranslated regions (3′ UTR), intronic sequences, and the like. In exemplary aspects, the nucleic acid molecule comprises one or more translated regions of a gene, e.g., exons. In exemplary aspects, the nucleic acid molecule comprises the nucleotide sequence of only the coding sequence of the fusion gene. In exemplary aspects, the coding sequence encodes a transcript, e.g. an RNA transcript. In exemplary aspects, the transcript comprises fused domains encoded by two distinct genes and, in such aspects, the transcript is referenced herein as a “fusion transcript” or a “fusion gene transcript”. Provided herein are nucleic acid molecules encoding any one of the fusion transcripts described herein.

In exemplary aspects, the nucleic acid molecule of the invention comprises a general structure A-B, wherein structure A is a portion of a gene listed in Column A of Table 1 and structure B is a portion of a gene listed in Column B of Table 1, wherein the gene listed in Column A and the gene listed in Column B are listed in the same row of Table 1, wherein structure B is located immediately 3′ to structure A.

In exemplary aspects, the nucleic acid molecule comprises a general structure A-B, wherein structure A is a portion of a gene listed in Column A of Table 1 and structure B is a portion of a gene listed in Column B of Table 1, wherein the gene listed in Column A and the gene listed in Column B are listed in the same row of Table 1 and the row is (a) marked with an asterisk in the 2^{nd} column from the left, (b) not marked with a “#” in the 3^{rd} column from the left, (c) not marked with a “̂” in the 4^{th} column from the left, or (d) a combination thereof, wherein structure B is located immediately 3′ to structure A.

In exemplary aspects, the nucleic acid molecule comprises a general structure A-B, wherein structure A is a portion of a gene listed in Column A of Table 2 and structure B is a portion of a gene listed in Column B of Table 2, wherein the gene listed in Column A and the gene listed in Column B are listed in the same row of Table 2, wherein structure B is located immediately 3′ to structure A. In exemplary aspects, the nucleic acid molecule comprises a general structure A-B, wherein structure A is a portion of a gene listed in Column A of Table 3 and structure B is a portion of a gene listed in Column B of Table 3, wherein the gene listed in Column A and the gene listed in Column B are listed in the same row of Table 3, wherein structure B is located immediately 3′ to structure A. In exemplary aspects, the nucleic acid molecule comprises a general structure A-B, wherein structure A is a portion of a gene listed in Column A of Table 4 and structure B is a portion of a gene listed in Column B of Table 4, wherein the gene listed in Column A and the gene listed in Column B are listed in the same row of Table 4, wherein structure B is located immediately 3′ to structure A.

In exemplary embodiments, the nucleic acid molecule comprises a nucleotide sequence of any one of SEQ ID NOs: 1 to 799. In exemplary embodiments, the nucleic acid molecule comprises a nucleotide sequence of any one of SEQ ID NOs: 800 to 844. In exemplary aspects, the nucleic acid molecule comprises a nucleotide sequence of any one of the SEQ ID NOs: listed in the 9^{th} column from the left of Table 1. In exemplary aspects, the nucleic acid molecule comprises a nucleotide sequence of any one of the SEQ ID NOs: listed in the 9^{th} column from the left of Table 1 in a row (a) marked with a “*” in the 2^{nd} column to the left of Table 1, (b) not marked with a “#” in the 3rd column to the left of Table 1, (c) not marked with a “̂” in the 4th column to the left of Table 1, or (d) a combination thereof.

In exemplary embodiments, the nucleic acid molecule comprises a nucleotide sequence of any one of SEQ ID NOs: 1001-1844. In exemplary aspects, the nucleic acid molecule comprises a nucleotide sequence of any one of the SEQ ID NOs: listed in the 2^{nd} column from the right of Table 1 in a row (a) marked with a “*” in the 2^{nd} column to the left of Table 1, (b) not marked with a “#” in the 3rd column to the left of Table 1, (c) not marked with a “̂” in the 4th column to the left of Table 1, or (d) a combination thereof.

In exemplary embodiments, the nucleic acid molecule comprises a nucleotide sequence encoding any one of SEQ ID NOs: 2001 to 2844. In exemplary aspects, the nucleic acid molecule comprises a nucleotide sequence of any one of the SEQ ID NOs: listed in the right most column of Table 1. In exemplary aspects, the nucleic acid molecule comprises a nucleotide sequence of any one of the SEQ ID NOs: listed in the right most column of Table 1 in a row (a) marked with a “*” in the 2^{nd} column to the left of Table 1, (b) not marked with a “#” in the 3rd column to the left of Table 1, (c) not marked with a “̂” in the 4th column to the left of Table 1, or (d) a combination thereof.

Nucleic acid molecules which are related to the above nucleic acid molecules comprising the aforementioned SEQ ID NOs: are provided. For example, nucleic acid molecules which are degenerate to the above nucleic acid molecules comprising the aforementioned SEQ ID NOs: and nucleic acid molecules which are complements of the above nucleic acid molecules comprising the aforementioned SEQ ID NOs: are provided.

In exemplary aspects, the nucleic acid molecules described herein are isolated. In exemplary aspects, the nucleic acid molecules of the invention exist in a composition and the composition has a given % purity with regard to the nucleic acid molecule. For example, the purity can be at least about 50%, can be greater than 60%, 70% or 80%, or can be 100%.

The nucleic acid molecules in some aspects are single stranded and in other aspects are double stranded. The nucleic acid molecules may be modified to comprise additional functional or chemical moieties, such as, for example, a detectable label. The detectable label can be, for instance, a radioisotope, a fluorophore, and an element particle.

By “nucleic acid molecule” as used herein includes “polynucleotide,” “oligonucleotide,” and “nucleic acid,” and generally means a polymer of DNA or RNA, which can be single-stranded or double-stranded, synthesized or obtained (e.g., isolated and/or purified) from natural sources, which can contain natural, non-natural or altered nucleotides, and which can contain a natural, non-natural or altered internucleotide linkage, such as a phosphoroamidate linkage or a phosphorothioate linkage, instead of the phosphodiester found between the nucleotides of an unmodified oligonucleotide. It is generally preferred that the nucleic acid does not comprise any insertions, deletions, inversions, and/or substitutions. However, it may be suitable in some instances, as discussed herein, for the nucleic acid to comprise one or more insertions, deletions, inversions, and/or substitutions.

In some aspects, the nucleic acids of the invention are recombinant. As used herein, the term “recombinant” refers to (i) molecules that are constructed outside living cells by joining natural or synthetic nucleic acid segments to nucleic acid molecules that can replicate in a living cell, or (ii) molecules that result from the replication of those described in (i) above. For purposes herein, the replication can be in vitro replication or in vivo replication.

The nucleic acids can be constructed based on chemical synthesis and/or enzymatic ligation reactions using procedures known in the art. See, for example, Sambrook et al., supra, and Ausubel et al., supra. For example, a nucleic acid can be chemically synthesized using naturally occurring nucleotides or variously modified nucleotides designed to increase the biological stability of the molecules or to increase the physical stability of the duplex formed upon hybridization (e.g., phosphorothioate derivatives and acridine substituted nucleotides). Examples of modified nucleotides that can be used to generate the nucleic acids include, but are not limited to, 5-fluorouracil, 5-bromouracil, 5-chlorouracil, 5-iodouracil, hypoxanthine, xanthine, 4-acetylcytosine, 5-(carboxyhydroxymethyl) uracil, 5-carboxymethylaminomethyl-2-thiouridme, 5-carboxymethylaminomethyluracil, dihydrouracil, beta-D-galactosylqueosine, inosine, N⁶-isopentenyladenine, 1-methylguanine, 1-methylinosine, 2,2-dimethylguanine, 2-methyladenine, 2-methylguanine, 3-methylcytosine, 5-methylcytosine, N-substituted adenine, 7-methylguanine, 5-methylammomethyluracil, 5-methoxyaminomethyl-2-thiouracil, beta-D-mannosylqueosine, 5′-methoxycarboxymethyluracil, 5-methoxyuracil, 2-methylthio-N⁶-isopentenyladenine, uracil-5-oxyacetic acid (v), wybutoxosine, pseudouratil, queosine, 2-thiocytosine, 5-methyl-2-thiouracil, 2-thiouracil, 4-thiouracil, 5-methyluracil, uracil-5-oxyacetic acid methylester, 3-(3-amino-3-N-2-carboxypropyl) uracil, and 2,6-diaminopurine. Alternatively, one or more of the nucleic acids of the invention can be purchased from companies, such as Macromolecular Resources (Fort Collins, Colo.) and Synthegen (Houston, Tex.).

Recombinant Expression Vector

The nucleic acids of the invention in exemplary aspects are incorporated into a recombinant expression vector. In this regard, the invention provides recombinant expression vectors comprising any of the nucleic acids described herein. For purposes herein, the term “recombinant expression vector” means a genetically-modified oligonucleotide or polynucleotide construct that permits the expression of an mRNA, protein, polypeptide, or peptide by a host cell, when the construct comprises a nucleotide sequence encoding the mRNA, protein, polypeptide, or peptide, and the vector is contacted with the cell under conditions sufficient to have the mRNA, protein, polypeptide, or peptide expressed within the cell. The vectors of the invention are not naturally-occurring as a whole. However, parts of the vectors may be naturally-occurring. The inventive recombinant expression vectors may comprise any type of nucleotides, including, but not limited to DNA and RNA, which may be single-stranded or double-stranded, synthesized or obtained in part from natural sources, and which may contain natural, non-natural or altered nucleotides. The recombinant expression vectors may comprise naturally-occurring or non-naturally-occuring internucleotide linkages, or both types of linkages. In exemplary aspects, the altered nucleotides or non-naturally occurring internucleotide linkages do not hinder the transcription or replication of the vector.

The recombinant expression vector of the invention may be any suitable recombinant expression vector, and may be used to transform or transfect any suitable host. Suitable vectors include those designed for propagation and expansion or for expression or both, such as plasmids and viruses. The vector may be selected from the group consisting of the pUC series (Fermentas Life Sciences), the pBluescript series (Stratagene, LaJolla, Calif.), the pET series (Novagen, Madison, Wis.), the pGEX series (Pharmacia Biotech, Uppsala, Sweden), and the pEX series (Clontech, Palo Alto, Calif.). Bacteriophage vectors, such as λGTIO, λGTI 1, λZapII (Stratagene), λEMBL4, and λNMI 149, also may be used. Examples of plant expression vectors include pBIOI, pBI101.2, pBI101.3, pBI121 and pBIN19 (Clontech). Examples of animal expression vectors include pEUK-Cl, pMAM and pMAMneo (Clontech). In exemplary aspects, the recombinant expression vector is a viral vector, e.g., a retroviral vector.

The recombinant expression vectors of the invention may be prepared using standard recombinant DNA techniques described in, for example, Sambrook et al., supra, and Ausubel et al., supra. Constructs of expression vectors, which are circular or linear, may be prepared to contain a replication system functional in a prokaryotic or eukaryotic host cell. Replication systems may be derived, e.g., from ColEl, 2μ plasmid, λ, SV40, bovine papilloma virus, and the like.

In exemplary aspects, the recombinant expression vector comprises regulatory sequences, such as transcription and translation initiation and termination codons, which are specific to the type of host (e.g., bacterium, fungus, plant, or animal) into which the vector is to be introduced, as appropriate and taking into consideration whether the vector is DNA- or RNA-based.

The recombinant expression vector may include one or more marker genes, which allow for selection of transformed or transfected hosts. Marker genes include biocide resistance, e.g., resistance to antibiotics, heavy metals, etc., complementation in an auxotrophic host to provide prototrophy, and the like. Suitable marker genes for the inventive expression vectors include, for instance, neomycin/G418 resistance genes, hygromycin resistance genes, histidinol resistance genes, tetracycline resistance genes, and ampicillin resistance genes.

The recombinant expression vector may comprise a native or normative promoter operably linked to the nucleotide sequence encoding the binding agent or conjugate or to the nucleotide sequence which is complementary to or which hybridizes to the nucleotide sequence encoding the binding agent or conjugate. The selection of promoters, e.g., strong, weak, inducible, tissue-specific and developmental-specific, is within the ordinary skill of the artisan.

Similarly, the combining of a nucleotide sequence with a promoter is also within the skill of the artisan. The promoter may be a non-viral promoter or a viral promoter, e.g., a cytomegalovirus (CMV) promoter, an SV40 promoter, an RSV promoter, and a promoter found in the long-terminal repeat of the murine stem cell virus.

The inventive recombinant expression vectors may be designed for either transient expression, for stable expression, or for both. Also, the recombinant expression vectors may be made for constitutive expression or for inducible expression. Further, the recombinant expression vectors may be made to include a suicide gene.

As used herein, the term “suicide gene” refers to a gene that causes the cell expressing the suicide gene to die. The suicide gene may be a gene that confers sensitivity to an agent, e.g., a drug, upon the cell in which the gene is expressed, and causes the cell to die when the cell is contacted with or exposed to the agent. Suicide genes are known in the art (see, for example, Suicide Gene Therapy: Methods and Reviews. Springer, Caroline J. (Maycer Research UK Centre for Maycer Therapeutics at the Institute of Maycer Research, Sutton, Surrey, UK), Humana Press, 2004) and include, for example, the Herpes Simplex Virus (HSV) thymidine kinase (TK) gene, cytosine daminase, purine nucleoside phosphorylase, and nitroreductase.

Host Cells

The invention further provides a host cell comprising any of the nucleic acids or vectors described herein. As used herein, the term “host cell” refers to any type of cell that may contain the nucleic acid or vector described herein. In exemplary aspects, the host cell is a eukaryotic cell, e.g., plant, animal, fungi, or algae, or may be a prokaryotic cell, e.g., bacteria or protozoa. In exemplary aspects, the host cells is a cell originating or obtained from a subject, as described herein. In exemplary aspects, the host cell originates from or is obtained from a mammal. As used herein, the term “mammal” refers to any mammal, including, but not limited to, mammals of the order Rodentia, such as mice and hamsters, and mammals of the order Logomorpha, such as rabbits. It is preferred that the mammals are from the order Carnivora, including Felines (cats) and Canines (dogs). It is more preferred that the mammals are from the order Artiodactyla, including Bo vines (cows) and S wines (pigs) or of the order Perssodactyla, including Equines (horses). It is most preferred that the mammals are of the order Primates, Ceboids, or Simoids (monkeys) or of the order Anthropoids (humans and apes). An especially preferred mammal is the human.

In exemplary aspects, the host cell is a cultured cell or a primary cell, i.e., isolated directly from an organism, e.g., a human. The host cell in exemplary aspects is an adherent cell or a suspended cell, i.e., a cell that grows in suspension. Suitable host cells are known in the art and include, for instance, DH5? E. coli cells, Chinese hamster ovarian (CHO) cells, monkey VERO cells, T293 cells, COS cells, HEK293 cells, and the like. For purposes of amplifying or replicating the recombinant expression vector, the host cell is preferably a prokaryotic cell, e.g., a DH5a cell. In exemplary aspects, the host cell is a human cell. The host cell may be of any cell type, may originate from any type of tissue, and may be of any developmental stage.

Also provided by the invention is a population of cells comprising at least one host cell described herein. The population of cells may be a heterogeneous population comprising the host cell comprising any of the expression vectors described, in addition to at least one other cell, e.g., a host cell, which does not comprise any of the recombinant expression vectors. Alternatively, the population of cells may be a substantially homogeneous population, in which the population comprises mainly of host cells (e.g., consisting essentially of) comprising the expression vector. The population also may be a clonal population of cells, in which all cells of the population are clones of a single host cell comprising a recombinant expression vector, such that all cells of the population comprise the recombinant expression vector. In exemplary embodiments of the invention, the population of cells is a clonal population comprising host cells expressing a nucleic acid or a vector described herein.

Binding Agents

Binding Agents: Antibodies

The invention provides binding agents which specifically bind to a polypeptide of the invention. In exemplary aspects, the binding agent is an antibody, an antigen binding fragment thereof, or an antibody derivative, wherein the antibody, antigen binding fragment thereof or antibody derivative comprises six complementarity determining regions. In exemplary aspects, the binding agent specifically binds to an epitope comprising a junction of the fusion polypeptide. The junctions of the fusion polypeptides are described in Table 5 by way of providing the location of the junction in the cDNA of the fusion transcripts.

In exemplary aspects, the antibody can be any type of immunoglobulin that is known in the art. For instance, the antibody can be of any isotype, e.g., IgA, IgD, IgE, IgG, IgM. The antibody can be monoclonal or polyclonal. The antibody can be a naturally-occurring antibody, i.e., an antibody isolated and/or purified from a mammal, e.g., mouse, rabbit, goat, horse, chicken, hamster, human, and the like. In this regard, the antibody may be considered to be a mammalian antibody, e.g., a mouse antibody, rabbit antibody, goat antibody, horse antibody, chicken antibody, hamster antibody, human antibody, and the like.

In exemplary aspects, the antibody is considered to be a blocking antibody or neutralizing antibody. In exemplary aspects, the antibody is not a blocking antibody or neutralizing antibody.

In exemplary aspects, the dissocation constant (K_{D}) of the antibody for the polypeptide of the invention is between about 0.0001 nM and about 100 nM. In some embodiments, the K_{D} is at least or about 0.0001 nM, at least or about 0.001 nM, at least or about 0.01 nM, at least or about 0.1 nM, at least or about 1 nM, or at least or about 10 nM. In some embodiments, the K_{D} is no more than or about 100 nM, no more than or about 75 nM, no more than or about 50 nM, or no more than or about 25 nM.

In exemplary embodiments, the antibody is a genetically engineered antibody, e.g., a single chain antibody, a humanized antibody, a chimeric antibody, a CDR-grafted antibody, an antibody that includes portions of CDR sequences specific for the polypeptide of the invention, a humaneered antibody, a bispecific antibody, a trispecific antibody, and the like. Genetic engineering techniques also provide the ability to make fully human antibodies in a non-human.

In some aspects, the antibody is a chimeric antibody. The term “chimeric antibody” is used herein to refer to an antibody containing constant domains from one species and the variable domains from a second, or more generally, containing stretches of amino acid sequence from at least two species.

In some aspects, the antibody is a humanized antibody. The term “humanized” when used in relation to antibodies is used to refer to antibodies having at least CDR regions from a nonhuman source that are engineered to have a structure and immunological function more similar to true human antibodies than the original source antibodies. For example, humanizing can involve grafting CDR from a non-human antibody, such as a mouse antibody, into a human antibody. Humanizing also can involve select amino acid substitutions to make a non-human sequence look more like a human sequence, as would be known in the art.

Use of the terms “chimeric or humanized” herein is not meant to be mutually exclusive; rather, is meant to encompass chimeric antibodies, humanized antibodies, and chimeric antibodies that have been further humanized. Except where context otherwise indicates, statements about (properties of, uses of, testing, and so on) chimeric antibodies apply to humanized antibodies, and statements about humanized antibodies pertain also to chimeric antibodies. Likewise, except where context dictates, such statements also should be understood to be applicable to antibodies and antigen binding fragments of such antibodies.

In some aspects of the disclosure, the binding agent is an antigen binding fragment of an antibody that specifically binds to a polypeptide in accordance with the invention. The antigen binding fragment (also referred to herein as “antigen binding portion”) may be an antigen binding fragment of any of the antibodies described herein. The antigen binding fragment can be any part of an antibody that has at least one antigen binding site, including, but not limited to, Fab, F(ab′)₂, dsFv, sFv, diabodies, triabodies, bis-scFvs, fragments expressed by a Fab expression library, domain antibodies, VhH domains, V-NAR domains, VH domains, VL domains, and the like. Antibody fragments of the invention, however, are not limited to these exemplary types of antibody fragments.

In exemplary aspects, the antigen binding fragment is a domain antibody. A domain antibody comprises a functional binding unit of an antibody, and can correspond to the variable regions of either the heavy (V_{H}) or light (V_{L}) chains of antibodies. A domain antibody can have a molecular weight of approximately 13 kDa, or approximately one-tenth the weight of a full antibody. Domain antibodies may be derived from full antibodies, such as those described herein. The antigen binding fragments in some embodiments are monomeric or polymeric, bispecific or trispecific, and bivalent or trivalent.

Antibody fragments that contain the antigen binding, or idiotope, of the antibody molecule share a common idiotype and are contemplated by the disclosure. Such antibody fragments may be generated by techniques known in the art and include, but are not limited to, the F(ab′)₂ fragment which may be produced by pepsin digestion of the antibody molecule; the Fab′ fragments which may be generated by reducing the disulfide bridges of the F(ab′)₂ fragment, and the two Fab′ fragments which may be generated by treating the antibody molecule with papain and a reducing agent.

In exemplary aspects, the binding agent provided herein is a single-chain variable region fragment (scFv) antibody fragment. An scFv may consist of a truncated Fab fragment comprising the variable (V) domain of an antibody heavy chain linked to a V domain of an antibody light chain via a synthetic peptide, and it can be generated using routine recombinant DNA technology techniques (see, e.g., Janeway et al., Immunobiology, 2^{nd} Edition, Garland Publishing, New York, (1996)). Similarly, disulfide-stabilized variable region fragments (dsFv) can be prepared by recombinant DNA technology (see, e.g., Reiter et al., Protein Engineering, 7, 697-704 (1994)).

Recombinant antibody fragments, e.g., scFvs of the disclosure, can also be engineered to assemble into stable multimeric oligomers of high binding avidity and specificity to different target antigens. Such diabodies (dimers), triabodies (trimers) or tetrabodies (tetramers) are well known in the art. See e.g., Kortt et al., Biomol Eng. 2001 18:95-108, (2001) and Todorovska et al., J Immunol Methods. 248:47-66, (2001).

In exemplary aspects, the binding agent is a bispecific antibody (bscAb). Bispecific antibodies are molecules comprising two single-chain Fv fragments joined via a glycine-serine linker using recombinant methods. The V light-chain (V_{L}) and V heavy-chain (V_{H}) domains of two antibodies of interest in exemplary embodiments are isolated using standard PCR methods. The V_{L} and V_{H} cDNAs obtained from each hybridoma are then joined to form a single-chain fragment in a two-step fusion PCR. Bispecific fusion proteins are prepared in a similar manner. Bispecific single-chain antibodies and bispecific fusion proteins are antibody substances included within the scope of the present invention. Exemplary bispecific antibodies are taught in U.S. Patent Application Publication No. 2005-0282233A1 and International Patent Application Publication No. WO 2005/087812, both applications of which are incorporated herein by reference in their entireties.

In exemplary aspects, the binding agent is a bispecific T-cell engaging antibody (BiTE) containing two scFvs produced as a single polypeptide chain. Methods of making and using BiTE antibodies are described in the art. See, e.g., Cioffi et al., Clin Cancer Res 18: 465, Brischwein et al., Mol Immunol 43:1129-43 (2006); Amann M et al., Cancer Res 68:143-51 (2008); Schlereth et al., Cancer Res 65: 2882-2889 (2005); and Schlereth et al., Cancer Immunol Immunother 55:785-796 (2006).

In exemplary aspects, the binding agent is a dual affinity re-targeting antibody (DART). DARTs are produced as separate polypeptides joined by a stabilizing interchain disulphide bond. Methods of making and using DART antibodies are described in the art. See, e.g., Rossi et al., MAbs 6: 381-91 (2014); Fournier and Schirrmacher, BioDrugs 27:35-53 (2013); Johnson et al., J Mol Biol 399:436-449 (2010); Brien et al., J Virol 87: 7747-7753 (2013); and Moore et al., Blood 117:4542 (2011).

In exemplary aspects, the binding agent is a tetravalent tandem diabody (TandAbs) in which an antibody fragment is produced as a non covalent homodimer folder in a head-to-tail arrrangement. TandAbs are known in the art. See, e.g., McAleese et al., Future Oncol 8: 687-695 (2012); Portner et al., Cancer Immunol Immunother 61:1869-1875 (2012); and Reusch et al., MAbs 6:728 (2014).

In exemplary aspects, the BiTE, DART, or TandAbs comprises the CDRs of any one of the antibodies described herein.

Suitable methods of making antibodies are known in the art. For instance, standard hybridoma methods are described in, e.g., Harlow and Lane (eds.), Antibodies: A Laboratory Manual, CSH Press (1988), and CA. Janeway et al. (eds.), Immunobiology, 5^{th} Ed., Garland Publishing, New York, N.Y. (2001)).

Monoclonal antibodies for use in the invention may be prepared using any technique that provides for the production of antibody molecules by continuous cell lines in culture. These include, but are not limited to, the hybridoma technique originally described by Koehler and Milstein (Nature 256: 495-497, 1975), the human B-cell hybridoma technique (Kosbor et al., Immunol Today 4:72, 1983; Cote et al., Proc Natl Acad Sci 80: 2026-2030, 1983) and the EBV-hybridoma technique (Cole et al., Monoclonal Antibodies and Cancer Therapy, Alan R Liss Inc, New York N.Y., pp 77-96, (1985).

Briefly, a polyclonal antibody is prepared by immunizing an animal with an immunogen comprising a polypeptide of the present invention and collecting antisera from that immunized animal. A wide range of animal species can be used for the production of antisera. In some aspects, an animal used for production of anti-antisera is a non-human animal including rabbits, mice, rats, hamsters, goat, sheep, pigs or horses. Because of the relatively large blood volume of rabbits, a rabbit, in some exemplary aspects, is a preferred choice for production of polyclonal antibodies. In an exemplary method for generating a polyclonal antisera immunoreactive with the chosen epitope, 50 μg of polypeptide antigen is emulsified in Freund's Complete Adjuvant for immunization of rabbits. At intervals of, for example, 21 days, 50 μg of epitope are emulsified in Freund's Incomplete Adjuvant for boosts. Polyclonal antisera may be obtained, after allowing time for antibody generation, simply by bleeding the animal and preparing serum samples from the whole blood.

Briefly, in exemplary embodiments, to generate monoclonal antibodies, a mouse is injected periodically with recombinant polypeptide against which the antibody is to be raised (e.g., 10-20 μg polypeptide emulsified in Freund's Complete Adjuvant). The mouse is given a final pre-fusion boost of a polypeptide containing the epitope that allows specific recognition of lymphatic endothelial cells in PBS, and four days later the mouse is sacrificed and its spleen removed. The spleen is placed in 10 ml serum-free RPMI 1640, and a single cell suspension is formed by grinding the spleen between the frosted ends of two glass microscope slides submerged in serum-free RPMI 1640, supplemented with 2 mM L-glutamine, 1 mM sodium pyruvate, 100 units/ml penicillin, and 100 μg/ml streptomycin (RPMI) (Gibco, Canada). The cell suspension is filtered through sterile 70-mesh Nitex cell strainer (Becton Dickinson, Parsippany, N.J.), and is washed twice by centrifuging at 200 g for 5 minutes and resuspending the pellet in 20 ml serum-free RPMI. Splenocytes taken from three naive Balb/c mice are prepared in a similar manner and used as a control. NS-1 myeloma cells, kept in log phase in RPMI with 11% fetal bovine serum (FBS) (Hyclone Laboratories, Inc., Logan, Utah) for three days prior to fusion, are centrifuged at 200 g for 5 minutes, and the pellet is washed twice.

Spleen cells (1×10⁸) are combined with 2.0×10⁷ NS-1 cells and centrifuged, and the supernatant is aspirated. The cell pellet is dislodged by tapping the tube, and 1 ml of 37° C. PEG 1500 (50% in 75 mM Hepes, pH 8.0) (Boehringer Mannheim) is added with stirring over the course of 1 minute, followed by the addition of 7 ml of serum-free RPMI over 7 minutes. An additional 8 ml RPMI is added and the cells are centrifuged at 200 g for 10 minutes. After discarding the supernatant, the pellet is resuspended in 200 ml RPMI containing 15% FBS, 100 μM sodium hypoxanthine, 0.4 μM aminopterin, 16 μM thymidine (HAT) (Gibco), 25 units/ml IL-6 (Boehringer Mannheim) and 1.5×10⁶ splenocytes/ml and plated into 10 Corning flat-bottom 96-well tissue culture plates (Corning, Corning N.Y.).

On days 2, 4, and 6, after the fusion, 100 μl of medium is removed from the wells of the fusion plates and replaced with fresh medium. On day 8, the fusion is screened by ELISA, testing for the presence of mouse IgG binding to polypeptide as follows. Immulon 4 plates (Dynatech, Cambridge, Mass.) are coated for 2 hours at 37° C. with 100 ng/well of ID 3Rα2 diluted in 25 mM Tris, pH 7.5. The coating solution is aspirated and 200 μl/well of blocking solution (0.5% fish skin gelatin (Sigma) diluted in CMF-PBS) is added and incubated for 30 minutes at 37° C. Plates are washed three times with PBS containing 0.05% Tween 20 (PBST) and 50 μl culture supernatant is added. After incubation at 37° C. for 30 minutes, and washing as above, 50 μl of horseradish peroxidase-conjugated goat anti-mouse IgG(Fc) (Jackson ImmunoResearch, West Grove, Pa.) diluted 1:3500 in PBST is added. Plates are incubated as above, washed four times with PBST, and 100 μl substrate, consisting of 1 mg/ml o-phenylene diamine (Sigma) and 0.1 μl/ml 30% H₂O₂ in 100 mM citrate, pH 4.5, are added. The color reaction is stopped after 5 minutes with the addition of 50 μl of 15% H₂SO₄. The A₄₉₀ absorbance is determined using a plate reader (Dynatech).

Selected fusion wells are cloned twice by dilution into 96-well plates and visual scoring of the number of colonies/well after 5 days. The monoclonal antibodies produced by hybridomas are isotyped using the Isostrip system (Boehringer Mannheim, Indianapolis, Ind.).

When the hybridoma technique is employed, myeloma cell lines may be used. Such cell lines suited for use in hybridoma-producing fusion procedures preferably are non-antibody-producing, have high fusion efficiency, and enzyme deficiencies that render them incapable of growing in certain selective media that support the growth of only the desired fused cells (hybridomas). For example, where the immunized animal is a mouse, one may use P3-X63/Ag8, P3-X63-Ag8.653, NS1/1.Ag 4 1, Sp210-Ag14, FO, NSO/U, MPC-11, MPC11-X45-GTG 1.7 and S194/15XX0 Bul; for rats, one may use R210.RCY3, Y3-Ag 1.2.3, IR983F and 4B210; and U-266, GM1500-GRG2, LICR-LON-HMy2 and UC729-6 are all useful in connection with cell fusions. It should be noted that the hybridomas and cell lines produced by such techniques for producing the monoclonal antibodies are contemplated to be compositions of the disclosure.

Depending on the host species, various adjuvants may be used to increase an immunological response. Such adjuvants include, but are not limited to, Freund's, mineral gels such as aluminum hydroxide, and surface active substances such as lysolecithin, pluronic polyols, polyanions, peptides, oil emulsions, keyhole limpet hemocyanin, and dinitrophenol. BCG (bacilli Calmette-Guerin) and Corynebacterium parvum are potentially useful human adjuvants.

Alternatively, other methods, such as EBV-hybridoma methods (Haskard and Archer, J. Immunol. Methods, 74(2), 361-67 (1984), and Roder et al.₅ Methods Enzymol., 121, 140-67 (1986)), and bacteriophage vector expression systems (see, e.g., Huse et al., Science, 246, 1275-81 (1989)) that are known in the art may be used. Further, methods of producing antibodies in non-human animals are described in, e.g., U.S. Pat. Nos. 5,545,806, 5,569,825, and 5,714,352, and U.S. Patent Application Publication No. 2002/0197266 A1).

Antibodies may also be produced by inducing in vivo production in the lymphocyte population or by screening recombinant immunoglobulin libraries or panels of highly specific binding reagents as disclosed in Orlandi et al. (Proc. Natl. Acad. Sci. 86: 3833-3837; 1989), and Winter and Milstein (Nature 349: 293-299, 1991).

Furthermore, phage display can be used to generate an antibody of the disclosure. In this regard, phage libraries encoding antigen-binding variable (V) domains of antibodies can be generated using standard molecular biology and recombinant DNA techniques (see, e.g., Sambrook et al. (eds.), Molecular Cloning, A Laboratory Manual, 3^{rd} Edition, Cold Spring Harbor Laboratory Press, New York (2001)). Phage encoding a variable region with the desired specificity are selected for specific binding to the desired antigen, and a complete or partial antibody is reconstituted comprising the selected variable domain. Nucleic acid sequences encoding the reconstituted antibody are introduced into a suitable cell line, such as a myeloma cell used for hybridoma production, such that antibodies having the characteristics of monoclonal antibodies are secreted by the cell (see, e.g., Janeway et al., supra, Huse et al., supra, and U.S. Pat. No. 6,265,150). Related methods also are described in U.S. Pat. Nos. 5,403,484; 5,571,698; 5,837,500; and 5,702,892. The techniques described in U.S. Pat. Nos. 5,780,279; 5,821,047; 5,824,520; 5,855,885; 5,858,657; 5,871,907; 5,969,108; 6,057,098; and 6,225,447, are also contemplated as useful in preparing antibodies according to the disclosure.

Antibodies can be produced by transgenic mice that are transgenic for specific heavy and light chain immunoglobulin genes. Such methods are known in the art and described in, for example U.S. Pat. Nos. 5,545,806 and 5,569,825, and Janeway et al., supra.

Methods for generating humanized antibodies are well known in the art and are described in detail in, for example, Janeway et al., supra, U.S. Pat. Nos. 5,225,539; 5,585,089; and 5,693,761; European Patent No. 0239400 BI; and United Kingdom Patent No. 2188638. Humanized antibodies can also be generated using the antibody resurfacing technology described in U.S. Pat. No. 5,639,641 and Pedersen et al., J. Mol. Biol., 235:959-973 (1994).

Techniques developed for the production of “chimeric antibodies,” the splicing of mouse antibody genes to human antibody genes to obtain a molecule with appropriate antigen specificity and biological activity, can be used (Morrison et al., Proc. Natl. Acad. Sci. 81: 6851-6855, 1984; Neuberger et al., Nature 312: 604-608, 1984; and Takeda et al., Nature 314: 452-454; 1985). Alternatively, techniques described for the production of single-chain antibodies (U.S. Pat. No. 4,946,778) can be adapted to produce IL13Rα2-specific single chain antibodies.

A preferred chimeric or humanized antibody has a human constant region, while the variable region, or at least a CDR, of the antibody is derived from a non-human species. Methods for humanizing non-human antibodies are well known in the art. (see U.S. Pat. Nos. 5,585,089, and 5,693,762). Generally, a humanized antibody has one or more amino acid residues introduced into a CDR region and/or into its framework region from a source which is non-human. Humanization can be performed, for example, using methods described in Jones et al. (Nature 321: 522-525, 1986), Riechmann et al., (Nature, 332: 323-327, 1988) and Verhoeyen et al. (Science 239:1534-1536, 1988), by substituting at least a portion of a rodent complementarity-determining region (CDR) for the corresponding region of a human antibody. Numerous techniques for preparing engineered antibodies are described, e.g., in Owens and Young, J. Immunol. Meth., 168:149-165 (1994). Further changes can then be introduced into the antibody framework to modulate affinity or immunogenicity.

Consistent with the foregoing description, compositions comprising CDRs may be generated using, at least in part, techniques known in the art to isolate CDRs. Complementarity-determining regions are characterized by six polypeptide loops, three loops for each of the heavy or light chain variable regions. The amino acid position in a CDR is defined by Kabat et al., “Sequences of Proteins of Immunological Interest,” U.S. Department of Health and Human Services, (1983), which is incorporated herein by reference. For example, hypervariable regions of human antibodies are roughly defined to be found at residues 28 to 35, from 49-59 and from residues 92-103 of the heavy and light chain variable regions [Janeway et al., supra]. The murine CDRs also are found at approximately these amino acid residues. It is understood in the art that CDR regions may be found within several amino acids of the approximated amino acid positions set forth above. An immunoglobulin variable region also consists of four “framework” regions surrounding the CDRs (FR1-4). The sequences of the framework regions of different light or heavy chains are highly conserved within a species, and are also conserved between human and murine sequences.

Compositions comprising one, two, and/or three CDRs of a heavy chain variable region or a light chain variable region of a monoclonal antibody are generated. Polypeptide compositions comprising one, two, three, four, five and/or six complementarity-determining regions of an antibody are also contemplated. Using the conserved framework sequences surrounding the CDRs, PCR primers complementary to these consensus framework sequences are generated to amplify the CDR sequence located between the primer regions. Techniques for cloning and expressing nucleotide and polypeptide sequences are well-established in the art [see e.g., Sambrook et al., Molecular Cloning: A Laboratory Manual, 2^{nd} Edition, Cold Spring Harbor, N.Y. (1989)]. The amplified CDR sequences are ligated into an appropriate plasmid. The plasmid comprising one, two, three, four, five and/or six cloned CDRs optionally contains additional polypeptide encoding regions linked to the CDR.

Framework regions (FR) of a murine antibody are humanized by substituting compatible human framework regions chosen from a large database of human antibody variable sequences, including over twelve hundred human V_{H} sequences and over one thousand V_{L} sequences. The database of antibody sequences used for comparison is downloaded from Andrew C. R. Martin's KabatMan web page (http://www.rubic.rdg.ac.uk/abs/). The Kabat method for identifying CDRs provides a means for delineating the approximate CDR and framework regions of any human antibody and comparing the sequence of a murine antibody for similarity to determine the CDRs and FRs. Best matched human V_{H} and V_{L} sequences are chosen on the basis of high overall framework matching, similar CDR length, and minimal mismatching of canonical and V_{H}/V_{L} contact residues. Human framework regions most similar to the murine sequence are inserted between the murine CDRs. Alternatively, the murine framework region may be modified by making amino acid substitutions of all or part of the native framework region that more closely resemble a framework region of a human antibody.

“Conservative” amino acid substitutions are made on the basis of similarity in polarity, charge, solubility, hydrophobicity, hydrophilicity, and/or the amphipathic nature of the residues involved. For example, nonpolar (hydrophobic) amino acids include alanine (Ala, A), leucine (Leu, L), isoleucine (Ile, I), valine (Val, V), proline (Pro, P), phenylalanine (Phe, F), tryptophan (Trp, W), and methionine (Met, M); polar neutral amino acids include glycine (Gly, G), serine (Ser, S), threonine (Thr, T), cysteine (Cys, C), tyrosine (Tyr, Y), asparagine (Asn, N), and glutamine (Gln, Q); positively charged (basic) amino acids include arginine (Arg, R), lysine (Lys, K), and histidine (His, H); and negatively charged (acidic) amino acids include aspartic acid (Asp, D) and glutamic acid (Glu, E). “Insertions” or “deletions” are preferably in the range of about 1 to 20 amino acids, more preferably 1 to 10 amino acids. The variation may be introduced by systematically making substitutions of amino acids in a polypeptide molecule using recombinant DNA techniques and assaying the resulting recombinant variants for activity. Nucleic acid alterations can be made at sites that differ in the nucleic acids from different species (variable positions) or in highly conserved regions (constant regions). Methods for expressing polypeptide compositions useful in the invention are described in greater detail below.

Additionally, another useful technique for generating antibodies for use in the methods of the invention may be one which uses a rational design-type approach. The goal of rational design is to produce structural analogs of biologically active polypeptides or compounds with which they interact (agonists, antagonists, inhibitors, peptidomimetics, binding partners, and the like). By creating such analogs, it is possible to fashion additional antibodies which are more immunoreactive than the native or natural molecule. In one approach, one would generate a three-dimensional structure for the antibodies or an epitope binding fragment thereof. This could be accomplished by x-ray crystallography, computer modeling or by a combination of both approaches. An alternative approach, “alanine scan,” involves the random replacement of residues throughout a molecule with alanine, and the resulting effect on function is determined.

It also is possible to solve the crystal structure of the specific antibodies. In principle, this approach yields a pharmacore upon which subsequent drug design can be based. It is possible to bypass protein crystallography altogether by generating anti-idiotypic antibodies to a functional, pharmacologically active antibody. As a mirror image of a mirror image, the binding site of anti-idiotype antibody is expected to be an analog of the original antigen. The anti-idiotype antibody is then be used to identify and isolate additional antibodies from banks of chemically- or biologically-produced peptides.

Chemically synthesized bispecific antibodies may be prepared by chemically cross-linking heterologous Fab or F(ab′)₂ fragments by means of chemicals such as heterobifunctional reagent succinimidyl-3-(2-pyridyldithiol)-propionate (SPDP, Pierce Chemicals, Rockford, Ill.). The Fab and F(ab′)₂ fragments can be obtained from intact antibody by digesting it with papain or pepsin, respectively (Karpovsky et al., J. Exp. Med. 160:1686-701, 1984; Titus et al., J. Immunol., 138:4018-22, 1987).

Methods of testing antibodies for the ability to bind to the epitope of the polypeptide of the invention, regardless of how the antibodies are produced, are known in the art and include any antibody-antigen binding assay such as, for example, radioimmunoassay (RIA), ELISA, Western blot, immunoprecipitation, and competitive inhibition assays (see, e.g., Janeway et al., infra, and U.S. Patent Application Publication No. 2002/0197266 A1).

Aptamers

Recent advances in the field of combinatorial sciences have identified short polymer sequences (e.g., oligonucleic acid or peptide molecules) with high affinity and specificity to a given target. For example, SELEX technology has been used to identify DNA and RNA aptamers with binding properties that rival mammalian antibodies, the field of immunology has generated and isolated antibodies or antibody fragments which bind to a myriad of compounds, and phage display has been utilized to discover new peptide sequences with very favorable binding properties. Based on the success of these molecular evolution techniques, it is certain that molecules can be created which bind to any target molecule. A loop structure is often involved with providing the desired binding attributes as in the case of aptamers, which often utilize hairpin loops created from short regions without complementary base pairing, naturally derived antibodies that utilize combinatorial arrangement of looped hyper-variable regions and new phage-display libraries utilizing cyclic peptides that have shown improved results when compared to linear peptide phage display results. Thus, sufficient evidence has been generated to indicate that high affinity ligands can be created and identified by combinatorial molecular evolution techniques. For the present disclosure, molecular evolution techniques can be used to isolate binding agents specific for the polypeptide disclosed herein. For more on aptamers, see generally, Gold, L., Singer, B., He, Y. Y., Brody. E., “Aptamers As Therapeutic And Diagnostic Agents,” J. Biotechnol. 74:5-13 (2000). Relevant techniques for generating aptamers are found in U.S. Pat. No. 6,699,843, which is incorporated herein by reference in its entirety.

In some embodiments, the aptamer is generated by preparing a library of nucleic acids; contacting the library of nucleic acids with a growth factor, wherein nucleic acids having greater binding affinity for the growth factor (relative to other library nucleic acids) are selected and amplified to yield a mixture of nucleic acids enriched for nucleic acids with relatively higher affinity and specificity for binding to the growth factor. The processes may be repeated, and the selected nucleic acids mutated and rescreened, whereby a growth factor aptamer is identified. Nucleic acids may be screened to select for molecules that bind to more than target. Binding more than one target can refer to binding more than one simultaneously or competitively. In some embodiments, a binding agent comprises at least one aptamer, wherein a first binding unit binds a first epitope of a polypeptide of the invention and a second binding unit binds a second epitope of the polypeptide.

Binding Agents: Primers, Primer Pairs, Primer Series

Also provided is a primer nucleic acid (or “primer”) comprising a nucleotide sequence which is complementary or substantially complementary to a portion of one of the nucleic acid molecules described herein. By “substantially complementary” as used herein means that the sequence is complementary at all but 3, 2, or 1 nucleotides. It is understood by the ordinarily skilled artisan that primers comprising a nucleotide sequence which is substantially complementary to a portion of one of the nucleic acid molecules described herein can hybridize to the nucleic acid molecule. The inventive primer in exemplary embodiments is modified to comprise a detectable label, such as, for instance, a radioisotope, a fluorophore, and an element particle. The inventive primer is useful in detecting the presence or absence of the fusion gene transcripts, the cDNA thereof, the nucleic acid encoding the fusion gene transcript, and the like. Both qualitative and quantitative analyses may be performed on cells comprising the inventive nucleic acid which encodes the polypeptide. Such analyses include, for example, any type of PCR based assay or hybridization assay, e.g., Southern blot, Northern blot. The sequence of the primer may be designed using online tools such as Primer3 software.

In exemplary aspects, the primer is at least 10 nucleotides in length and is substantially complementary to the sequence of any one of the fusion gene transcripts, the cDNA thereof, and the nucleic acid encoding the fusion gene transcripts described herein. For example, the primer is at least 10 nucleotides in length and is substantially complementary to the sequence of any one of SEQ ID NOs: 1-844, 1001-1844, and 2001-2844. In exemplary aspects, the primer is at least X and no more than Y nucleotides in length, wherein X is 10, 11, 12, 13, 14, or 15 and Y is 20, 21, 22, 23, 24, 25, 26, 27, 28, 29, or 30. In exemplary aspects, the primer is about 10 to about 20 nucleotides in length, about 10 to about 21 nucleotides in length, about 10 to about 22 nucleotides in length, about 10 to about 23 nucleotides in length, about 10 to about 24 nucleotides in length, about 10 to about 25 nucleotides in length, about 10 to about 26 nucleotides in length, about 10 to about 27 nucleotides in length, about 10 to about 28 nucleotides in length, about 10 to about 29 nucleotides in length, or about 10 to about 30 nucleotides in length. In exemplary aspects, the primer is about 11 to about 20 nucleotides in length, about 11 to about 21 nucleotides in length, about 11 to about 22 nucleotides in length, about 11 to about 23 nucleotides in length, about 11 to about 24 nucleotides in length, about 11 to about 25 nucleotides in length, about 11 to about 26 nucleotides in length, about 11 to about 27 nucleotides in length, about 11 to about 28 nucleotides in length, about 11 to about 29 nucleotides in length, or about 11 to about 30 nucleotides in length. In exemplary aspects, the primer is about 12 to about 20 nucleotides in length, about 12 to about 21 nucleotides in length, about 12 to about 22 nucleotides in length, about 12 to about 23 nucleotides in length, about 12 to about 24 nucleotides in length, about 12 to about 25 nucleotides in length, about 12 to about 26 nucleotides in length, about 12 to about 27 nucleotides in length, about 12 to about 28 nucleotides in length, about 12 to about 29 nucleotides in length, or about 12 to about 30 nucleotides in length. In exemplary aspects, the primer is about 13 to about 20 nucleotides in length, about 13 to about 21 nucleotides in length, about 13 to about 22 nucleotides in length, about 13 to about 23 nucleotides in length, about 13 to about 24 nucleotides in length, about 13 to about 25 nucleotides in length, about 13 to about 26 nucleotides in length, about 13 to about 27 nucleotides in length, about 13 to about 28 nucleotides in length, about 13 to about 29 nucleotides in length, or about 13 to about 30 nucleotides in length. In exemplary aspects, the primer is about 14 to about 20 nucleotides in length, about 14 to about 21 nucleotides in length, about 14 to about 22 nucleotides in length, about 14 to about 23 nucleotides in length, about 14 to about 24 nucleotides in length, about 14 to about 25 nucleotides in length, about 14 to about 26 nucleotides in length, about 14 to about 27 nucleotides in length, about 14 to about 28 nucleotides in length, about 14 to about 29 nucleotides in length, or about 14 to about 30 nucleotides in length. In exemplary aspects, the primer is about 15 to about 20 nucleotides in length, about 15 to about 21 nucleotides in length, about 15 to about 22 nucleotides in length, about 15 to about 23 nucleotides in length, about 15 to about 24 nucleotides in length, about 15 to about 25 nucleotides in length, about 15 to about 26 nucleotides in length, about 15 to about 27 nucleotides in length, about 15 to about 28 nucleotides in length, about 15 to about 29 nucleotides in length, or about 15 to about 30 nucleotides in length. In exemplary aspects, the primer is about 15 to about 30 nucleotides in length or about 20 to 30 nucleotides in length or about 25 to 30 nucleotides in length. In exemplary aspects, the primer is about 25 nucleotides in length.

In exemplary aspects, the binding agent is a primer pair comprising a primer as described herein and a second primer. When the binding agent is a primer pair, the primer pair typically comprises a forward primer and a reverse primer. In exemplary aspects, the forward primer comprises a sequence which binds upstream of the targeted sequence while the reverse primer comprises a sequence which binds downstream of the targeted sequence. In exemplary aspects, the targeted sequence is an exon of a gene listed in Column A or Column B of Table 1. In exemplary aspects, the exon is present in the sequence of any one of SEQ ID NOs: 1-844 or 1001-1844. In exemplary aspects, the binding agents of the invention comprises a series of primer pairs, wherein each primer pair of the series binds to a target sequence flanking an exon of each fusion coding sequence listed in the 9^{th} column from the left of Table 1. The series of primer pairs may be used to detect the presence or absence of the fusion transcript or the cDNA thereof.

In alternative embodiments, the targeted sequence comprises the junction of the fusion. The junction of the fusion genes and fusion transcripts of the invention are provided herein by way of providing the location of the junction of each cDNA of the fusion transcript in Table 5. In exemplary aspects, the binding agent comprises a primer pair which targets the junction of the fusion.

In exemplary aspects, the binding agent is a primer pair or a series of primer pairs as described herein, wherein the targeted sequence(s) is/are the cDNA of the fusion transcript.

Kits

The invention further provides kits comprising any one or a combination of the fusion transcripts, polypeptides, nucleic acid molecules, and/or binding agents. The kits are useful in diagnostic methods, research assays, and/or therapeutic methods relating to cancer and tumors. In exemplary embodiments, the kit comprises a binding agent specific for a fusion transcript described herein. In exemplary aspects, the kit comprises a binding agent specific for a nucleic acid encoding the fusion transcript. In exemplary aspects, the kit comprises a binding agent specific for a polypeptide. In exemplary aspects, the binding agents of the kit specifically bind to an epitope of the polypeptide or a target sequence of the fusion transcript or nucleic acid, which encompasses the junction.

In exemplary embodiments, the kit comprises a binding agent that specifically binds to a fusion polypeptide encoded by a fusion transcript encoded by a nucleic acid molecule comprising a structure A-B, wherein structure A is a portion of a gene listed in Column A of Table 1 and structure B is a portion of a gene listed in Column B of Table 1, wherein the gene listed in Column A and the gene listed in Column B are listed in the same row of Table 1, wherein structure B is located immediately 3′ to structure A. In exemplary aspects, the kit comprises a plurality of different binding agents, wherein each binding agent specifically binds to a different fusion gene, fusion transcript or polypeptide listed in one of Tables 1 to 4. In exemplary aspects, the kit comprises at least one binding agent that specifically binds to a fusion transcript encoded by a nucleic acid molecule comprising a structure A-B, wherein structure A is a portion of a gene listed in Column A of Table 1 and structure B is a portion of a gene listed in Column B of Table 1, wherein the gene listed in Column A and the gene listed in Column B are listed in the same row of Table 1 and the row is (a) marked with an asterisk in the 2^{nd} column from the left of Table 1, (b) not marked with a “#” in the 3^{rd} column from the left of Table 1, (c) not marked with a “̂” in the 4^{th} column from the left of Table 1, or (d) a combination thereof, wherein structure B is located immediately 3′ to structure A. In exemplary aspects, the plurality collectively binds to each and every one of the fusion polypeptides listed in Table 1, Table 2, Table 3, or Table 4. In exemplary aspects, the plurality collectively binds to each and every one of the fusion polypeptides listed in Table 1 marked with an asterisk in the 2^{nd} column from the left of Table 1. In exemplary aspects, the plurality collectively binds to each and every one of the fusion polypeptides listed in Table 1 not marked with a “#” in the 3^{rd} column from the left of Table 1. In exemplary aspects, the plurality collectively binds to each and every one of the fusion polypeptides listed in Table 1 not marked with a “̂” in the 4^{th} column from the left of Table 1.

In exemplary aspects, the kit comprises a combination of binding agents wherein the combination specifically binds to at least two different fusion transcripts described herein. In exemplary aspects, the kit comprises a combination of binding agents wherein the combination specifically binds to at least 3, at least 4, at least 5, at least 10, at least 15, at least 20, at least 25, at least 30, at least 40, at least 45, at least 50, at least 55, at least 60, at least 65, at least 70, at least 75, at least 80, at least 85, at least 90, at least 95, at least 100, at least 105, at least 110, at least 115 different fusion transcripts described in Table 1.

In exemplary aspects, the kit comprises a binding agent specific for a fusion transcript (or a polypeptide encoded thereby or a nucleic acid which encodes the fusion transcript) listed in a row Table 1 which is marked with an asterisk.

In exemplary aspects, the binding agents of the kits are primers, primer pairs, or primer pair series, as described herein.

Uses

The invention provides methods of using the fusion transcripts, polypeptides, nucleic acid molecules, and binding agents described herein. As described herein, the fusion transcripts of the invention are recurrent across multiple cancers and thus are useful in detecting a cancer or a tumor in a subject. In exemplary aspects, the fusion transcript occurs at a low frequency in the cancer or tumor.

In exemplary aspects, the binding agents are useful for detecting a cancer or a tumor in a subject. Accordingly, methods of detecting a cancer or a tumor in a subject are provided herein. In exemplary embodiments, the method comprises (i) contacting a binding agent (e.g., an antibody, antigen-binding portion thereof, and the like) that specifically binds to a polypeptide encoded by a fusion transcript of the invention with a sample obtained from the subject and (ii) determining the presence or absence of an immunoconjugate comprising the binding agent and the polypeptide, wherein a cancer or tumor is detected in the subject, when the immunoconjugate is determined as present. Suitable methods of determining the presence or absence of an immunoconjugate are known in the art and include immunoassays (e.g., Western blotting, an enzyme-linked immunosorbent assay (ELISA), a radioimmunoassay (RIA), and immunohistochemical assay.

In exemplary embodiments, the method comprises (i) contacting a binding agent that specifically binds to a fusion transcript of the invention with a sample obtained from the subject, and (ii) determining (a) the structure of the molecule bound to the binding agent or (b) the presence or absence of a double stranded nucleic acid molecule comprising the binding agent and the fusion transcript, when the binding agent binds to a junction region of the fusion transcript comprising a portion of the 3′ end of structure A and a portion of the 5′ end of structure B, wherein a cancer or tumor is detected in the subject, when the structure of the molecule is the structure of the fusion transcript or when the double stranded nucleic acid molecule is determined as present. In exemplary aspects, the binding agent is a primer pair which targets the junction of the fusion gene, the fusion transcript or the cDNA of the fusion transcript. Suitable methods of determining the structure of nucleic acids or the presence or absence of a double stranded nucleic acid molecule are known in the art and include Sanger sequencing, Next-Gen sequencing, eletrophoretic mobility shift assays, quantitative polymerase chain reaction (qPCR), including, but not limited to, real time PCR, Northern blotting and Southern blotting.

In exemplary aspects, the method is based on the detection of cDNA of one or more fusion transcripts. In some aspects, the method comprises producing cDNA with total cellular RNA isolated from cells obtained from the subject as templates. The method may then comprise contacting binding agents that specifically bind to the cDNAs of the fusion transcripts with the cDNAs and detecting binding of the binding agent to the cDNA. Suitable methods of isolating total cellular RNA and producing cDNA therefrom are known in the art and one such method is briefly described herein as Example 7.

In exemplary embodiments, the method comprises (i) generating a population of cDNAs from total RNA isolated from with a sample obtained from the subject, (ii) contacting a binding agent which specifically binds to a nucleic acid molecule comprising the reverse complement (e.g., the reverse complement RNA) sequence of a fusion transcript, with a sample obtained from the subject, and (ii) determining (a) the structure of the molecule bound to the binding agent or (b) the presence or absence of a double stranded nucleic acid molecule comprising the binding agent and the nucleic acid, when the binding agent binds to a sequence which is the reverse complement (e.g., the reverse complement RNA) of a junction region of the fusion transcript comprising a portion of the 3′ end of structure A and a portion of the 5′ end of structure B, wherein a cancer or tumor is detected in the subject, when the structure of the molecule is the structure of the nucleic acid or when the double stranded nucleic acid molecule is determined as present.

In exemplary embodiments, the method of detecting a cancer or a tumor in a subject comprises (i) assaying a sample obtained from the subject for expression of a fusion transcript of the invention, expression of a polypeptide encoded by a fusion transcript of the invention, or presence of a nucleic acid molecule encoding a fusion transcript of the invention, wherein a cancer or tumor is detected in the subject, when the sample is determined as positive for expression of the fusion transcript, expression of the polypeptide or presence of the nucleic acid molecule.

Methods of treating a cancer or a tumor in a subject are also provided herein. In exemplary embodiments, the method comprises (i) assaying a sample obtained from the subject for expression of a fusion transcript of the invention, a polypeptide encoded by a fusion transcript of the invention, or a nucleic acid molecule encoding a fusion transcript of the invention, and (ii) administering to the subject an anti-cancer therapeutic agent in an amount effective for treating a cancer or tumor, when the sample is determined as positive for expression of the fusion transcript, expression of the polypeptide or presence of the nucleic acid molecule.

Methods of determining a subject's need for an anti-cancer therapeutic agent is provided herein. In exemplary embodiments, the method comprises assaying a sample obtained from the subject for expression of a fusion transcript of the invention, a polypeptide encoded by a fusion transcript of the invention, or a nucleic acid molecule encoding a fusion transcript of the invention, wherein the subject needs an anti-cancer therapeutic agent, when the sample is determined as positive for expression of the fusion transcript, expression of the polypeptide or presence of the nucleic acid molecule.

With regard to the methods of treating a cancer or a tumor in a subject and methods of determining a subject's need for an anti-cancer therapeutic agent, the sample may be assayed for expression of the fusion transcript in accordance with any of the methods of detecting a cancer or a tumor in a subject are described herein. Also, with regard to these methods, in exemplary aspects, the anti-cancer therapeutic is one described herein under “Therapeutic Agents.”

Suitable methods of assaying samples for fusion transcripts, polypeptides encoded thereby, or for nucleic acids encoding the fusion transcripts are known in the art and include, but not limited to, Sanger sequencing, Next-Gen sequencing, eletrophoretic mobility shift assays, quantitative polymerase chain reaction (qPCR), real time PCR, Northern blotting, Southern blotting, immunoassays (e.g., Western blotting, an enzyme-linked immunosorbent assay (ELISA), a radioimmunoassay (RIA), and immunohistochemical assays).

Therapeutic Agents

Provided herein are therapeutic agents which target the fusion transcripts or polypeptides of the invention. In exemplary embodiments, the therapeutic agent an antibody or antigen binding fragment or the like which binds to the antigen (e.g., the polypeptide encoded by the fusion transcript) and which neutralizes the biological activity of the polypeptide.

In exemplary embodiments, the therapeutic agent is an antisense nucleic acid molecule which binds to the fusion transcript and prevents the production of the resulting polypeptide. In exemplary embodiments, the therapeutic agent is an antisense nucleic acid molecule which binds to a nucleic acid which encodes the fusion transcript and which prevents the production of the fusion transcript. The antisense molecule in exemplary aspects is about 5, about 10, about 15, about 20, about 25, about 30, about 35, about 40, about 45 or about 50 nucleotides in length. In exemplary aspects, the antisense molecule is about X to about Y nucleotides in length, wherein X is 10, 11, 12, 13, 14, or 15 and Y is 20, 21, 22, 23, 24, 25, 26, 27, 28, 29, or 30. In exemplary aspects, the antisense molecule is about 10 to about 20 nucleotides in length, about 10 to about 21 nucleotides in length, about 10 to about 22 nucleotides in length, about 10 to about 23 nucleotides in length, about 10 to about 24 nucleotides in length, about 10 to about 25 nucleotides in length, about 10 to about 26 nucleotides in length, about 10 to about 27 nucleotides in length, about 10 to about 28 nucleotides in length, about 10 to about 29 nucleotides in length, or about 10 to about 30 nucleotides in length. In exemplary aspects, the antisense molecule is about 11 to about 20 nucleotides in length, about 11 to about 21 nucleotides in length, about 11 to about 22 nucleotides in length, about 11 to about 23 nucleotides in length, about 11 to about 24 nucleotides in length, about 11 to about 25 nucleotides in length, about 11 to about 26 nucleotides in length, about 11 to about 27 nucleotides in length, about 11 to about 28 nucleotides in length, about 11 to about 29 nucleotides in length, or about 11 to about 30 nucleotides in length. In exemplary aspects, the antisense molecule is about 12 to about 20 nucleotides in length, about 12 to about 21 nucleotides in length, about 12 to about 22 nucleotides in length, about 12 to about 23 nucleotides in length, about 12 to about 24 nucleotides in length, about 12 to about 25 nucleotides in length, about 12 to about 26 nucleotides in length, about 12 to about 27 nucleotides in length, about 12 to about 28 nucleotides in length, about 12 to about 29 nucleotides in length, or about 12 to about 30 nucleotides in length. In exemplary aspects, the antisense molecule is about 13 to about 20 nucleotides in length, about 13 to about 21 nucleotides in length, about 13 to about 22 nucleotides in length, about 13 to about 23 nucleotides in length, about 13 to about 24 nucleotides in length, about 13 to about 25 nucleotides in length, about 13 to about 26 nucleotides in length, about 13 to about 27 nucleotides in length, about 13 to about 28 nucleotides in length, about 13 to about 29 nucleotides in length, or about 13 to about 30 nucleotides in length. In exemplary aspects, the antisense molecule is about 14 to about 20 nucleotides in length, about 14 to about 21 nucleotides in length, about 14 to about 22 nucleotides in length, about 14 to about 23 nucleotides in length, about 14 to about 24 nucleotides in length, about 14 to about 25 nucleotides in length, about 14 to about 26 nucleotides in length, about 14 to about 27 nucleotides in length, about 14 to about 28 nucleotides in length, about 14 to about 29 nucleotides in length, or about 14 to about 30 nucleotides in length. In exemplary aspects, the antisense molecule is about 15 to about 20 nucleotides in length, about 15 to about 21 nucleotides in length, about 15 to about 22 nucleotides in length, about 15 to about 23 nucleotides in length, about 15 to about 24 nucleotides in length, about 15 to about 25 nucleotides in length, about 15 to about 26 nucleotides in length, about 15 to about 27 nucleotides in length, about 15 to about 28 nucleotides in length, about 15 to about 29 nucleotides in length, or about 15 to about 30 nucleotides in length. In exemplary aspects, the antisense molecule is about 15 to about 30 nucleotides in length or about 20 to 30 nucleotides in length or about 25 to 30 nucleotides in length. In exemplary aspects, the antisense molecule is about 25 nucleotides in length.

In exemplary aspects, the antisense molecule is an antisense oligonucleotide or antisense nucleic acid analog which is complementary to at least a portion of a sequence of any one of SEQ ID NOs: 1-844, 1001-1844, and 2001-2844. The antisense molecule in some aspects is complementary to at least 15 contiguous bases of said sequence. The antisense molecule in some aspects is complementary to at least 20 contiguous bases of said sequence, at least 25 contiguous bases of the sequence. In exemplary aspects, the antisense molecule is an antisense oligonucleotide or antisense nucleic acid analog comprising at least 15 contiguous bases, which are complementary sequences to a portion of a sequence of any one of SEQ ID NOs: 1-844, 1001-1844, and 2001-2844. In exemplary aspects, the antisense molecule is an antisense oligonucleotide or antisense nucleic acid analog comprising at least 15 contiguous bases that differs by not more than 3 bases from a portion of 15 contiguous bases of said SEQ ID NOs.

The antisense molecule can be one which mediates RNA interference (RNAi). As known by one of ordinary skill in the art, RNAi is a ubiquitous mechanism of gene regulation in plants and animals in which target mRNAs are degraded in a sequence-specific manner (Sharp, Genes Dev., 15, 485-490 (2001); Hutvagner et al., Curr. Opin. Genet. Dev., 12, 225-232 (2002); Fire et al., Nature, 391, 806-811 (1998); Zamore et al., Cell, 101, 25-33 (2000)). The natural RNA degradation process is initiated by the dsRNA-specific endonuclease Dicer, which promotes cleavage of long dsRNA precursors into double-stranded fragments between 21 and 25 nucleotides long, termed small interfering RNA (siRNA; also known as short interfering RNA) (Zamore, et al., Cell. 101, 25-33 (2000); Elbashir et al., Genes Dev., 15, 188-200 (2001); Hammond et al., Nature, 404, 293-296 (2000); Bernstein et al., Nature, 409, 363-366 (2001)). siRNAs are incorporated into a large protein complex that recognizes and cleaves target mRNAs (Nykanen et al., Cell, 107, 309-321 (2001). It has been reported that introduction of dsRNA into mammalian cells does not result in efficient Dicer-mediated generation of siRNA and therefore does not induce RNAi (Caplen et al., Gene 252, 95-105 (2000); Ui-Tei et al., FEBS Lett, 479, 79-82 (2000)). The requirement for Dicer in maturation of siRNAs in cells can be bypassed by introducing synthetic 21-nucleotide siRNA duplexes, which inhibit expression of transfected and endogenous genes in a variety of mammalian cells (Elbashir et al., Nature, 411: 494-498 (2001)).

In this regard, the antisense molecule of the invention in some aspects mediates RNAi and in some aspects is a siRNA molecule specific for inhibiting the expression of the fusion transcript and/or the polypeptide encoded thereby. The term “siRNA” as used herein refers to an RNA (or RNA analog) comprising from about 10 to about 50 nucleotides (or nucleotide analogs) which is capable of directing or mediating RNAi. In exemplary embodiments, an siRNA molecule comprises about 15 to about 30 nucleotides (or nucleotide analogs) or about 20 to about 25 nucleotides (or nucleotide analogs), e.g., 21-23 nucleotides (or nucleotide analogs). The siRNA can be double or single stranded, preferably double-stranded.

In alternative aspects, the antisense molecule is alternatively a short hairpin RNA (shRNA) molecule specific for inhibiting the expression of the fusion transcript and/or the polypeptide encoded thereby. The term “shRNA” as used herein refers to a molecule of about 20 or more base pairs in which a single-standed RNA partially contains a palindromic base sequence and forms a double-strand structure therein (i.e., a hairpin structure). An shRNA can be an siRNA (or siRNA analog) which is folded into a hairpin structure. shRNAs typically comprise about 45 to about 60 nucleotides, including the approximately 21 nucleotide antisense and sense portions of the hairpin, optional overhangs on the non-loop side of about 2 to about 6 nucleotides long, and the loop portion that can be, e.g., about 3 to 10 nucleotides long. The shRNA can be chemically synthesized. Alternatively, the shRNA can be produced by linking sense and antisense strands of a DNA sequence in reverse directions and synthesizing RNA in vitro with T7 RNA polymerase using the DNA as a template.

Though not wishing to be bound by any theory or mechanism it is believed that after shRNA is introduced into a cell, the shRNA is degraded into a length of about 20 bases or more (e.g., representatively 21, 22, 23 bases), and causes RNAi, leading to an inhibitory effect. Thus, shRNA elicits RNAi and therefore can be used as an effective component of the disclosure. shRNA may preferably have a 3′-protruding end. The length of the double-stranded portion is not particularly limited, but is preferably about 10 or more nucleotides, and more preferably about 20 or more nucleotides. Here, the 3′-protruding end may be preferably DNA, more preferably DNA of at least 2 nucleotides in length, and even more preferably DNA of 2-4 nucleotides in length.

In exemplary aspects, the antisense molecule is a microRNA (miRNA). As used herein the term “microRNA” refers to a small (e.g., 15-22 nucleotides), non-coding RNA molecule which base pairs with mRNA molecules to silence gene expression via translational repression or target degradation. microRNA and the therapeutic potential thereof are described in the art. See, e.g., Mulligan, MicroRNA: Expression, Detection, and Therapeutic Strategies, Nova Science Publishers, Inc., Hauppauge, N.Y., 2011; Bader and Lammers, “The Therapeutic Potential of microRNAs” Innovations in Pharmaceutical Technology, pages 52-55 (March 2011)

In exemplary aspects, the antisense molecule is an antisense oligonucleotide comprising DNA or RNA or both DNA and RNA. In exemplary aspects, the antisense oligonucleotide comprises naturally-occurring nucleotides and/or naturally-occurring internucleotide linkages. The antisense oligonucleotide in some aspects is single-stranded and in other aspects is double-stranded. In exemplary aspects, the antisense oligonucleotide is synthesized and in other aspects is obtained (e.g., isolated and/or purified) from natural sources. In exemplary aspects, the antisense molecule is a phosphodiester oligonucleotide.

In alternative aspects, the antisense molecule is an antisense nucleic acid analog, e.g., comprising non-naturally-occurring nucleotides and/or non-naturally-occurring internucleotide linkages (e.g., phosphoroamidate linkages, phosphorothioate linkages). In exemplary aspects, the antisense nucleic acid analog comprises one or more modified nucleotides, including, but not limited to, 5-fluorouracil, 5-bromouracil, 5-chlorouracil, 5-iodouracil, hypoxanthine, xanthine, 4-acetylcytosine, 5-(carboxyhydroxymethyl) uracil, 5-carboxymethylaminomethyl-2-thiouridine, 5-carboxymethylaminomethyluracil, dihydrouracil, beta-D-galactosylqueuosine, inosine, N⁶-isopentenyladenine, 1-methylguanine, 1-methylinosine, 2,2-dimethylguanine, 2-methyladenine, 2-methylguanine, 3-methylcytosine, 5-methylcytosine, N-substituted adenine, 7-methylguanine, 5-methylammomethyluracil, 5-methoxyaminomethyl-2-thiouracil, beta-D-mannosylqueuosine, 5′-methoxycarboxymethyluracil, 5-methoxyuracil, 2-methylthio-N⁶-isopentenyladenine, uracil-5-oxyacetic acid (v), wybutoxosine, pseudouracil, queuosine, 2-thiocytosine, 5-methyl-2-thiouracil, 2-thiouracil, 4-thiouracil, 5-methyluracil, uracil-5-oxyacetic acid methylester, 3-(3-amino-3-N-2-carboxypropyl) uracil, and 2,6-diaminopurine.

In exemplary aspects, the antisense nucleic acid analog comprises non-naturally-occurring nucleotides which differ from naturally occurring nucleotides by comprising a ring structure other than ribose or 2-deoxyribose. In exemplary aspects, the antisense nucleic acid comprises non-naturally-occurring nucleotides which differ from naturally occurring nucleotides by comprising a chemical group in place of the phosphate group.

In exemplary aspects, the antisense nucleic acid analog comprises or is a methylphosphonate oligonucleotide, which are noncharged oligomers in which a non-bridging oxygen atom is replaced by a methyl group at each phosphorous in the oligonucleotide chain. In exemplary aspects, the antisense nucleic acid analog comprises or is a phosphorothioate, wherein at least one of the non-bridging oxygen atom is replaced by a sulfur at each phosphorous in the oligonucleotide chain.

In exemplary aspects, the antisense nucleic acid analog is an analog comprising a replacement of the hydrogen at the 2′-position of ribose with an O-alkyl group, e.g., methyl. In exemplary aspects, the antisense nucleic acid analog comprises a modified ribonucleotide wherein the 2′ hydroxyl of ribose is modified to methoxy (OMe) or methoxy-ethyl (MOE) group. In exemplary aspects, the antisense nucleic acid analog comprises a modified ribonucleotide wherein the 2′ hydroxyl of ribose is allyl, amino, azido, halo, thio, O-allyl, O—C₁-C₁₀ alkyl, O—C₁-C₁₀ substituted alkyl, O—C₁-C₁₀ alkoxy, O—C₁-C₁₀ substituted alkoxy, OCF₃, O(CH₂)₂SCH₃, O(CH₂)₂—O—N(R¹)(R²), or O(CH₂)—C(═O)—N(R¹)(R²), wherein each of R¹ and R² is independently selected from the group consisting of H, an amino protecting group or substituted or unsubstituted C₁-C₁₀ alkyl. In exemplary aspects, the antisense nucleic acid analog comprises a modified ribonucleotide wherein the 2′ hydroxyl of ribose is 2′F, SH, CN, OCN, CF₃, O-alkyl, S-Alkyl, N(R¹)alkyl, O-alkenyl, S-alkenyl, or N(R¹)-alkenyl, O-alkynyl, S-alkynyl, N(R¹)-alkynyl, O-alkylenyl, O-Alkyl, alknyyl, alkaryl, aralkyl, O-alkaryl, or O-aralkyl.

In exemplary aspects, the antisense nucleic acid analog comprises a substituted ring. In exemplary aspects, the antisense nucleic acid analog is or comprises a hexitol nucleic acid. In exemplary aspects, the antisense nucleic acid analog is or comprises a nucleotide with a bicyclic or tricyclic sugar moiety. In exemplary aspects, the bicyclic sugar moiety comprises a bridge between the 4′ and 2′ furanose ring atoms. Examplary moieties include, but are not limited to: —[C(Rₐ)(R_{b})]ₙ—, —[C(Rₐ)(R_{b})]ₙ-0-, —C(RₐR_{b})—N(R)-0- or, —C(RₐR_{b})-0-N(R)—; 4′-CH₂-2′, 4′-(CH₂)₂-2′, 4′-(CH₂)₃-2′, 4′-(CH₂)-0-2′ (LNA); 4′-(CH₂)—S-2′; 4′-(CH₂)₂-0-2′ (ENA); 4′-CH(CH₃)-0-2′ (cEt) and 4′-CH(CH₂OCH₃)-0-2′, 4′-C(CH₃)(CH₃)-0-2′, 4′-CH₂—N(OCH₃)-2′, 4′-CH₂-0-N(CH₃)-2′ 4′-CH₂-0-N(R)-2′, and 4′-CH₂—N(R)-0-2′-, wherein each R is, independently, H, a protecting group, or C₁C₁₂ alkyl; 4′-CH₂—N(R)-0-2′, wherein R is H, C1-C12 alkyl, or a protecting group, 4′-CH₂—C(H)(CH₃)-2′, 4′-CH₂—C(═CH₂)-2′. Such antisense nucleic acid analogs are known in the art. See, e.g., International Application Publication No. WO 2008/154401, U.S. Pat. No. 7,399,845, International Application Publication No. WO2009/006478, International Application Publication No. WO2008/150729, U.S. Application Publication No. US2004/0171570, U.S. Pat. No. 7,427,672, and Chattopadhyaya, et al, J. Org. Chem., 2009, 74, 118-134). In exemplary aspects, the antisense nucleic acid analog comprises a nucleoside comprising a bicyclic sugar moiety, or a bicyclic nucleoside (BNA). In exemplary aspects, the antisense nucleic acid analog comprises a BNA selected from the group consisting of: α-L-Methyleneoxy (4′-CH₂-0-2′) BNA, Aminooxy (4′-CH₂-0-N(R)-2′) BNA, β-D-Methyleneoxy (4′-CH₂-0-2′) BNA, Ethyleneoxy (4′-(CH₂)₂-0-2′) BNA, methylene-amino (4′-CH2-N(R)-2′) BNA, methyl carbocyclic (4′-CH₂—CH(CH₃)-2′) BNA, Methyl(methyleneoxy) (4′-CH(CH₃)-0-2′) BNA (also known as constrained ethyl or cEt), methylene-thio (4′-CH₂—S-2′) BNA, Oxyamino (4′-CH₂—N(R)-0-2′) BNA, and propylene carbocyclic (4′-(CH₂)₃-2′) BNA. Such BNAs are described in the art. See, e.g., International Patent Publication No. WO 2014/071078.

In exemplary aspects, the antisense nucleic acid analog comprises a modified backbone. In exemplary aspects, the antisense nucleic acid analog is or comprises a peptide nucleic acid (PNA) containing an uncharged flexible polyamide backbone comprising repeating N-(2-aminoethyl)glycine units to which the nucleobases are attached via methylene carbonyl linkers. In exemplary aspects, the antisense nucleic acid analog comprises a backbone substitution. In exemplary aspects, the antisense nucleic acid analog is or comprises an N3′→P5′ phosphoramidate, which results from the replacement of the oxygen at the 3′ position on ribose by an amine group. Such nucleic acid analogs are further described in Dias and Stein, Molec Cancer Ther 1: 347-355 (2002). In exemplary aspects, the antisense nucleic acid analog comprises a nucleotide comprising a conformational lock. In exemplary aspects, the antisense nucleic acid analog is or comprises a locked nucleic acid.

In exemplary aspects, the antisense nucleic acid analog comprises a 6-membered morpholine ring, in place of the ribose or 2-deoxyribose ring found in RNA or DNA. In exemplary aspects, the antisense nucleic acid analog comprises non-ionic phophorodiamidate intersubunit linkages in place of anionic phophodiester linkages found in RNA and DNA. In exemplary aspects, the nucleic acid analog comprises nucleobases (e.g., adenine (A), cytosine (C), guanine (G), thymine, thymine (T), uracil (U)) found in RNA and DNA. In exemplary aspects, the IRES inhibitor is a Morpholino oligomer comprising a polymer of subunits, each subunit of which comprises a 6-membered morpholine ring and a nucleobase (e.g., A, C, G, T, U), wherein the units are linked via non-ionic phophorodiamidate intersubunit linkages. For purposes herein, when referring to the sequence of a Morpholino oligomer, the conventional single-letter nucleobase codes (e.g., A, C, G, T, U) are used to refer to the nucleobase attached to the morpholine ring.

Biological Samples

With regard to the methods disclosed herein, in some embodiments, the sample comprises a bodily fluid, including, but not limited to, blood, plasma, serum, lymph, breast milk, saliva, mucous, semen, vaginal secretions, cellular extracts, inflammatory fluids, cerebrospinal fluid, feces, vitreous humor, or urine obtained from the subject. In some aspects, the sample is a composite panel of at least two of the foregoing samples. In some aspects, the sample is a composite panel of at least two of a blood sample, a plasma sample, a serum sample, and a urine sample. In exemplary aspects, the sample comprises blood or a fraction thereof (e.g., plasma, serum, fraction obtained via leukopheresis). In exemplary aspects, the biological sample comprises cancer cells or tumor cells. In exemplary aspects, the biological sample is a biopsied sample.

Subjects

With regard to the methods disclosed herein, the subject in exemplary aspects is a mammal, including, but not limited to, mammals of the order Rodentia, such as mice and hamsters, and mammals of the order Logomorpha, such as rabbits, mammals from the order Carnivora, including Felines (cats) and Canines (dogs), mammals from the order Artiodactyla, including Bovines (cows) and Swines (pigs) or of the order Perssodactyla, including Equines (horses). In some aspects, the mammals are of the order Primates, Ceboids, or Simoids (monkeys) or of the order Anthropoids (humans and apes). In some aspects, the mammal is a human.

Cancer and Tumors

The cancer in exemplary aspects is one selected from the group consisting of acute lymphocytic cancer, acute myeloid leukemia, alveolar rhabdomyosarcoma, bone cancer, brain cancer, breast cancer, cancer of the anus, anal canal, or anorectum, cancer of the eye, cancer of the intrahepatic bile duct, cancer of the joints, cancer of the neck, gallbladder, or pleura, cancer of the nose, nasal cavity, or middle ear, cancer of the oral cavity, cancer of the vulva, chronic lymphocytic leukemia, chronic myeloid cancer, colon cancer, esophageal cancer, cervical cancer, gastrointestinal carcinoid tumor, Hodgkin lymphoma, hypopharynx cancer, kidney cancer, larynx cancer, liver cancer, lung cancer, malignant mesothelioma, melanoma, multiple myeloma, nasopharynx cancer, non-Hodgkin lymphoma, ovarian cancer, pancreatic cancer, peritoneum, omentum, and mesentery cancer, pharynx cancer, prostate cancer, rectal cancer, renal cancer (e.g., renal cell carcinoma (RCC)), small intestine cancer, soft tissue cancer, stomach cancer, testicular cancer, thyroid cancer, ureter cancer, and urinary bladder cancer. In particular aspects, the cancer is selected from the group consisting of: head and neck, ovarian, cervical, bladder and oesophageal cancers, pancreatic, gastrointestinal cancer, gastric, breast, endometrial and colorectal cancers, hepatocellular carcinoma, glioblastoma, bladder, lung cancer, e.g., non-small cell lung cancer (NSCLC), bronchioloalveolar carcinoma.

As used herein, the term “tumor” refers to any tumor cell, including but not limited to a tumor cell of one of the following: Tumor Type Data Status Acute Myeloid Leukemia (AML), Breast cancer (BRCA), Chromophobe renal cell carcinoma (KICH), Clear cell kidney carcinoma (KIRC), Colon and rectal adenocarcinoma (COAD, READ), Cutaneous melanoma (SKCM), Glioblastoma multiforme (GBM), Head and neck squamous cell carcinoma (HNSC), Lower Grade Glioma (LGG), Lung adenocarcinoma (LUAD), Lung squamous cell carcinoma (LUSC), Ovarian serous cystadenocarcinoma (OV), Papillary thyroid carcinoma (THCA), Stomach adenocarcinoma (STAD), Prostate adenocarcinoma (PRAD), Uterine corpus endometrial carcinoma (UCEC), Urothelial bladder cancer (BLCA), Papillary kidney carcinoma (KIRP), Liver hepatocellular carcinoma (LIHC), Cervical cancer (CESC), Uterine carcinosarcoma (UCS), Adrenocortical carcinoma (ACC), Esophageal cancer (ESCA), Pheochromocytoma & Paraganglioma (PCPG), Pancreatic ductal adenocarcinoma (PAAD), Diffuse large B-cell lymphoma (DLBC), Cholangiocarcinoma (CHOL), Mesothelioma (MESO), Sarcoma (SARC), Testicular germ cell cancer (TGCT), Uveal melanoma (UVM).

The following examples serve only to illustrate the invention or provide background information relating to the invention. The following examples are not intended to limit the scope of the invention in any way.

### EXAMPLES

### Example 1

To fully characterize the landscape of gene fusions across multiple cancers, a novel algorithm, MOJO (Minimum Overlap Junction Optimizer) was developed. MOJO uses paired-end transcriptome sequencing data to detect fusions with high sensitivity and specificity. Extensive performance evaluations of MOJO in comparison with eight previously published methods was performed using a compendium of eighteen previously published cell line transcriptomes. MOJO demonstrated the highest sensitivity and specificity among the methods compared.

Using MOJO, fusion discovery on 9,704 tumors across 33 cancer types in the Cancer Genome Atlas (TCGA) was performed. Several heuristic filters were further developed and applied to exclude spurious recurrent fusions that could manifest in such large pan-cancer analysis. A subset of fusions detected in our screen could be due to germline gene fusions that are the result of copy number variation in human populations (Chase et al., Haematologica 95(1): 20-26 (2010)). To account for this possibility, 3,600 cell line and tissue transcriptomes from healthy individuals were analyzed and all fusions that were detected at <5× enrichment in primary tumors were excluded. These filtering criteria were extremely stringent in enriching for strictly somatic events. For example, we detected previously well characterized oncogenic fusion BCR-ABL1 in 7 normal tissues and is detected at similar frequency in the tumor transcriptomes. It was proposed that fusions detected in normal tissues are sub-clonal (i.e, fusion is generated in a very small sub-population of cells and selected because it confers a selective advantage). In all, 22% of the fusion genes were excluded after incorporating the normal data. Table 3 lists those fusions which remained after the filtering criteria was applied.

22,289 high confidence somatic fusion calls comprising 16,531 distinct fusion genes were nominated. Across 33 cancer types, we identified 124 highly recurrent (≥5 tumors across cancers) protein coding fusion genes with breakpoints clustered in at least one of the genes involved in the fusion (low entropy), suggesting that these are not consequences of focal SCNAs. 26 (21%) of these are previously known, and, we found that 24 out of 33 cancer types studied here have at least one tumor with a known fusion. Interestingly, we found that 60% (14/22) of these known recurrent fusions in tumors of epithelial origin were detected in multiple cancer types. For example, we found targetable FGFR3::TACC3 fusion in twelve cancer types, seven more than previously reported. We found an ESR1::CCDC170 fusion in uterine corpus endometrial carcinoma, uterine carcinosarcoma and ovarian, in addition to the previously reported, breast cancer. All four cancers are estrogen driven suggesting a shared mechanism. Wnt pathway activating and potentially actionable PTPRK::RSPO3 is detected in esophageal and gastric tissue tumors, in addition to the colon and rectal cancers in which this fusion was first discovered.

Consistent with the patterns of previously known recurrent fusions across cancers, we found that 91.8% (90) of novel recurrent fusions were detected in multiple cancer types, and, therefore, highlighting the importance of screening all cancer diagnoses with a comprehensive panel of therapeutically responsive fusions. Among these, we identified 59 highly recurrent fusions that are detected in multiple cancers and are hypothesized to have a functional role (Table 1 fusions marked with * and not marked with #). These highly recurrent fusions present compelling hypotheses to their role in tumor progression.

For example, the fusion gene BMPR1B-PDLIM5, seen in 28 tumors of Breast, Prostate and Ovarian cancers (all hormone driven), generates a novel truncated PDLIM5 gene that loses a phosphorylation site and retains the C-terminus LIM domains. A previous study has shown that the phosphorylation site is essential to inhibit migration (Yan et al., Nat Commun 6:6137 (2015)). In an another example, we found 59 tumors in all of TCGA that have a fusion gene that results in BCAR4 fused to the 3′-end of the fusion. First identified in tamoxifen resistance screen, BCAR4 overexpression has been shown to induce anchorage independent growth in estrogen dependent ZR-75-1 breast cancer cell line (Godinho et al., Br J Cancer 103(8): 2384-1291 (2010)). We hypothesized that a fusion event is common mechanism with which the BCAR4 is over-expressed in cancers. In a third example, we discovered a novel fusion gene that is the result of a tandem duplication event that fuses LIM domain containing 7 (LMO7) and ubiquitin carboxyl-terminal esterase L3 (UCHL3). We found this fusion in 65 tumors across 16 cancers (6 in breast) with the most predominant isoform fusing the first exon of LMO7 to the second exon of UCHL3. The resulting protein is contains the complete enzymatic domain of UCHL3. Higher expression of UCHL3 has been previously reported to be associated with invasive breast cancer (Miyoshi et al., Cancer Sci 97(6): 523-529 (2006)). In a fourth example, we discovered a novel fusion that is the result of a translocation event and fuses the thymidylate synthetase gene (TYMS) on 18p11 to septin-9 (SEPT9) on 17q25. 11 tumors in three different cancer types are predicted to have this fusion. Interestingly, SEPT9 has been previously reported as a fusion partner of MLL in therapy related acute myeloid leukemia (Osaka et al., PNAS 96(11): 6428-6433 (1999)). SEPT9 overexpression has been shown to promote mesenchymal-like migration of renal cells and correspondingly, SEPT9 knockdown decreased migration (Dolat et al., J Cell Biol 207: 225-235 (2014); Estey et al., J Cell Biol 191: 741-749 (2010)).

Additional novel and highly recurrent fusions are functionally evaluated and biologically characterized as described herein.

### Example 2

This example describes the generation of stable cell lines expressing the fusions in MCF10A benign breast epithelial cells.

To functionally evaluate each fusion gene transcript, the fusion genes were synthesized and stable cell lines with the fusion gene integrated in the genome were generated. In one example, MCF10A, a breast epithelial cell line, was chosen as the genetic background in which the function of select fusions were analyzed. MCF10A is a non-malignant cell line that has been previously used to evaluate the effects of oncogenic mutations both in-vitro and in-vivo (Soule et al., Cancer Res 50(18): 60756086 (1990)). For the first phase of experiments, 14 fusion genes were selected, mainly based on their recurrence level as well as the ability to synthesize the construct. We synthesized the fusion genes and generated MCF10A cell lines stably expressing these fusion genes.

### Example 3

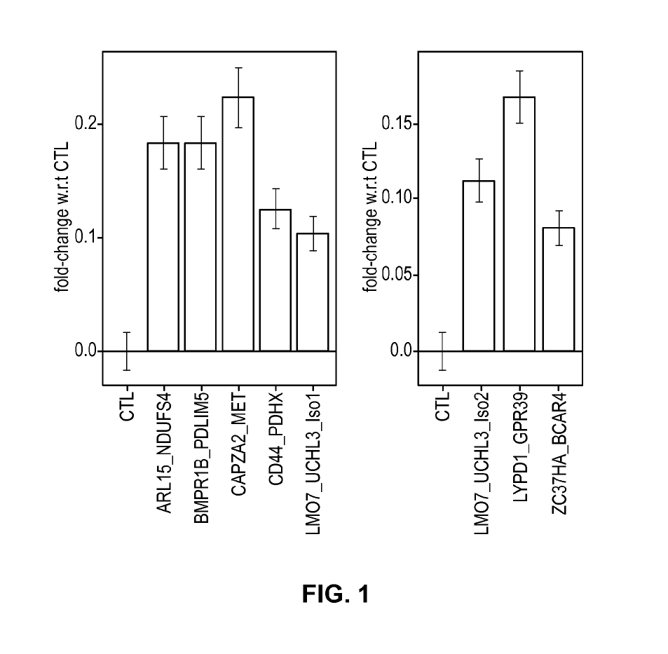

Using the stable cell lines described in Example 2, the role in proliferation of seven fusion gene transcripts was analyzed. In-vitro proliferation assays as essentially described in White et al., Nature 471 (7339): 518-522 (2011)) were performed in triplicate in 384-well plates. A total of seven stable cell lines, each expressing a different fusion gene transcript, was used in these assays. The stable cell lines expressed one of ARL15_NDUFS4; BMPR1B_PDLIM5; CAPZA2_MET; CD44_PDHX; LMO7_UCHL3. Each cell line was plated in 16 wells of a plate at a density of 400 cells/well. Proliferation rates were measured on Day 4 using the CellTiterGlo® assay kit from Promega (Madison, Wis.). Proliferation measurements were normalized for with- and across-plate batch effects and compared to a control cell line to determine change in proliferation. All seven cell lines showed statistically significant increase in proliferation (FIG. 1).

### Example 4

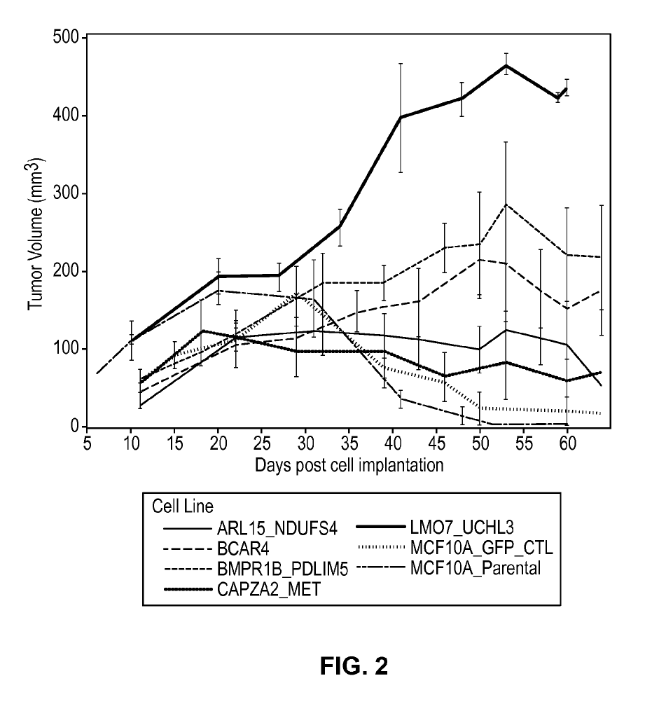

Five of the stable cell lines that demonstrated an in-vitro increase in proliferation were selected for in-vivo assay for tumor growth in mice. These were stable cells lines expressing ARL15_NDUFS4; BMPR1B_PDLIM5; CAPZA2_MET; CD44_PDHX; LMO7_UCHL3. Xenograft assays were performed as described in Moyano et al., J Clin Invest 116(1): 261-270 (2006). To determine if over expression of the fusions is itself sufficient to induce tumor growth in mice, mouse mammary fat pads were inoculated with MCF10A fusion-positive cell lines in the presence of Matrigel. The five fusion cell lines along with the GFP-only control and parental MCF10A cell line were tested. Three of the fusion cell lines, BMPR1B-PDLIM5, ZC3H7A-BCAR4 and LMO7-UCHL3 showed palpable tumors at week 5 with increasing tumor volume till week 9 and neither the GFP-only control nor the parental MCF10A control showed tumor growth (FIG. 2). For two fusion cell lines, ARL15-NDUFS4 and CAPZA2-MET, an in vivo phenotype was not observed. It is thought that the benign MCF10A genetic background may not be sufficient to induce tumorigenesis without supporting mutations. For example, unlike the three fusions that showed in-vivo tumor growths, these two fusions were only detected in one tumor sample each, in the breast cancer cohort. ARL15-NDUFS4 is detected at high frequency in 26 (5%) of lung squamous cell carcinoma and CAPZA2-MET in 4 (1%) lung adenocarcinoma samples suggesting that these fusions when expressed in tissue types other than that of MCF10A may exhibit a tumorigenic phenotypes. In addition, for a vast majority of these fusions, co-occurring mutations in a specific pathway that may occur, in conjunction with the fusion, to confer proliferation advantage to cells. Therefore, the stable cell lines will be tested and evaluated in other cell lines, including malignant ones.

### Example 5

Fusion transcripts BMPR1B-PDLIM5, ZC3H7A-BCAR4 or LMO7-UCHL3 are evaluated in additional genetic backgrounds: MCF7 (estrogen-receptor positive, invasive ductal breast carcinoma), MDA-MB-231 (triple negative breast cancer) and NIH3T3 (mouse embryonic fibroblast) cell lines. The fusion transcripts are stably expressed in these cells lines and then evaluated for a hormone dependence. The stable cell lines are used in in-vitro proliferation assays and in-vivo proliferation assays. In these assays, tumor progression in mice is monitored and siRNAs targeting the fusion junction to evaluate the tumor response to repression of fusion gene expression are administered to the mice. Tumor progression in the mice following siRNA administration is monitored.

Stable cells lines are made for each and every one of the 58 novel recurrent fusions reported here. The stable cell lines are then used in the proliferation and tumor growth assays described in Examples 3 and 4.

For fusions that do not show phenotype in the MCF10A background, the fusion transcript is expressed in the genetic background (tumor tissue type) where it is deemed as expressed at high frequency. For example, ARL15-NDUFS4, which is detected at high frequency in lung squamous cell carcinoma and which failed to show a phenotype in MCF10A, is expressed in SW900, a squamous cell carcinoma cell line and assay for phenotype. In this manner, a rigorous case-by-case approach is taken to identify the appropriate genetic background in which to evaluate the fusion. In addition, for fusions with co-occurring mutations, mutations are introduced in the transfected cell lines using CRISPR/Cas9 system and assayed for tumorigenic phenotypes.

### Example 6

To evaluate the fusion gene transcripts for cellular migration and invasion phenotypes, in vitro experiments are carried out as previously described (Ma et al., Nature 449(7163): 682-688 (2007)). Fusion gene transcripts produced in late stage tumors might confer a migratory or invasive phenotype that accelerate tumor progression. Using a Boyden chamber transwell migration and invasion assay, cell motility and their ability to migrate through the extra-cellular matrix or basement membrane extract is quantified.

### Example 7

The presence or absence of fusion gene transcripts is assayed in a biological sample obtained from a subject following the methods described in van Dongen et al., Leukemia 13(12): 1901-1928 (1999). Briefly, total cellular RNA is isolated from a tissue sample obtained from a subject using an RNeasy® purification kit (Qiagen, Venlo, Limburg). Using the isolated RNA as a template, cDNA is synthesized using the SuperScript® III Reverse Transcriptase kit (Life Technologies, Carlsbad, Calif.). A priori primers specific for the recurrent fusions reported here are designed using Primer3, a free online tool to design and analyze primers for PCR and real time PCR experiments. Primers are synthesized and used to assay for the presence or absence of each fusion transcript using PCR. Gels are run to identify and extract the PCR product. Each identified band is sequenced using Sanger sequencing. The sequence obtained is used to establish the presence or absence of the fusion. Further details for carrying this assay out are published in van Dongen et al., Leukemia 13(12): 1901-28 (1999). The output of the PCR reactions are also assessed for the presence of the fusion transcript by pooling the PCR products and sequencing them using next-generation sequencing.

A strictly high-throughput sequencing based assay is developed to detect the fusion transcripts. The primary component of this assay is the biotin-tagged capture probe sequences designed to capture the exons comprising the fusion transcripts. More specifically, each exon predicted to be involved in the fusion transcripts described here are targeted by the capture probe sequence. Using these probes, the cDNA sequences containing the targeted exons are isolated and subsequently sequenced using next-generation sequencing. A computational method, similar to MOJO, is used to identify fusion junctions from the sequencing output. An outline of our approach is described in Ueno et al., Cancer Sci 103-1: 131-135 (2012).

TABLE 5

| | | Location of | Location of |
|---|---|---|---|
| | | Junction is | Junction in |
| | SEQ ID NO: | SEQ ID NO: | SEQ ID NO: |
| Fusion transcript | X | X | (X + 1000) |
| ASCC1\|51008_MICU1\|10367 | seq_304 | 871-872 | 1178-1179 |
| ASCC1\|51008_MICU1\|10367 | seq_300 | 955-956 | 1223-1224 |
| ASCC1\|51008_MICU1\|10367 | seq_299 | 489-490 | 796-797 |
| ASCC1\|51008_MICU1\|10367 | seq_308 | 616-617 | 659-660 |
| ASCC1\|51008_MICU1\|10367 | seq_301 | 234-235 | 277-278 |
| ASCC1\|51008_MICU1\|10367 | seq_302 | 573-574 | 841-842 |
| ASCC1\|51008_MICU1\|10367 | seq_303 | 489-490 | 796-797 |
| ASCC1\|51008_MICU1\|10367 | seq_309 | 573-574 | 841-842 |
| ASCC1\|51008_MICU1\|10367 | seq_305 | 934-935 | 1218-1219 |
| ASCC1\|51008_MICU1\|10367 | seq_307 | 552-553 | 836-837 |
| ASCC1\|51008_MICU1\|10367 | seq_306 | 552-553 | 836-837 |
| ASCC1\|51008_MICU1\|10367 | seq_310 | 234-235 | 277-278 |
| CMTM7\|112616_CMTM8\|152189 | seq_350 | 333-334 | 569-570 |
| CMTM7\|112616_CMTM8\|152189 | seq_351 | 333-334 | 569-570 |
| CMTM7\|112616_CMTM8\|152189 | seq_349 | 333-334 | 569-570 |
| CMTM7\|112616_CMTM8\|152189 | seq_348 | 159-160 | 395-396 |
| MYH9\|4627_TXN2\|25828 | seq_521 | 333-334 | 564-565 |
| MYH9\|4627_TXN2\|25828 | seq_522 | 0-1 | 721-722 |
| PPFIBP1\|8496_C12orf70\|341346 | seq_810 | NA | 254-255 |
| FLJ22447\|400221_PRKCH\|5583 | seq_134 | 0-1 | 221-222 |
| FLJ22447\|400221_PRKCH\|5583 | seq_802 | NA | 221-222 |
| FLJ22447\|400221_PRKCH\|5583 | seq_133 | 0-1 | 221-222 |
| FLJ22447\|400221_PRKCH\|5583 | seq_803 | NA | 221-222 |
| KAT6B\|23522_ADK\|132 | seq_641 | 621-622 | 949-950 |
| KAT6B\|23522_ADK\|132 | seq_642 | 621-622 | 1114-1115 |
| USP22\|23326_MYH10\|4628 | seq_165 | 690-691 | 894-895 |
| USP22\|23326_MYH10\|4628 | seq_163 | 690-691 | 894-895 |
| USP22\|23326_MYH10\|4628 | seq_166 | 654-655 | 654-655 |
| USP22\|23326_MYH10\|4628 | seq_169 | 375-376 | 959-960 |
| USP22\|23326_MYH10\|4628 | seq_162 | 654-655 | 654-655 |
| USP22\|23326_MYH10\|4628 | seq_161 | 690-691 | 894-895 |
| USP22\|23326_MYH10\|4628 | seq_168 | 375-376 | 959-960 |
| USP22\|23326_MYH10\|4628 | seq_164 | 654-655 | 654-655 |
| USP22\|23326_MYH10\|4628 | seq_167 | 375-376 | 959-960 |
| TTYH3\|80727_MAD1L1\|8379 | seq_653 | 123-124 | 310-311 |
| TTYH3\|80727_MAD1L1\|8379 | seq_651 | 123-124 | 310-311 |
| TTYH3\|80727_MAD1L1\|8379 | seq_648 | 123-124 | 310-311 |
| TTYH3\|80727_MAD1L1\|8379 | seq_644 | 123-124 | 310-311 |
| TTYH3\|80727_MAD1L1\|8379 | seq_654 | 123-124 | 310-311 |
| TTYH3\|80727_MAD1L1\|8379 | seq_652 | 123-124 | 310-311 |
| TTYH3\|80727_MAD1L1\|8379 | seq_645 | 123-124 | 310-311 |
| TTYH3\|80727_MAD1L1\|8379 | seq_657 | 123-124 | 310-311 |
| TTYH3\|80727_MAD1L1\|8379 | seq_656 | 123-124 | 310-311 |
| TTYH3\|80727_MAD1L1\|8379 | seq_655 | 405-406 | 592-593 |
| TTYH3\|80727_MAD1L1\|8379 | seq_647 | 123-124 | 310-311 |
| TTYH3\|80727_MAD1L1\|8379 | seq_658 | 405-406 | 592-593 |
| TTYH3\|80727_MAD1L1\|8379 | seq_643 | 123-124 | 310-311 |
| TTYH3\|80727_MAD1L1\|8379 | seq_646 | 123-124 | 310-311 |
| TTYH3\|80727_MAD1L1\|8379 | seq_649 | 123-124 | 310-311 |
| TTYH3\|80727_MAD1L1\|8379 | seq_650 | 405-406 | 592-593 |
| NCOA3\|8202_EYA2\|2139 | seq_391 | 0-1 | 242-243 |
| NCOA3\|8202_EYA2\|2139 | seq_393 | 0-1 | 242-243 |
| NCOA3\|8202_EYA2\|2139 | seq_392 | 0-1 | 163-164 |
| EXOC4\|60412_CHCHD3\|54927 | seq_137 | 1514-1515 | 1549-1550 |
| EXOC4\|60412_CHCHD3\|54927 | seq_152 | 1182-1183 | 1217-1218 |
| EXOC4\|60412_CHCHD3\|54927 | seq_139 | 110-111 | 360-361 |
| EXOC4\|60412_CHCHD3\|54927 | seq_143 | 879-880 | 1225-1226 |
| EXOC4\|60412_CHCHD3\|54927 | seq_154 | 344-345 | 397-398 |
| EXOC4\|60412_CHCHD3\|54927 | seq_150 | 1182-1183 | 1217-1218 |
| EXOC4\|60412_CHCHD3\|54927 | seq_149 | 1182-1183 | 1217-1218 |
| EXOC4\|60412_CHCHD3\|54927 | seq_148 | 879-880 | 1225-1226 |
| EXOC4\|60412_CHCHD3\|54927 | seq_155 | 1182-1183 | 1217-1218 |
| EXOC4\|60412_CHCHD3\|54927 | seq_146 | 879-880 | 1225-1226 |
| EXOC4\|60412_CHCHD3\|54927 | seq_142 | 1211-1212 | 1557-1558 |
| EXOC4\|60412_CHCHD3\|54927 | seq_136 | 110-111 | 360-361 |
| EXOC4\|60412_CHCHD3\|54927 | seq_153 | 1182-1183 | 1217-1218 |
| EXOC4\|60412_CHCHD3\|54927 | seq_145 | 879-880 | 1225-1226 |
| EXOC4\|60412_CHCHD3\|54927 | seq_151 | 110-111 | 360-361 |
| EXOC4\|60412_CHCHD3\|54927 | seq_159 | 1211-1212 | 1557-1558 |
| EXOC4\|60412_CHCHD3\|54927 | seq_140 | 344-345 | 397-398 |
| EXOC4\|60412_CHCHD3\|54927 | seq_144 | 1514-1515 | 1549-1550 |
| EXOC4\|60412_CHCHD3\|54927 | seq_147 | 1211-1212 | 1557-1558 |
| EXOC4\|60412_CHCHD3\|54927 | seq_158 | 1514-1515 | 1549-1550 |
| EXOC4\|60412_CHCHD3\|54927 | seq_156 | 344-345 | 397-398 |
| WASF2\|10163_AHDC1\|27245 | seq_206 | 0-1 | 355-356 |
| WASF2\|10163_AHDC1\|27245 | seq_205 | 0-1 | 355-356 |
| MLL5\|55904_LHFPL3\|375612 | seq_637 | 411-412 | 411-412 |
| MLL5\|55904_LHFPL3\|375612 | seq_634 | 411-412 | 411-412 |
| MLL5\|55904_LHFPL3\|375612 | seq_635 | 1623-1624 | 2083-2084 |
| MLL5\|55904_LHFPL3\|375612 | seq_633 | 1185-1186 | 2246-2247 |
| MLL5\|55904_LHFPL3\|375612 | seq_636 | 1185-1186 | 2246-2247 |
| MLL5\|55904_LHFPL3\|375612 | seq_638 | 1623-1624 | 2083-2084 |
| PPP1CB\|5500_PLB1\|151056 | seq_194 | 100-101 | 205-206 |
| PPP1CB\|5500_PLB1\|151056 | seq_195 | 184-185 | 549-550 |
| PPP1CB\|5500_PLB1\|151056 | seq_202 | 52-53 | 417-418 |
| PPP1CB\|5500_PLB1\|151056 | seq_191 | 52-53 | 417-418 |
| PPP1CB\|5500_PLB1\|151056 | seq_196 | 52-53 | 417-418 |
| PPP1CB\|5500_PLB1\|151056 | seq_190 | 100-101 | 205-206 |
| PPP1CB\|5500_PLB1\|151056 | seq_192 | 52-53 | 417-418 |
| PPP1CB\|5500_PLB1\|151056 | seq_199 | 52-53 | 417-418 |
| PPP1CB\|5500_PLB1\|151056 | seq_200 | 100-101 | 205-206 |
| PPP1CB\|5500_PLB1\|151056 | seq_198 | 52-53 | 417-418 |
| PPP1CB\|5500_PLB1\|151056 | seq_197 | 52-53 | 417-418 |
| PPP1CB\|5500_PLB1\|151056 | seq_188 | 184-185 | 549-550 |
| PPP1CB\|5500_PLB1\|151056 | seq_201 | 184-185 | 549-550 |
| PPP1CB\|5500_PLB1\|151056 | seq_193 | 100-101 | 205-206 |
| PPP1CB\|5500_PLB1\|151056 | seq_189 | 184-185 | 549-550 |
| IFT43\|112752_TTLL5\|23093 | seq_292 | 147-148 | 181-182 |
| IFT43\|112752_TTLL5\|23093 | seq_293 | 147-148 | 181-182 |
| IFT43\|112752_TTLL5\|23093 | seq_291 | 215-216 | 249-250 |
| FAM190A\|401145_MMRN1\|22915 | seq_687 | 0-1 | 299-300 |
| QKI\|9444_PACRG\|135138 | seq_278 | 402-403 | 953-954 |
| QKI\|9444_PACRG\|135138 | seq_276 | 402-403 | 953-954 |
| QKI\|9444_PACRG\|135138 | seq_279 | 285-286 | 836-837 |
| QKI\|9444_PACRG\|135138 | seq_277 | 142-143 | 693-694 |
| FAM3B\|54097_BACE2\|25825 | seq_345 | 618-619 | 764-765 |
| FAM3B\|54097_BACE2\|25825 | seq_347 | 618-619 | 764-765 |
| FAM3B\|54097_BACE2\|25825 | seq_346 | 205-206 | 205-206 |
| FAM3B\|54097_BACE2\|25825 | seq_343 | 618-619 | 764-765 |
| FAM3B\|54097_BACE2\|25825 | seq_342 | 474-475 | 620-621 |
| FAM3B\|54097_BACE2\|25825 | seq_340 | 474-475 | 620-621 |
| FAM3B\|54097_BACE2\|25825 | seq_341 | 474-475 | 620-621 |
| FAM3B\|54097_BACE2\|25825 | seq_344 | 163-164 | 309-310 |
| THSD4\|79875_LRRC49\|54839 | seq_213 | 464-465 | 543-544 |
| THSD4\|79875_LRRC49\|54839 | seq_212 | 99-100 | 178-179 |
| THSD4\|79875_LRRC49\|54839 | seq_208 | 99-100 | 178-179 |
| THSD4\|79875_LRRC49\|54839 | seq_207 | 174-175 | 688-689 |
| THSD4\|79875_LRRC49\|54839 | seq_209 | 29-30 | 108-109 |
| THSD4\|79875_LRRC49\|54839 | seq_214 | 174-175 | 688-689 |
| THSD4\|79875_LRRC49\|54839 | seq_210 | 1152-1153 | 1231-1232 |
| THSD4\|79875_LRRC49\|54839 | seq_215 | 1152-1153 | 1231-1232 |
| THSD4\|79875_LRRC49\|54839 | seq_211 | 99-100 | 178-179 |
| EIF2C2\|27161_PTK2\|5747 | seq_506 | 22-23 | 63-64 |
| EIF2C2\|27161_PTK2\|5747 | seq_505 | 0-1 | 63-64 |
| EIF2C2\|27161_PTK2\|5747 | seq_507 | 22-23 | 63-64 |
| EIF2C2\|27161_PTK2\|5747 | seq_504 | 22-23 | 63-64 |
| EIF2C2\|27161_PTK2\|5747 | seq_503 | 22-23 | 63-64 |
| EIF2C2\|27161_PTK2\|5747 | seq_509 | 0-1 | 63-64 |
| EIF2C2\|27161_PTK2\|5747 | seq_502 | 22-23 | 63-64 |
| EIF2C2\|27161_PTK2\|5747 | seq_508 | 22-23 | 63-64 |
| SLPI\|6590_WFDC2\|10406 | seq_532 | 394-395 | 416-417 |
| SLPI\|6590_WFDC2\|10406 | seq_533 | 244-245 | 266-267 |
| BMPR1B\|658_PDLIM5\|10611 | seq_466 | 1076-1077 | 1350-1351 |
| BMPR1B\|658_PDLIM5\|10611 | seq_453 | 585-586 | 739-740 |
| BMPR1B\|658_PDLIM5\|10611 | seq_455 | 0-1 | 257-258 |
| BMPR1B\|658_PDLIM5\|10611 | seq_473 | 0-1 | 257-258 |
| BMPR1B\|658_PDLIM5\|10611 | seq_472 | 0-1 | 257-258 |
| BMPR1B\|658_PDLIM5\|10611 | seq_457 | 143-144 | 297-298 |
| BMPR1B\|658_PDLIM5\|10611 | seq_459 | 0-1 | 257-258 |
| BMPR1B\|658_PDLIM5\|10611 | seq_470 | 0-1 | 257-258 |
| BMPR1B\|658_PDLIM5\|10611 | seq_461 | 1076-1077 | 1350-1351 |
| BMPR1B\|658_PDLIM5\|10611 | seq_456 | 585-586 | 655-656 |
| BMPR1B\|658_PDLIM5\|10611 | seq_458 | 585-586 | 739-740 |
| BMPR1B\|658_PDLIM5\|10611 | seq_469 | 1076-1077 | 1230-1231 |
| BMPR1B\|658_PDLIM5\|10611 | seq_464 | 585-586 | 859-860 |
| BMPR1B\|658_PDLIM5\|10611 | seq_467 | 0-1 | 162-163 |
| BMPR1B\|658_PDLIM5\|10611 | seq_462 | 585-586 | 859-860 |
| BMPR1B\|658_PDLIM5\|10611 | seq_463 | 0-1 | 162-163 |
| BMPR1B\|658_PDLIM5\|10611 | seq_454 | 1076-1077 | 1146-1147 |
| BMPR1B\|658_PDLIM5\|10611 | seq_474 | 0-1 | 257-258 |
| BMPR1B\|658_PDLIM5\|10611 | seq_465 | 1076-1077 | 1146-1147 |
| BMPR1B\|658_PDLIM5\|10611 | seq_475 | 585-586 | 655-656 |
| BMPR1B\|658_PDLIM5\|10611 | seq_471 | 143-144 | 213-214 |
| NSD1\|64324_ZNF346\|23567 | seq_26 | 5509-5510 | 5647-5648 |
| NSD1\|64324_ZNF346\|23567 | seq_25 | 7-8 | 695-696 |
| NSD1\|64324_ZNF346\|23567 | seq_12 | 4765-4766 | 4903-4904 |
| NSD1\|64324_ZNF346\|23567 | seq_41 | 1063-1064 | 1156-1157 |
| NSD1\|64324_ZNF346\|23567 | seq_24 | 4453-4454 | 5141-5142 |
| NSD1\|64324_ZNF346\|23567 | seq_33 | 2740-2741 | 3428-3429 |
| NSD1\|64324_ZNF346\|23567 | seq_28 | 3958-3959 | 4118-4119 |
| NSD1\|64324_ZNF346\|23567 | seq_35 | 256-257 | 416-417 |
| NSD1\|64324_ZNF346\|23567 | seq_20 | 256-257 | 416-417 |
| NSD1\|64324_ZNF346\|23567 | seq_32 | 1063-1064 | 1201-1202 |
| NSD1\|64324_ZNF346\|23567 | seq_30 | 3487-3488 | 3504-3505 |
| NSD1\|64324_ZNF346\|23567 | seq_29 | 4702-4703 | 4862-4863 |
| NSD1\|64324_ZNF346\|23567 | seq_31 | 7-8 | 695-696 |
| NSD1\|64324_ZNF346\|23567 | seq_37 | 5200-5201 | 5217-5218 |
| NSD1\|64324_ZNF346\|23567 | seq_17 | 2989-2990 | 3149-3150 |
| NSD1\|64324_ZNF346\|23567 | seq_18 | 3709-3710 | 4397-4398 |
| NSD1\|64324_ZNF346\|23567 | seq_14 | 3487-3488 | 3504-3505 |
| NSD1\|64324_ZNF346\|23567 | seq_10 | 4456-4457 | 4473-4474 |
| NSD1\|64324_ZNF346\|23567 | seq_7 | 7-8 | 695-696 |
| NSD1\|64324_ZNF346\|23567 | seq_13 | 2740-2741 | 3428-3429 |
| NSD1\|64324_ZNF346\|23567 | seq_15 | 3796-3797 | 3934-3935 |
| NSD1\|64324_ZNF346\|23567 | seq_11 | 4456-4457 | 4473-4474 |
| NSD1\|64324_ZNF346\|23567 | seq_23 | 3796-3797 | 3934-3935 |
| NSD1\|64324_ZNF346\|23567 | seq_16 | 256-257 | 416-417 |
| NSD1\|64324_ZNF346\|23567 | seq_21 | 3709-3710 | 4397-4398 |
| NSD1\|64324_ZNF346\|23567 | seq_6 | 4702-4703 | 4862-4863 |
| NSD1\|64324_ZNF346\|23567 | seq_19 | 2989-2990 | 3149-3150 |
| NSD1\|64324_ZNF346\|23567 | seq_34 | 4453-4454 | 5141-5142 |
| NSD1\|64324_ZNF346\|23567 | seq_38 | 4765-4766 | 4903-4904 |
| NSD1\|64324_ZNF346\|23567 | seq_8 | 1063-1064 | 1201-1202 |
| NSD1\|64324_ZNF346\|23567 | seq_27 | 5509-5510 | 5647-5648 |
| NSD1\|64324_ZNF346\|23567 | seq_39 | 5200-5201 | 5217-5218 |
| NSD1\|64324_ZNF346\|23567 | seq_22 | 3958-3959 | 4118-4119 |
| LMO7\|4008_UCHL3\|7347 | seq_666 | 69-70 | 404-405 |
| LMO7\|4008_UCHL3\|7347 | seq_668 | 345-346 | 364-365 |
| LMO7\|4008_UCHL3\|7347 | seq_665 | 366-367 | 1626-1627 |
| LMO7\|4008_UCHL3\|7347 | seq_663 | 210-211 | 545-546 |
| LMO7\|4008_UCHL3\|7347 | seq_669 | 618-619 | 1878-1879 |
| LMO7\|4008_UCHL3\|7347 | seq_670 | 69-70 | 404-405 |
| LMO7\|4008_UCHL3\|7347 | seq_667 | 225-226 | 1485-1486 |
| LMO7\|4008_UCHL3\|7347 | seq_664 | 462-463 | 797-798 |
| TNRC18\|84629_RNF216\|54476 | seq_811 | NA | 106-107 |
| TNRC18\|84629_RNF216\|54476 | seq_575 | 4833-4834 | 5182-5183 |
| LRBA\|987_SH3D19\|152503 | seq_535 | 216-217 | 501-502 |
| LRBA\|987_SH3D19\|152503 | seq_536 | 216-217 | 460-461 |
| LRBA\|987_SH3D19\|152503 | seq_534 | 216-217 | 501-502 |
| LRBA\|987_SH3D19\|152503 | seq_537 | 216-217 | 501-502 |
| NCOR2\|9612_SCARB1\|949 | seq_228 | 1479-1480 | 1800-1801 |
| NCOR2\|9612_SCARB1\|949 | seq_216 | 1482-1483 | 1754-1755 |
| NCOR2\|9612_SCARB1\|949 | seq_218 | 815-816 | 1136-1137 |
| NCOR2\|9612_SCARB1\|949 | seq_231 | 705-706 | 1026-1027 |
| NCOR2\|9612_SCARB1\|949 | seq_229 | 815-816 | 1087-1088 |
| NCOR2\|9612_SCARB1\|949 | seq_232 | 1479-1480 | 1800-1801 |
| NCOR2\|9612_SCARB1\|949 | seq_217 | 762-763 | 1034-1035 |
| NCOR2\|9612_SCARB1\|949 | seq_225 | 1479-1480 | 1800-1801 |
| NCOR2\|9612_SCARB1\|949 | seq_230 | 1479-1480 | 1800-1801 |
| NCOR2\|9612_SCARB1\|949 | seq_223 | 762-763 | 1083-1084 |
| NCOR2\|9612_SCARB1\|949 | seq_242 | 705-706 | 1026-1027 |
| NCOR2\|9612_SCARB1\|949 | seq_219 | 705-706 | 977-978 |
| NCOR2\|9612_SCARB1\|949 | seq_222 | 762-763 | 1083-1084 |
| NCOR2\|9612_SCARB1\|949 | seq_236 | 1482-1483 | 1599-1600 |
| NCOR2\|9612_SCARB1\|949 | seq_233 | 762-763 | 1083-1084 |
| NCOR2\|9612_SCARB1\|949 | seq_227 | 705-706 | 1026-1027 |
| NCOR2\|9612_SCARB1\|949 | seq_234 | 1876-1877 | 1993-1994 |
| NCOR2\|9612_SCARB1\|949 | seq_238 | 1873-1874 | 2194-2195 |
| NCOR2\|9612_SCARB1\|949 | seq_226 | 705-706 | 1026-1027 |
| NCOR2\|9612_SCARB1\|949 | seq_220 | 1479-1480 | 1800-1801 |
| NCOR2\|9612_SCARB1\|949 | seq_240 | 815-816 | 1136-1137 |
| NCOR2\|9612_SCARB1\|949 | seq_243 | 815-816 | 1136-1137 |
| NCOR2\|9612_SCARB1\|949 | seq_239 | 1482-1483 | 1599-1600 |
| NCOR2\|9612_SCARB1\|949 | seq_237 | 411-412 | 732-733 |
| NCOR2\|9612_SCARB1\|949 | seq_221 | 762-763 | 1083-1084 |
| NCOR2\|9612_SCARB1\|949 | seq_235 | 1482-1483 | 1803-1804 |
| NCOR2\|9612_SCARB1\|949 | seq_224 | 815-816 | 1136-1137 |
| EXT1\|2131_SAMD12\|401474 | seq_801 | NA | 1735-1736 |
| EXT1\|2131_SAMD12\|401474 | seq_800 | NA | 1735-1736 |
| MATR3\|9782_CTNNA1\|1495 | seq_105 | 0-1 | 162-163 |
| MATR3\|9782_CTNNA1\|1495 | seq_106 | 0-1 | 279-280 |
| SORL1\|6653_TECTA\|7007 | seq_5 | 1211-1212 | 1340-1341 |
| SORL1\|6653_TECTA\|7007 | seq_4 | 528-529 | 657-658 |
| SORL1\|6653_TECTA\|7007 | seq_3 | 528-529 | 657-658 |
| SORL1\|6653_TECTA\|7007 | seq_2 | 1685-1686 | 1814-1815 |
| SORL1\|6653_TECTA\|7007 | seq_1 | 758-759 | 887-888 |
| EIF3B\|8662_MAD1L1\|8379 | seq_121 | 2154-2155 | 2237-2238 |
| EIF3B\|8662_MAD1L1\|8379 | seq_130 | 1338-1339 | 1655-1656 |
| EIF3B\|8662_MAD1L1\|8379 | seq_123 | 2154-2155 | 2237-2238 |
| EIF3B\|8662_MAD1L1\|8379 | seq_128 | 1338-1339 | 1655-1656 |
| EIF3B\|8662_MAD1L1\|8379 | seq_132 | 2154-2155 | 2237-2238 |
| EIF3B\|8662_MAD1L1\|8379 | seq_116 | 1338-1339 | 1655-1656 |
| EIF3B\|8662_MAD1L1\|8379 | seq_124 | 2154-2155 | 2237-2238 |
| EIF3B\|8662_MAD1L1\|8379 | seq_122 | 2154-2155 | 2237-2238 |
| EIF3B\|8662_MAD1L1\|8379 | seq_131 | 1338-1339 | 1655-1656 |
| EIF3B\|8662_MAD1L1\|8379 | seq_125 | 0-1 | 1101-1102 |
| EIF3B\|8662_MAD1L1\|8379 | seq_119 | 1338-1339 | 1655-1656 |
| EIF3B\|8662_MAD1L1\|8379 | seq_126 | 1338-1339 | 1655-1656 |
| EIF3B\|8662_MAD1L1\|8379 | seq_117 | 1338-1339 | 1655-1656 |
| EIF3B\|8662_MAD1L1\|8379 | seq_127 | 2154-2155 | 2237-2238 |
| EIF3B\|8662_MAD1L1\|8379 | seq_129 | 2154-2155 | 2237-2238 |
| CD44\|960_PDHX\|8050 | seq_701 | 233-234 | 667-668 |
| CD44\|960_PDHX\|8050 | seq_700 | 261-262 | 695-696 |
| CD44\|960_PDHX\|8050 | seq_697 | 436-437 | 870-871 |
| CD44\|960_PDHX\|8050 | seq_699 | 436-437 | 870-871 |
| CD44\|960_PDHX\|8050 | seq_702 | 667-668 | 1101-1102 |
| CD44\|960_PDHX\|8050 | seq_705 | 67-68 | 501-502 |
| CD44\|960_PDHX\|8050 | seq_703 | 667-668 | 1101-1102 |
| CD44\|960_PDHX\|8050 | seq_704 | 67-68 | 501-502 |
| CD44\|960_PDHX\|8050 | seq_698 | 67-68 | 501-502 |
| C7orf50\|84310_MAD1L1\|8379 | seq_354 | 129-130 | 199-200 |
| C7orf50\|84310_MAD1L1\|8379 | seq_352 | 129-130 | 170-171 |
| C7orf50\|84310_MAD1L1\|8379 | seq_355 | 129-130 | 199-200 |
| C7orf50\|84310_MAD1L1\|8379 | seq_353 | 129-130 | 189-190 |
| CAPZA2\|830_MET\|4233 | seq_672 | 39-40 | 142-143 |
| CAPZA2\|830_MET\|4233 | seq_678 | 39-40 | 142-143 |
| CAPZA2\|830_MET\|4233 | seq_673 | 103-104 | 206-207 |
| CAPZA2\|830_MET\|4233 | seq_681 | 0-1 | 142-143 |
| CAPZA2\|830_MET\|4233 | seq_674 | 39-40 | 142-143 |
| CAPZA2\|830_MET\|4233 | seq_675 | 39-40 | 142-143 |
| CAPZA2\|830_MET\|4233 | seq_684 | 39-40 | 142-143 |
| CAPZA2\|830_MET\|4233 | seq_676 | 39-40 | 142-143 |
| CAPZA2\|830_MET\|4233 | seq_683 | 39-40 | 142-143 |
| CAPZA2\|830_MET\|4233 | seq_680 | 39-40 | 142-143 |
| CAPZA2\|830_MET\|4233 | seq_682 | 39-40 | 142-143 |
| CAPZA2\|830_MET\|4233 | seq_677 | 39-40 | 142-143 |
| CAPZA2\|830_MET\|4233 | seq_671 | 39-40 | 142-143 |
| CAPZA2\|830_MET\|4233 | seq_679 | 585-586 | 688-689 |
| FRS2\|10818_LYZ\|4069 | seq_806 | NA | 182-183 |
| FRS2\|10818_LYZ\|4069 | seq_807 | NA | 278-279 |
| KIF26B\|55083_SMYD3\|64754 | seq_260 | 204-205 | 311-312 |
| KIF26B\|55083_SMYD3\|64754 | seq_249 | 1350-1351 | 1790-1791 |
| KIF26B\|55083_SMYD3\|64754 | seq_245 | 4677-4678 | 4677-4678 |
| KIF26B\|55083_SMYD3\|64754 | seq_252 | 399-400 | 773-774 |
| KIF26B\|55083_SMYD3\|64754 | seq_259 | 204-205 | 311-312 |
| KIF26B\|55083_SMYD3\|64754 | seq_255 | 1350-1351 | 1790-1791 |
| KIF26B\|55083_SMYD3\|64754 | seq_256 | 999-1000 | 1439-1440 |
| KIF26B\|55083_SMYD3\|64754 | seq_254 | 3549-3550 | 3549-3550 |
| KIF26B\|55083_SMYD3\|64754 | seq_248 | 465-466 | 905-906 |
| KIF26B\|55083_SMYD3\|64754 | seq_251 | 1166-1167 | 1606-1607 |
| KIF26B\|55083_SMYD3\|64754 | seq_253 | 1350-1351 | 1790-1791 |
| KIF26B\|55083_SMYD3\|64754 | seq_258 | 204-205 | 311-312 |
| KIF26B\|55083_SMYD3\|64754 | seq_247 | 465-466 | 905-906 |
| KIF26B\|55083_SMYD3\|64754 | seq_246 | 465-466 | 905-906 |
| KIF26B\|55083_SMYD3\|64754 | seq_250 | 465-466 | 905-906 |
| LYPD6\|130574_LYPD6B\|130576 | seq_61 | 0-1 | 506-507 |
| LYPD6\|130574_LYPD6B\|130576 | seq_62 | 0-1 | 610-611 |
| ZBTB20\|26137_LSAMP\|4045 | seq_812 | NA | 62-63 |
| SRPK2\|6733_PUS7\|54517 | seq_184 | 71-72 | 159-160 |
| SRPK2\|6733_PUS7\|54517 | seq_183 | 71-72 | 159-160 |
| ARL15\|54622_NDUFS4\|4724 | seq_798 | 193-194 | 287-288 |
| ARL15\|54622_NDUFS4\|4724 | seq_796 | 253-254 | 347-348 |
| ARL15\|54622_NDUFS4\|4724 | seq_797 | 48-49 | 142-143 |
| ARL15\|54622_NDUFS4\|4724 | seq_799 | 462-463 | 556-557 |
| LOC100499467\|100499467_SLC39A11\|201266 | seq_808 | NA | 602-603 |
| LOC100499467\|100499467_SLC39A11\|201266 | seq_809 | NA | 602-603 |
| FRMD6\|122786_LOC283553\|283553 | seq_805 | NA | 347-348 |
| FRMD6\|122786_LOC283553\|283553 | seq_804 | NA | 284-285 |
| SH3PXD2A\|9644_OBFC1\|79991 | seq_101 | 72-73 | 212-213 |
| SH3PXD2A\|9644_OBFC1\|79991 | seq_102 | 306-307 | 446-447 |
| SH3PXD2A\|9644_OBFC1\|79991 | seq_100 | 96-97 | 163-164 |
| COL14A1\|7373_DEPTOR\|64798 | seq_275 | 2349-2350 | 2614-2615 |
| COL14A1\|7373_DEPTOR\|64798 | seq_268 | 1737-1738 | 2002-2003 |
| COL14A1\|7373_DEPTOR\|64798 | seq_270 | 88-89 | 353-354 |
| COL14A1\|7373_DEPTOR\|64798 | seq_272 | 436-437 | 701-702 |
| COL14A1\|7373_DEPTOR\|64798 | seq_269 | 205-206 | 470-471 |
| COL14A1\|7373_DEPTOR\|64798 | seq_267 | 1513-1514 | 2043-2044 |
| COL14A1\|7373_DEPTOR\|64798 | seq_273 | 771-772 | 1016-1017 |
| COL14A1\|7373_DEPTOR\|64798 | seq_274 | 1383-1384 | 1913-1914 |
| COL14A1\|7373_DEPTOR\|64798 | seq_271 | 877-878 | 1142-1143 |
| COL14A1\|7373_DEPTOR\|64798 | seq_266 | 2479-2480 | 2744-2745 |
| ASH1L\|55870_GON4L\|54856 | seq_49 | 420-421 | 900-901 |
| ASH1L\|55870_GON4L\|54856 | seq_45 | 420-421 | 900-901 |
| ASH1L\|55870_GON4L\|54856 | seq_54 | 420-421 | 900-901 |
| ASH1L\|55870_GON4L\|54856 | seq_51 | 420-421 | 678-679 |
| ASH1L\|55870_GON4L\|54856 | seq_46 | 420-421 | 678-679 |
| ASH1L\|55870_GON4L\|54856 | seq_44 | 420-421 | 900-901 |
| ASH1L\|55870_GON4L\|54856 | seq_50 | 420-421 | 900-901 |
| ASH1L\|55870_GON4L\|54856 | seq_53 | 420-421 | 900-901 |
| ASH1L\|55870_GON4L\|54856 | seq_48 | 420-421 | 900-901 |
| ASH1L\|55870_GON4L\|54856 | seq_60 | 420-421 | 900-901 |
| ASH1L\|55870_GON4L\|54856 | seq_58 | 420-421 | 678-679 |
| ASH1L\|55870_GON4L\|54856 | seq_55 | 420-421 | 900-901 |
| ZC3H7A\|29066_BCAR4\|400500 | seq_319 | 0-1 | 135-136 |
| STX5\|6811_WDR74\|54663 | seq_525 | 423-424 | 580-581 |
| STX5\|6811_WDR74\|54663 | seq_529 | 0-1 | 138-139 |
| STX5\|6811_WDR74\|54663 | seq_527 | 135-136 | 336-337 |
| STX5\|6811_WDR74\|54663 | seq_526 | 0-1 | 592-593 |
| STX5\|6811_WDR74\|54663 | seq_531 | 0-1 | 1065-1066 |
| STX5\|6811_WDR74\|54663 | seq_530 | 423-424 | 580-581 |
| STX5\|6811_WDR74\|54663 | seq_528 | 135-136 | 336-337 |
| TANC1\|85461_PKP4\|8502 | seq_358 | 0-1 | 79-80 |
| TANC1\|85461_PKP4\|8502 | seq_356 | 0-1 | 79-80 |
| TANC1\|85461_PKP4\|8502 | seq_363 | 0-1 | 79-80 |
| TANC1\|85461_PKP4\|8502 | seq_359 | 0-1 | 79-80 |
| TANC1\|85461_PKP4\|8502 | seq_364 | 0-1 | 79-80 |
| TANC1\|85461_PKP4\|8502 | seq_366 | 0-1 | 79-80 |
| TANC1\|85461_PKP4\|8502 | seq_367 | 0-1 | 79-80 |
| PDE4D\|5144_DEPDC1B\|55789 | seq_296 | 78-79 | 489-490 |
| PDE4D\|5144_DEPDC1B\|55789 | seq_294 | 42-43 | 288-289 |
| PDE4D\|5144_DEPDC1B\|55789 | seq_295 | 42-43 | 288-289 |
| PDE4D\|5144_DEPDC1B\|55789 | seq_298 | 0-1 | 293-294 |
| PDE4D\|5144_DEPDC1B\|55789 | seq_297 | 78-79 | 489-490 |
| TFDP1\|7027_TMCO3\|55002 | seq_286 | 186-187 | 405-406 |
| TFDP1\|7027_TMCO3\|55002 | seq_289 | 23-24 | 293-294 |
| TFDP1\|7027_TMCO3\|55002 | seq_288 | 0-1 | 119-120 |
| TFDP1\|7027_TMCO3\|55002 | seq_282 | 0-1 | 119-120 |
| TFDP1\|7027_TMCO3\|55002 | seq_290 | 79-80 | 298-299 |
| TFDP1\|7027_TMCO3\|55002 | seq_284 | 186-187 | 405-406 |
| TFDP1\|7027_TMCO3\|55002 | seq_287 | 186-187 | 405-406 |
| TFDP1\|7027_TMCO3\|55002 | seq_285 | 79-80 | 298-299 |
| TFDP1\|7027_TMCO3\|55002 | seq_283 | 79-80 | 298-299 |
| TFDP1\|7027_TMCO3\|55002 | seq_280 | 186-187 | 405-406 |
| TFDP1\|7027_TMCO3\|55002 | seq_281 | 12-13 | 231-232 |
| SMARCC1\|6599_MAP4\|4134 | seq_73 | 1993-1994 | 2210-2211 |
| SMARCC1\|6599_MAP4\|4134 | seq_82 | 1993-1994 | 2210-2211 |
| SMARCC1\|6599_MAP4\|4134 | seq_76 | 315-316 | 433-434 |
| SMARCC1\|6599_MAP4\|4134 | seq_84 | 2320-2321 | 2438-2439 |
| SMARCC1\|6599_MAP4\|4134 | seq_74 | 1993-1994 | 2210-2211 |
| SMARCC1\|6599_MAP4\|4134 | seq_99 | 315-316 | 433-434 |
| SMARCC1\|6599_MAP4\|4134 | seq_65 | 1993-1994 | 2210-2211 |
| SMARCC1\|6599_MAP4\|4134 | seq_83 | 195-196 | 313-314 |
| SMARCC1\|6599_MAP4\|4134 | seq_88 | 1993-1994 | 2210-2211 |
| SMARCC1\|6599_MAP4\|4134 | seq_70 | 195-196 | 313-314 |
| SMARCC1\|6599_MAP4\|4134 | seq_81 | 1993-1994 | 2210-2211 |
| SMARCC1\|6599_MAP4\|4134 | seq_89 | 2320-2321 | 2438-2439 |
| SMARCC1\|6599_MAP4\|4134 | seq_67 | 1993-1994 | 2210-2211 |
| SMARCC1\|6599_MAP4\|4134 | seq_96 | 1993-1994 | 2210-2211 |
| SMARCC1\|6599_MAP4\|4134 | seq_90 | 1993-1994 | 2210-2211 |
| SMARCC1\|6599_MAP4\|4134 | seq_64 | 2320-2321 | 2438-2439 |
| SMARCC1\|6599_MAP4\|4134 | seq_87 | 1993-1994 | 2210-2211 |
| SMARCC1\|6599_MAP4\|4134 | seq_66 | 2320-2321 | 2438-2439 |
| SMARCC1\|6599_MAP4\|4134 | seq_97 | 2320-2321 | 2438-2439 |
| SMARCC1\|6599_MAP4\|4134 | seq_95 | 2320-2321 | 2438-2439 |
| SMARCC1\|6599_MAP4\|4134 | seq_71 | 1993-1994 | 2210-2211 |
| SMARCC1\|6599_MAP4\|4134 | seq_79 | 2320-2321 | 2438-2439 |
| SMARCC1\|6599_MAP4\|4134 | seq_85 | 2320-2321 | 2438-2439 |
| SMARCC1\|6599_MAP4\|4134 | seq_68 | 195-196 | 313-314 |
| SMARCC1\|6599_MAP4\|4134 | seq_69 | 1993-1994 | 2210-2211 |
| SMARCC1\|6599_MAP4\|4134 | seq_77 | 2320-2321 | 2438-2439 |
| SMARCC1\|6599_MAP4\|4134 | seq_98 | 315-316 | 433-434 |
| SMARCC1\|6599_MAP4\|4134 | seq_86 | 2320-2321 | 2438-2439 |
| SMARCC1\|6599_MAP4\|4134 | seq_75 | 2320-2321 | 2438-2439 |
| SMARCC1\|6599_MAP4\|4134 | seq_91 | 2320-2321 | 2438-2439 |
| SMARCC1\|6599_MAP4\|4134 | seq_78 | 2320-2321 | 2438-2439 |
| SMARCC1\|6599_MAP4\|4134 | seq_80 | 1993-1994 | 2210-2211 |
| SMARCC1\|6599_MAP4\|4134 | seq_72 | 2320-2321 | 2438-2439 |
| SMARCC1\|6599_MAP4\|4134 | seq_94 | 1993-1994 | 2210-2211 |
| SMARCC1\|6599_MAP4\|4134 | seq_93 | 1993-1994 | 2210-2211 |
| SMARCC1\|6599_MAP4\|4134 | seq_92 | 2320-2321 | 2438-2439 |
| HP1BP3\|50809_EIF4G3\|8672 | seq_715 | 0-1 | 212-213 |
| HP1BP3\|50809_EIF4G3\|8672 | seq_718 | 54-55 | 1504-1505 |
| HP1BP3\|50809_EIF4G3\|8672 | seq_719 | 0-1 | 732-733 |
| HP1BP3\|50809_EIF4G3\|8672 | seq_717 | 0-1 | 446-447 |
| HP1BP3\|50809_EIF4G3\|8672 | seq_716 | 0-1 | 112-113 |
| DNAJC24\|120526_IMMP1L\|196294 | seq_813 | 108-109 | 227-228 |
| GRB7\|2886_ERBB2\|2064 | seq_814 | 1452-1453 | 1727-1728 |
| GRB7\|2886_ERBB2\|2064 | seq_815 | 0-1 | 70-71 |
| GRB7\|2886_ERBB2\|2064 | seq_816 | 809-810 | 1727-1728 |
| GRB7\|2886_ERBB2\|2064 | seq_817 | 155-156 | 430-431 |
| GRB7\|2886_ERBB2\|2064 | seq_818 | 0-1 | 70-71 |
| GRB7\|2886_ERBB2\|2064 | seq_819 | 155-156 | 430-431 |
| GRB7\|2886_ERBB2\|2064 | seq_820 | 0-1 | 225-226 |
| GRB7\|2886_ERBB2\|2064 | seq_821 | 0-1 | 225-226 |
| GRB7\|2886_ERBB2\|2064 | seq_822 | 0-1 | 70-71 |
| GRB7\|2886_ERBB2\|2064 | seq_823 | 0-1 | 225-226 |
| GRB7\|2886_ERBB2\|2064 | seq_824 | 0-1 | 225-226 |
| LITAF\|9516_BCAR4\|400500 | seq_825 | 0-1 | 65-66 |
| LITAF\|9516_BCAR4\|400500 | seq_826 | 0-1 | 65-66 |
| LITAF\|9516_BCAR4\|400500 | seq_827 | 0-1 | 129-130 |
| LITAF\|9516_BCAR4\|400500 | seq_828 | 0-1 | 228-229 |
| LYPD6\|130574_LYPD6B\|130576 | seq_829 | 0-1 | 208-209 |
| LYPD6\|130574_LYPD6B\|130576 | seq_830 | 0-1 | 208-209 |
| LYPD6\|130574_LYPD6B\|130576 | seq_831 | 0-1 | 208-209 |
| LYPD6\|130574_LYPD6B\|130576 | seq_832 | 0-1 | 709-710 |
| LYPD6\|130574_LYPD6B\|130576 | seq_833 | 0-1 | 218-219 |
| LYPD6\|130574_LYPD6B\|130576 | seq_834 | 0-1 | 610-611 |
| LYPD6\|130574_LYPD6B\|130576 | seq_835 | 0-1 | 709-710 |
| REXO1\|57455_KLF16\|83855 | seq_836 | 157-158 | 252-253 |
| RGNEF\|64283_BTF3\|689 | seq_837 | 475-476 | 651-652 |
| RGNEF\|64283_BTF3\|689 | seq_838 | 33-34 | 209-210 |
| RGNEF\|64283_BTF3\|689 | seq_839 | 0-1 | 165-166 |
| RGNEF\|64283_BTF3\|689 | seq_840 | 33-34 | 209-210 |
| SLPI\|6590_WFDC2\|10406 | seq_841 | 244-245 | 266-267 |
| SLPI\|6590_WFDC2\|10406 | seq_842 | 394-395 | 416-417 |
| TYMS\|7298_SEPT9\|10801 | seq_843 | 454-455 | 593-594 |
| WASF2\|10163_IFI6\|2537 | seq_844 | 0-1 | 182-183 |

“0-1” or “NA” indicates no junction found in the indicated sequence
SEQ ID NO: X is the SEQ ID NO: of the sequence listing. For example, “seq_304” refers to SEQ ID NO: 304 of the sequence listing. SEQ ID NO: (X + 1000) is the SEQ ID NO: of the sequence listing with 1000 added to the X in the same row. For example, wherein SEQ ID NO: X is “seq_304” SEQ ID NO: (X + 1000) refers to SEQ ID NO: 1304 of the sequence listing.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted.

Recitation of ranges of values herein are merely intended to serve as a shorthand method of referring individually to each separate value falling within the range and each endpoint, unless otherwise indicated herein, and each separate value and endpoint is incorporated into the specification as if it were individually recited herein.

All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. An fusion transcript encoded by a nucleic acid molecule comprising a general structure A-B, wherein structure A is a portion of a gene listed in Column A of Table 1 and structure B is a portion of a gene listed in Column B of Table 1, wherein the gene listed in Column A and the gene listed in Column B are listed in the same row of Table 1, wherein structure B is located immediately 3′ to structure A.

2. The fusion transcript of claim 1, comprising a nucleotide sequence which is the reverse complement RNA of any one of SEQ ID NOs: 1 to 799 or the reverse complement of any one of SEQ ID NOs: 1001 to 1799.

3. The fusion transcript of claim 2, comprising a nucleotide sequence of any one of SEQ ID NOs: 2001 to 2799.

4. The fusion transcript of claim 1, comprising a nucleotide sequence which is the reverse complement RNA of any one of SEQ ID NOs: 800-844 or the reverse complement of any one of SEQ ID NOs: 1800 to 1844.

5. The fusion transcript of claim 4, comprising a nucleotide sequence of any one of SEQ ID NOs: 2800-2844.

6. The fusion transcript of claim 1, wherein the gene listed in Column A and the gene listed in Column B are listed in the same row of Table 1 and the row is marked with an asterisk in the 2^{nd} column from the left of Table 1.

7. The fusion transcript of claim 1, wherein the gene listed in Column A and the gene listed in Column B are listed in the same row of Table 1 and the row is not marked with “#” in the 3^{rd} column from the left of Table 1.

8. The fusion transcript of claim 1, wherein the gene listed in Column A and the gene listed in Column B are listed in the same row of Table 1 and the row is not marked with “̂” in the 4^{th} column from the left of Table 1.

9. The fusion transcript of claim 1, wherein structure A is a portion of a gene listed in Column A of Table 2 and structure B is a portion of a gene listed in Column B of Table 2, wherein the gene listed in Column A and the gene listed in Column B are listed in the same row of Table 2, wherein structure B is located immediately 3′ to structure A.

10. The fusion transcript of claim 1, wherein structure A is a portion of a gene listed in Column A of Table 3 and structure B is a portion of a gene listed in Column B of Table 3, wherein the gene listed in Column A and the gene listed in Column B are listed in the same row of Table 3, wherein structure B is located immediately 3′ to structure A.

11. The fusion transcript of claim 1, wherein structure A is a portion of a gene listed in Column A of Table 4 and structure B is a portion of a gene listed in Column B of Table 4, wherein the gene listed in Column A and the gene listed in Column B are listed in the same row of Table 4, wherein structure B is located immediately 3′ to structure A.

12. The fusion transcript of claim 1, having a junction as described in Table 5.

13.-23. (canceled)

24. A binding agent that specifically binds to DI a fusion transcript of claim 1 or (ii) a nucleic acid encoding the fusion transcript or (iii) a polypeptide encoded by the fusion transcript.

25. The binding agent of claim 24, which binds to a junction of the fusion transcript or the cDNA thereof.

26. A kit comprising a binding agent of claim 24.

27.-36. (canceled)

37. The method of claim 39, comprising
(i) contacting a binding agent that binds to a fusion transcript or a nucleic acid molecule encoding the fusion transcript with a sample obtained from the subject, wherein the binding agent specifically binds to a fusion transcript, and
(ii) determining
(a) the structure of the molecule bound to the binding agent or
(b) the presence or absence of a double stranded nucleic acid molecule comprising the binding agent and the fusion transcript, when the binding agent binds to a junction the fusion transcript,
wherein a cancer or tumor is detected in the subject, when the structure of the molecule is the structure of the fusion transcript or when the double stranded nucleic acid molecule is determined as present.

38. The method of claim 39, comprising
(i) generating a population of cDNAs from total cellular RNA isolated from cells of a sample obtained from the subject,
(ii) combining a binding agent that binds to a fusion transcript or a nucleic acid molecule encoding the fusion transcript, with the population of cDNAs, and
(iii) determining the structure of the nucleic acid bound to the binding agent or, when the binding agent specifically binds to a sequence comprising a junction of the nucleic acid encoding the fusion transcript, determining the presence or absence of a double stranded nucleic acid molecule comprising the binding agent and the nucleic acid,
wherein a cancer or tumor is detected in the subject, when the structure of the nucleic acid bound to the binding agent is the structure of the nucleic acid of any one of claims 14 to 16, or when the double stranded nucleic acid molecule is determined as present.

39. A method of detecting a cancer or a tumor in a subject, comprising assaying a sample obtained from the subject for expression of a fusion transcript of claim 1, expression of a polypeptide of encoded by the fusion transcript, or presence of a nucleic acid molecule of encoding the fusion transcript, wherein a cancer or tumor is detected when the sample is determined as positive for expression of the fusion transcript or polypeptide or for presence of the nucleic acid molecule.

40. The method of claim 39, further comprising administering to the subject an anti-cancer therapeutic agent in an amount effective for treating a cancer or tumor, when the sample is determined as positive for expression of the fusion transcript or fusion polypeptide or for presence of the nucleic acid molecule and/or determining a subject's need for an anti-cancer therapeutic agent, wherein the subject is determined as needing an anti-cancer therapeutic agent, when the sample is determined as positive for expression of the fusion transcript or fusion polypeptide or for presence of the nucleic acid molecule.

41. (canceled)

42. (canceled)

43. The method of claim 39, wherein the tumor is a tumor from adrenocortical carcinoma, bladder urothelial carcinoma, breast invasive carcinoma, cervical squamous cell carcinoma, colon adenocarcinoma, lymphoid neoplasm diffuse large B-cell, glioblastoma multiforme, head and neck squamous cell carcinoma, kidney chromophobe, kidney renal clear cell carcinoma, kidney renal papillary cell carcinoma, acute myeloid leukemia, brain lower grade glioma, liver hepatocellular carcinoma, lung adenocarcinoma, lung squamous cell carcinoma, ovarian serous cystadenocarcinoma, pancreatic adenocarcinoma, prostate adenocarcinoma, rectum adenocarcinoma, sarcoma, skin cutaneous melanoma, stomach adenocarcinoma, thyroid carcinoma, uterine corpus endometrial carcinoma, or uterine carcinosarcoma.
